(19) United States
(12) Patent Application Publication
(10) Pub. No.: US 2017/0211081 A1
(43) Pub. Date: Jul. 27, 2017

(54) FOXTAIL MOSAIC VIRUS-BASED VECTORS FOR GENE SILENCING AND GENE EXPRESSION

(71) Applicant: Iowa State University Research Foundation, Inc., Ames, IA (US)

(72) Inventors: John Hemmingson Hill, Ames, IA (US); Chunquan Zhang, Ames, IA (US); Steven Alan Whitham, Ames, IA (US); Yu Mei, Ames, IA (US)

(21) Appl. No.: 15/411,045

(22) Filed: Jan. 20, 2017

Related U.S. Application Data
(60) Provisional application No. 62/286,058, filed on Jan. 22, 2016.

(51) Int. Cl. C12N 15/82; C07K 14/005; C12N 7/00

(52) U.S. Cl. CPC C12N 15/8218; C12N 15/8274; C12N 15/8213; C12N 15/8207; C12N 15/8206; C12N 7/00; C07K 14/005; C12N 2770/00043; C12N 2770/00021; C12N 2770/00031

(57) ABSTRACT

The present invention provides plant virus vectors developed from the Foxtail mosaic virus (FoMV). The vectors include a nucleic acid sequence encoding an infectious Foxtail mosaic virus (FoMV) with a functional movement encoding sequence operably linked to one or more regulatory elements functional in a plant. The plant virus vectors may be used to infect monocot plants, such as maize and can be used for VIGS, gene editing, gene expression or other transgenic protocols.

### CROSS-REFERENCE TO RELATED APPLICATIONS

This application claims priority under 35 U.S.C. §119 to provisional application Ser. No. 62/286,058, filed Jan. 22, 2016, herein incorporated by reference in its entirety.

### FIELD OF THE INVENTION

The present invention relates to methods for genetic manipulation of monocot plants. More specifically, the invention describes the use of a Foxtail mosaic virus based vector designed for gene silencing and foreign gene expression applications.

### BACKGROUND OF THE INVENTION

Plant virus-based vectors for expressing heterologous proteins in plants present promising biotechnological tools to supplement conventional breeding and transgenic technologies. Considering the speed with which a virus infection becomes established throughout a plant and the high yield of viral-encoded proteins that accumulate in plants, the use of viral vectors provides an attractive and cost effective means for the production of valuable proteins in plants and for rapid evaluation of new traits. Due to these advantages, many viral vectors have been developed and used, especially in dicot plants (Caplan and Dinesh-Kumar, 2006; Purkayastha and Dasgupta, 2009; Senthil-Kumar and Mysore, 2011).

Plant virus-based vectors have been developed to express heterologous proteins in plants for the study of gene function, production of pharmaceuticals, analysis of plant-microbe interactions, fungicide and insecticide screening, metabolic engineering and nutrient improvement and represent valuable means to supplement conventional breeding and transgenic technology.

Plant viral vectors can be also used as virus-induced gene silencing (VIGS) reverse genetics tools to reduce or inhibit gene function. VIGS is a type of RNA silencing that is initiated by recombinant virus vectors carrying fragments of host genes being silenced. The plants are infected with the recombinant viruses to activate the RNA silencing of endogenous target gene of host plants. VIGS is an efficient and reliable method though there are many techniques being conducted to induce gene silencing. Gene transformation is not required in the VIGS process, which provides a quick and feasible way for plants with lengthy life cycle and transformation difficulties.

VIGS can specifically down regulate a single gene, members of a gene family, or sets of distinct genes, without requiring introduction of a full length gene. It was reported that 23 nucleotides was enough to induce gene silencing (Plant J. 25, 417-25, 2001). The use of small fragments alleviates problems of acquiring the whole cDNA and can enhance the specificity of VIGS. Compared with transformation of plants with sense and/or antisense gene approaches, the advantage of VIGS is the relative speed. Moreover it suppresses the target gene RNA level after the seedling is established, which allows the functions of the essential genes to be tested upon silencing.

Despite the fact that many viral vectors have been developed and used in dicot plants (Caplan and Dinesh-Kumar, 2006; Purkayastha and Dasgupta, 2009; Zhang et al., 2009; Senthil-Kumar and Mysore, 2011), relatively few have been developed for monocots. Seven different viruses have been developed into viral vectors for monocots to date (Lee et al., 2015). These include Barley stripe mosaic virus (BSMV) (Scofield et al., 2005; Lee et al., 2012), Brome mosaic virus (BMV) (Ding et al., 2006), Cymbidium mosaic virus (CymMV) (Lu et al., 2007), Rice tungro bacilliform virus (RTBV) (Purkayastha et al., 2010), Wheat streak mosaic virus (WSMV) (Choi et al., 2000; Tatineni et al., 2011), Bamboo mosaic virus (BaMV) together with its associated satellite RNA (Liou et al., 2014), and Cucumber mosaic virus (CMV) (Wang et al., 2016). Six of them are designed for VIGS applications (BSMV, BMV, CymMV, RTBV, BaMV, and CMV) and only two of them can be used for systemic gene expression (BSMV and WSMV). The BMV vector has been shown to have the ability to infect maize (Ding et al. 2006; Lee et al., 2015). However, there have been relatively few studies published on its applications for VIGS suggesting that it has not been widely adopted in the maize research community (Ding et al., 2006; van der Linde et al., 2011; van der Linde and Doehlemann, 2013). Although WSMV-mediated gene expression has been recorded in maize (Choi et al., 2000; Tatineni et al., 2011), there has been no further application of WSMV vectors in maize research.

It is an object of the present invention to disclose infectious full-length plant virus vector system based on Foxtail mosaic virus (FoMV) for gene silencing (e.g., VIGS or CRISPR) and foreign gene expression in maize and other monocots.

### SUMMARY OF THE INVENTION

The present invention provides plant virus vectors developed from the Foxtail mosaic virus (FoMV). According to the invention, the vector includes a nucleic acid construct with an infectious FoMV sequence operably linked to regulatory sequences functional in a plant cell. The plant virus vectors may be used to infect monocot plants, such as maize.

The FoMV vectors according to the invention may be full length, variant or truncated FoMV sequences but will include functional coat protein, and movement sequences (triple gene block sequences ORF 2, 3, and 4). This is in distinction from other FoMV based plant virus vectors, which include a FoMV virus sequence that is noninfectious and includes inactivated capsid protein and/or inactivation of one more triple block protein encoding regions. The FoMV sequence is engineered to include a heterologous restriction site for insertion of sequences.

In one embodiment optimized for use with VIGS, the restriction sites are introduced after the stop codon of the capsid protein encoded by open reading frame number 5. The present invention also relates to a method of silencing or inhibiting expression of an endogenous sequence of interest in plants. More specifically, the present invention relates to a method of inhibiting expression of one or more target genes, promoters or other endogenous sequence in plants, particularly monocot plants such as maize. According to the invention, a silencing sequence is introduced to said polynucleotide vector construct. Typically the silencer sequence comprises an endogenous target sequence that is introduced to the viral construct in reverse orientation and is of sufficient length that upon expression of the same, mRNA is bound and activity of the endogenous sequence is inhibited.

In another embodiment optimized for foreign gene expression, the vector is engineered to lack the dispensable ORF5A and contains a duplication of the subgenomic promoter for capsid protein followed by a heterologous restriction site for insertion of sequences. In a further embodiment, codons in the area that overlaps with ORF4 are changed in a way that the derived amino acid is not altered in order to minimize homologous recombination and instability.

The present invention also relates to a method of expressing foreign genes of interest in plants, including monocots such as maize. According to the invention, a foreign sequence is introduced to said polynucleotide vector construct. It is a further objective to provide a method for guide polynucleotide delivery to facilitate targeted genome editing with endonucleases such as TALENS or CRISPR-Cas in plant species susceptible to FoMV.

In some embodiments, vectors are DNA-based and designed to be delivered directly into plant cells by biolistic inoculation. It is yet a further object of the invention to provide a FoMV-based vector compatible with agroinoculation.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 shows schematic representation of the Foxtail mosaic virus (FoMV) infectious clone (pFoMV-IA) with a multiple cloning site (MCS) for insertion of plant gene fragments for silencing (pFoMV-V). The MCS containing the XbaI and XhoI restriction enzyme sites was placed after the stop codon of open reading frame 5 (ORF5), which encodes the coat protein. ORF1 encodes the RNA dependent RNA polymerase and is required for replication. ORFs 2, 3, and 4 encode the triple gene block proteins required for movement. ORF5A has unknown function and may be dispensable. The viral sub-genomic RNAs (sgRNA1 and sgRNA2) used to express the triple gene block proteins and coat protein, respectively, are indicated by the gray bars. The Cauliflower mosaic virus 35S promoter (P35S) is fused to the 5′ end of the FoMV genomic RNA in order to initiate synthesis of genome-length RNA transcripts in plant cells. The viral genomic RNA terminates with a tract of A residues (Poly (A) tail), and it is followed by the nopaline synthase terminator (Tnos) in the infectious clones.

FIGS. 2A-2B show infection of sweet corn (Golden×Bantam) by the Foxtail mosaic virus (FoMV) infectious clones.

FIG. 2A shows leaf images from control and inoculated plants from left to right: non-inoculated (NI), mock-inoculated (Mock), FoMV infectious clone (pFoMV-IA), and FoMV infectious clone carrying the empty cloning site (pFoMV-V). Bar=1 cm.

FIG. 2B shows an RT-PCR assay to detect presence of FoMV in systemic leaf tissues. From left to right: non-inoculated (NI), mock-inoculated (Mock), pFoMV-IA, and leaf 6 (L6), leaf 9 (L9), and the top leaf (L top, usually leaf 13, but occasionally 12 or 14) of pFoMV-V inoculated plants. The 295 bp or 318 bp FoMV fragment is detected in plants inoculated with pFoMV-IA or pFoMV-V, respectively. Maize actin was included as internal control for RT-PCR.

FIGS. 3A-3B show virus-induced gene silencing of the maize pytoene desaturase (pds) gene using the FoMV vector.

FIG. 3A shows sweet corn (Golden×Bantam) plants that were biolistically inoculated with the pFoMV-V (empty vector) and pFoMV-PDS (carries a 313 bp fragment of maize pds) infectious clones. From left to right: Images of the fifth leaves from plants that were mock-inoculated (Mock) or infected by FoMV-V and FoMV-PDS. The leaf on the right shows streaks of photo-bleached tissue caused by pds silencing.

FIG. 3B shows the photo-bleaching phenotype caused by pds silencing in systemic leaves of plants that were rub-inoculated with sap from FoMV-PDS infected tissue. The photo-bleaching phenotype is shown for leaf 4 (L4) and leaf 5 (L5). Bar=1 cm.

FIG. 4 shows quantitative real-time RT-PCR analysis of pds expression in non-infected (NI), FoMV-V empty vector (EV), and FoMV-PDS-infected sweet corn (Golden×Bantam) plants. Significant suppression of pds mRNA transcripts is detected in systemic leaves of plants that were biolistically inoculated with pFoMV-PDS (* indicates P<0.05 compared to EV). B1, B2, B3 indicates the fourth leaf of three different biolistically inoculated plants); AS1, AS2, AS3 indicates asymptomatic systemic leaves on three different biolistically inoculated plants. The pds silencing effect is also observed in rub-inoculated plants indicating that the FoMV-PDS can be passaged at least one time. R1-L4, R1-L5, R2-L4, R2-L5 indicates leaves four and five on two different rub-inoculated plants.

FIG. 5 shows infection course analysis of the stability of the pds gene fragment and pds mRNA silencing following biolistic or rub inoculation of FoMV-PDS in sweet corn plants (Golden×Bantam). The inset gel images show the RT-PCR analyses for the pds insert stability in FoMV-PDS infected plants. The upper gel image is the RT-PCR control showing amplification of a single maize actin mRNA fragment in all samples. The lower gel image is RT-PCR amplification across the FoMV cloning site. EV indicates the FoMV-V empty vector that carries no insert. L4, L6, L9, L top indicate the leaf number that the sample was taken from. The bar graphs display the relative expression level of pds mRNA in the indicated leaves determined by quantitative real-time RT-PCR. B# indicates the independent plants that were biolistically inoculated and the R# indicates the independent plants that were rub-inoculated. The bottom-most graph shows the relative expression of pds in each leaf sample type averaged over all plants. The error bars indicate standard deviation of three technical replicates for each individual plant, except for the bottom-most graph in which they indicated the standard deviation of the mean for all plants.

FIGS. 6A-6B show silencing les22 using the FoMV VIGS vector in sweet corn (Golden×Bantam).

FIG. 6A shows from left to right: mock inoculated (Mock); FoMV-V empty vector; FoMV vector carrying a 330 bp fragment of les22. Bar=1 cm.

FIG. 6B shows show close-up views of sections of the same sweet corn leaves that are shown in A in the upper panels. The lower panels show the leaves after staining with trypan blue. Bar=0.5 cm.

FIG. 7 shows infection course analysis of the stability of the les22 gene fragment and les22 mRNA silencing following biolistic or rub inoculation of FoMV-Les22 in sweet corn plants (Golden×Bantam). The inset gel images show the RT-PCR analyses for the les22 insert stability in FoMV-Les22-infected plants. The upper gel image is the RT-PCR control showing amplification of a single maize actin mRNA fragment in all samples. The lower gel image is RT-PCR amplification across the FoMV cloning site. EV indicates the FoMV-V empty vector that carries no insert. L4, L6, L9, L top indicate the leaf number that the sample was taken from. The bar graphs display the relative expression level of les22 mRNA in the indicated leaves determined by quantitative real-time RT-PCR B# indicates the independent plants that were biolistically inoculated and the R# indicates the independent plants that were rub-inoculated. The bottom-right graph shows the relative expression of les22 in each leaf sample type averaged over all plants. The error bars indicate standard deviation of three technical replicates for each individual plant, except for the bottom-right graph in which they indicate the standard deviation of the mean for all plants.

FIGS. 8A-8B show FoMV infection of maize inbred lines, sorghum, and green foxtail (Setaria viridis).

FIG. 8A shows mosaic symptoms caused by FoMV-V infection observed on systemic leaves of maize inbred lines (B73, B101, W22CC, K55, FR1064, B104, A188 and W64A), sorghum, and green foxtail, but not on maize inbred lines Mo47 or Mo17. Bar=1 cm.

FIG. 8B shows RT-PCR amplification of a FoMV-specific PCR product confirming FoMV infection in maize inbred lines B73, B101, W22CC, K55, FR1064, B104, A188, W64A and Mo47, and also in sorghum and green foxtail, but not in maize inbred line Mo17. (Sweet corn samples infected with FoMV are included as positive controls). The FoMV genomic fragment can only be detected in plants inoculated with FoMV-V but not in mock-treated plants. The actin from maize, sorghum or green foxtail was included as internal control.

FIGS. 9A-9B show analysis of the stability of the bm3 gene fragment in FoMV-Bm3 and bm3 mRNA silencing following biolistic inoculation of sweet corn plants (Golden×Bantam).

FIG. 9A shows RT-PCR analysis of FoMV empty vector (EV) and pFoMV-Bm3-infected sweet corn plants. Maize actin is shown in the upper panel and the FoMV-specific fragment is shown in the lower panel. Larger bands of FoMV fragments detected in pFoMV-Bm3 infected plants indicate the insertion of the bm3 fragment in the viral genome. Numbers above the lanes indicate individual plants.

FIG. 9B shows quantitative real-time RT-PCR analysis of bm3 expression in FoMV-V empty vector (EV), and FoMV-Bm3-infected sweet corn plants. Significant suppression of bm3 mRNA transcripts is detected in pFoMV-Bm3 infected leaves from five independent plants (Bm3-1, 2, 3, 4, 5) (* indicates P<0.05 compared to EV).

FIGS. 10A-10B show virus-induced gene silencing of the maize ij gene using the FoMV vector.

FIG. 10A shows sweet corn (Golden×Bantam) plants were mock treated (left) or biolistically inoculated with pFoMV-Ij (carries a 231 bp fragment of maize ij) infectious clone (right). The leaf on the right (Leaf 6) shows white stripes caused by ij silencing. White stripe at the leaf margin is highlighted in red box. Bar=1 cm.

FIG. 10B shows quantitative real-time RT-PCR analysis of ij expression in FoMV-V empty vector (EV), and FoMV-Ij-infected sweet corn (Golden×Bantam) plants. Significant suppression of ij mRNA transcripts is detected in systemic leaves of plants that were biolistically inoculated with pFoMV-Ij (* indicates P<0.05 compared to EV). B1, B2, B3, B4 and B5 indicate the sixth leaf of five different biolistically inoculated plants).

FIGS. 11A-11C show the FoMV viral sequences.

FIG. 11A shows the full length FoMV sequence including MCS (SEQ ID NO: 1). ORF1: 81-4088 (SEQ ID NO: 7); ORF2: 4132-4842 (SEQ ID NO: 8); ORF3: 4775-5158 (SEQ ID NO: 9); ORF4:5140-5298 (SEQ ID NO: 10); ORF5A: 5228-6028 (SEQ ID NO: 11); ORF5: 5372-6028 (SEQ ID NO: 12).

FIG. 11B-11C show the sequence of the pFoMV-V silencing vector (SEQ ID NO: 2): 35S promoter is yellow shaded (SEQ ID NO: 3); NOS terminator is green shaded (SEQ ID NO: 4); XbaI is pink shaded (SEQ ID NO: 5); XhoI is Turquoise shaded (SEQ ID NO: 6); Full length FoMV sequence including MCS is in italics (SEQ ID NO: 1)

FIGS. 12A-12C show schematic and sequence information of FoMV based viral vectors.

FIG. 12A shows a schematic representation of the Foxtail mosaic virus (FoMV) silencing vector (pFoMV-V). The MCS I contains the XbaI and XhoI restriction enzyme sites.

FIG. 12B shows a schematic representation of the FoMV expression vectors (pFoMV-DP/DC). The MCS II contains the Bsu36I, HpaI and PspOMI restriction enzyme sites.

FIG. 12C shows the sequence between ORF4 and ORF5 in the pFoMV-DP (SEQ ID NO: 20) and pFoMV-DC (SEQ ID NO: 21) vectors. The duplicated subgenomic promoter and MCS II are in bold. The codon change in pFoMV-DC vector is highlighted in red.

FIGS. 13A-13B show infection of sweet corn (Golden×Bantam) by the Foxtail mosaic virus (FoMV) viral vectors.

FIG. 13A shows leaf images from control and inoculated plants from left to right: non-inoculated (NI), FoMV silencing vector (pFoMV-V), pFoMV-V with mutated ORF5A (pFoMV-V-Δ5A), FoMV expression vector pFoMV-DP and FoMV expression vector pFoMV-DC. Bar=1 cm.

FIG. 13B shows RT-PCR assay to detect presence of FoMV in systemic leaf tissues of the plants showed in A. Maize actin was included as internal control for RT-PCR.

FIGS. 14A-14F show FoMV mediated BAR expression in sweet corn (Golden×Bantam).

FIG. 14A shows sweet corn infected by pFoMV-DP-BAR (DP BAR, leaf) and by pFoMV-DC-BAR (DC BAR, right).

FIG. 14B shows RT-PCR analyses for the BAR insert stability in the 4^{th} leaves of FoMV-DP-BAR infected plants.

FIG. 14C shows RT-PCR analyses for the BAR insert stability in the 4^{th} leaves of FoMV-DC-BAR infected plants.

FIG. 14D shows RT-PCR analyses for the BAR insert stability in the 6^{th} leaves of FoMV-DP-BAR infected plants.

FIG. 14E shows RT-PCR analyses for the BAR insert stability in the 6^{th} leaves of FoMV-DC-BAR infected plants. The upper gel images in B, C, D, E are the RT-PCR control showing amplification of a single maize actin mRNA fragment in all samples. The lower gel images are RT-PCR amplification across the FoMV cloning site (MSCII). DP and DC indicate the FoMV-DP and FoMV-DC empty vectors that carry no insert.

FIG. 14F shows strip test for the expression of BAR protein. Positive signals highlighted by red arrow are detected only in FoMV-DC-BAR infected leaf tissue, but not in non-infected (NI) plant or plant infected by pFoMV-DC (DC) control. The red stars in panel C indicate the plants that are used in the strip test for BAR expression.

FIGS. 15A-15C show BAR expression mediated by FoMV protects sweet corn (Golden Bantam) plants from herbicide treatment.

FIG. 15A shows sweet corn plants before (upper panels) and after (lower panels) treatment of FINALI herbicide. From left to right: non-inoculated (NI), plants infected by pFoMV-DC (DC), pFoMV-DP (DP), pFoMV-DC-BAR (DC BAR) and pFoMV-DP-BAR (DP BAR).

FIG. 15B shows individual DC BAR plants after herbicide treatment.

FIG. 15C shows a representative image of DP BAR plant after herbicide treatment. Red stars in B and C indicate the 4^{th} leaves.

FIG. 16 shows sweet corn plants before (upper panels) and after (lower panels) treatment of FINALI herbicide starting at 23 DPI. From left to right: non-inoculated (NI), plants infected by pFoMV-DC-BAR (DC BAR) and pFoMV-DP-BAR (DP BAR).

FIGS. 17A-17B show sweet corn plants before (upper panels) and after (lower panels) treatment of FINALI herbicide starting at 13 DPI.

FIG. 17A shows from left to right: non-inoculated (NI), mock treated (Mock), plants rub-inoculated by FoMV-DC (DC) and plants rub-inoculated by FoMV-DC-BAR (DC BAR).

FIG. 17B shows representative images of DC BAR rub-inoculated plants after herbicide treatment. From left to right: green, partial green and yellow plant.

FIGS. 18A-18D shows FoMV mediated GFP expression in sweet corn (Golden Bantam).

FIG. 18A shows sweet corn infected by pFoMV-DC (DC, left) and by pFoMV-DC-GFP (DC GFP, right).

FIG. 18B shows RT-PCR analyses for the GFP insert stability in FoMV-DC-GFP infected plants. The upper gel image is the RT-PCR control showing amplification of a single maize actin mRNA fragment in all samples. The lower gel image is RT-PCR amplification across the FoMV cloning site MSCII. DC indicates the FoMV-DC empty vector that carries no insert. L4, L6, L9 indicate the leaf number that the sample was taken from.

FIG. 18C shows western blot of GFP expression in FoMV-DC-GFP infected leaf tissues that are used in panel B. The upper images are western results using anti-GFP antibody and the lower images show the protein loading control.

FIG. 18D shows green fluorescence in the 4^{th} leaf of DC or DC GFP infected sweet corn plants. Area in the red box is enlarged at the bottom using different focus.

FIG. 19 shows green fluorescence observation in DC GFP infected sweet corn leaves. L4, L6, L7 and L8 indicate the leaf number that the sample was taken from.

FIGS. 20A-20C show using FoMV vector for gRNA delivery.

FIG. 20A shows a schematic representation of FoMV-derived VIGS vector pFoMV-V carrying a 20-bp target sequence specific to Sish1 or CA2 followed by the 84-bp scaffold sequence for Cas9 binding.

FIG. 20B shows a schematic representation of FoMV-derived expression vector pFoMV-DP/DC carrying a 20-bp target sequence specific to Sish1 or CA2 followed by the 84-bp scaffold sequence for Cas9 binding.

FIG. 20C shows RT-PCR analyses for the insert stability in FoMV-DP-CA2 infected plants. DP indicates the FoMV-DP empty vector that carries no insert. L4, L6, L9 and Ltop indicate the leaf number that the sample was taken from.

FIGS. 21A-21D show the FoMV vector compatible with Agroinoculation.

FIG. 21A shows schematic representation of FoMV vector in pCAMBIA1380 binary vector.

FIG. 21B shows systemic leaf of Nicotiana benthamiana (N.B.) plants that are non-inoculated (NI) or Agro-infiltrated by agrobacteria carrying pFoMV-Agro. Bar=1 cm.

FIG. 21C shows sweet corn leaf of mock treated plant or plant that is rub-inoculated with infected N.B. tissue. Bar=1 cm.

FIG. 21D shows western blot of FoMV infection in N.B. and sweet corn leaf tissues that are showed in panel B and C. − indicates NI or mock treated; + indicates leaf with viral symptoms.

FIGS. 22A-22H show FoMV viral sequences.

FIG. 22A shows the full length FoMV sequence including MCS (SEQ ID NO: 1). ORF1: 81-4088 (SEQ ID NO: 7); ORF2: 4132-4842 (SEQ ID NO: 8); ORF3: 4775-5158 (SEQ ID NO: 9); ORF4:5140-5298 (SEQ ID NO: 10); ORF5A: 5228-6028 (SEQ ID NO: 11); ORF5: 5372-6028 (SEQ ID NO: 12).

FIG. 22B-22C show the sequence of the pFoMV-DP expression vector (SEQ ID NO: 13): 35S promoter is yellow shaded (SEQ ID NO: 3); NOS terminator is green shaded (SEQ ID NO: 4); XbaI is pink shaded (SEQ ID NO: 5); XhoI is Turquoise shaded (SEQ ID NO: 6); Full length FoMV sequence including MCS is in italics (SEQ ID NO: 1); MCSII including Bsu36I, HpaI and PspOMI is gray shaded (SEQ ID No: 14); The duplication of the putative CP promoter (DP) is in bold (SEQ ID NO: 15); Full length FoMV sequence including MCS is in italics (SEQ ID NO: 1).

FIG. 22D-22E show the sequence of the pFoMV-DC expression vector (SEQ ID NO: 16): 35S promoter is yellow shaded (SEQ ID NO: 3); NOS terminator is green shaded (SEQ ID NO: 4); XbaI is pink shaded (SEQ ID NO: 5); XhoI is Turquoise shaded (SEQ ID NO: 6); MCSII including Bsu36I, HpaI and PspOMI is gray shaded (SEQ ID No: 14); The duplication of the putative CP promoter with codon change (DC) is in bold and the changed nucleotide is highlighted with red (SEQ ID NO: 17); Full length FoMV sequence including MCS is in italics (SEQ ID NO: 1).

FIG. 22F-22H show the sequence of the pCAMBIA1380-FoMV vector (SEQ ID NO: 18).

pCAMBIA1380 sequence is in lowercase (SEQ ID NO: 19); 35S promoter is yellow shaded (SEQ ID NO: 3); NOS terminator is green shaded (SEQ ID NO: 4); XbaI is pink shaded (SEQ ID NO: 5); PacI is olive shaded; MCSII including Bsu36I, HpaI and PspOMI is gray shaded (SEQ ID No: 14); The duplication of the putative CP promoter with codon change (DC) is in bold and the changed nucleotide is highlighted with red (SEQ ID NO: 17); Full length FoMV sequence including MCS is in italics (SEQ ID NO: 1).

FIGS. 23A-23C show maps of the viral vectors.

FIG. 23A shows a map of the pFoMV-V vector.

FIG. 23B shows a map of the pFoMV-DP vector.

FIG. 23C shows a map of the pFoMV-DC vector.

### DETAILED DESCRIPTION OF THE INVENTION

The ability of Foxtail mosaic virus (FoMV) to infect maize and other monocots makes it a candidate for viral vector development (Paulsen and Niblett, 1977). FoMV is a member of the genus Potexvirus, which is a large group of flexuous and filamentous plant viruses with a single-stranded, positive-sense genomic RNA. The full length genomic sequence of FoMV was first reported in 1991 (Bancroft et al., 1991). Later, an RNA-based full-length infectious clone was constructed and a revised genome was published (Robertson et al., 2001; Bruun-Rasmussen et al., 2008). The genome structure of FoMV is similar to that of Potato virus X (PVX), which is the type member of the potexviruses. They both contain five major open reading frames (ORF) (Huisman et al., 1988; Robertson et al., 2001; Bruun-Rasmussen et al., 2008). ORF1 encodes the RNA dependent RNA polymerase (RdRp), which is necessary for viral RNA replication and subgenomic messenger RNA (sgRNA) synthesis (Draghici et al., 2009). The overlapping ORFs 2, 3, and 4 are known as the triple gene block (TGB), which functions in virus movement and suppression of host defense (Verchot-Lubicz, 2005). The ORF5 encodes the coat protein (CP), that is indispensable for virus assembly and cell-to-cell movement (Cruz et al., 1998). In addition to the five ORFs, the FoMV genome has a unique ORF5A that initiates 143 nucleotides upstream of the CP. Although the 5A protein is produced in vivo, it is considered to be dispensable because it is not required for replication or for systemic infection of plants (Robertson et al., 2000).

PVX can be used for both VIGS and gene expression. In those PVX vectors, foreign genes or DNA fragments for VIGS are inserted between ORF4 and ORF5 under the control of either a duplicated, native CP promoter or a heterologous CP promoter from a related Potexvirus (Sablowski et al., 1995; Lacomme and Chapman, 2008; Dickmeis et al., 2014; Wang et al., 2014). The PVX vectors with duplicated promoters frequently suffer partial or complete loss of inserted sequences, especially when the insert size is large or the recombinant virus is passaged (Avesani et al., 2007; Dickmeis et al., 2014). In a PVX vector study involving heterologous sub-genomic promoter-like sequences, a Bamboo mosaic virus (BaMV) sub-genomic promoter combined with an N-terminal CP deletion resulted in the highest stability of foreign inserts following a passage to new plants (Dickmeis et al., 2014).

Disarmed FoMV vectors that cannot spread systemically have been developed for gene expression previously. However, this set of expression vectors is designed for restricted local gene expression via Agrobacterium infiltration-mediated delivery (Liu and Keamey 2010). This FoMV vector system cannot systemically infect plants, because it lacks necessary movement functions encoded by the TGB and CP. Here, Applicants aimed to develop a reliable FoMV vector system that is able to establish whole plant systemic infection, carry foreign gene insertions homologous to endogenous host genes targeted for efficient silencing, and is capable of expressing foreign genes in systemically infected leaves.

The FoMV viral vector system has the potential to provide a powerful biotechnological tool needed to supplement conventional genetics and transgenic plant approaches for identifying gene functions. FoMV has a broad host range including 56 species of Gramineae (e.g. maize, sorghum, rice, barley, green foxtail) and at least 35 dicot species (e.g. soybean and tobacco) (Paulsen and Niblett, 1977). We confirmed that at least three maize inbred lines, sorghum, and green foxtail can be infected by our FoMV isolate. Thus, the successful development of this FoMV viral vector for maize is expected to readily translate into a useful functional genomics platform for research and improvement in other monocot plants of economic and scientific interest. The FoMV expression vector reported here provides a useful platform to supplement conventional genetics and transgenic plant approaches for identifying gene functions in maize, especially when used together with the FoMV based VIGS vectors. It is also expected to be easily transferred for research and improvement in other monocot plants, such as sorghum, barley, wheat and foxtail millet.

Many modifications and other embodiments of the invention will come to mind to one skilled in the art to which this invention pertains, having the benefit of the teachings presented in the descriptions and the drawings herein. As a result, it is to be understood that the invention is not to be limited to the specific embodiments disclosed and that modifications and other embodiments are intended to be included within the scope of the appended claims. Although specific terms are used in the specification, they are used in a generic and descriptive sense only and not for purposes of limitation.

### General

In order to provide a clear and consistent understanding of the specification and the claims, including the scope given to such terms, the following definitions are provided. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989: 3d ed., 2001: Ausubel et al., CURRENT PROTOCOLS IN MOLECULAR BIOLOGY, John Wiley & Sons, New York, 1987 and periodic updates: the series METHODS IN ENZYMOLOGY, Academic Press, San Diego; Wolffe. CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119. “Chromatin Protocols” (P. B. Becker, ed.) Humana Press, Totowa, 1999.

### Definitions

The following definitions are provided to facilitate an understanding of the present invention.

Unless otherwise specified, “a,” “an,” “the,” and “at least one” are used interchangeably and mean one or more than one.

“Plant” species of interest include, but are not limited to, corn (Zea mays), soybean (Glycine max), common bean (Phaseolus vulgaris), Peanuts (Arachis hypogaea), Medicago sativa, Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum)), proso millet (Panicum miliaceum), foxtail millet (Setaria italica), finger millet (Eleusine coracana)), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In a preferred embodiment the plant is a monocot plant such as maize. The skilled person will appreciate that the tropism of the viral vectors disclosed herein varies. However, determining susceptibility to such viruses is well within the purview of the skilled person. “Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, plastid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” refers to a nucleic acid segment that may possess transcriptional and translational control sequences, such as promoters, enhancers, translational start signals (e.g., ATG or AUG codons), polyadenylation signals, terminators, and the like, and which facilitate the expression of a nucleic acid sequence in a host cell or organism.

By “host cell” is meant a cell which contains a vector of the present invention and supports the replication and/or expression of said vector. Host cells may be prokaryotic cells such as E. coli, or eukaryotic cells such as yeast, insect, amphibian, or mammalian cells. Preferably, host cells are monocotyledonous, although dicotyledonous plant cells are encompassed as well. A particularly preferred monocotyledonous host cell is a maize host cell.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “introduce”, shall refer to any method or means by which a nucleic acid is facilitated into a cell or host organism and may be used interchangeably to convey the same meaning. Such methods include, but are not limited to, transfection, electroporation, microinjection, PEG-fusion, Agrobacterium infection, and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant. A number of “selectable marker genes” are known in the art and several antibiotic resistance markers satisfy these criteria, including those resistant to kanamycin (nptII), hygromycin B (aph IV) and gentamycin (aac3 and aacC4). Useful dominant selectable marker genes include genes encoding antibiotic resistance genes (e.g., resistance to hygromycin, kanamycin, bleomycin, G418, streptomycin or spectinomycin); and herbicide resistance genes (e.g., phosphinothricin acetyltransferase). A useful strategy for selection of transformants for herbicide resistance is described, e.g., in Vasil, Cell Culture and Somatic Cell Genetics of Plants, Vols. I III, Laboratory Procedures and Their Applications Academic Press, New York, 1984. Particularly preferred selectable marker genes for use in the present invention would be genes which confer resistance to compounds such as antibiotics like kanamycin, and herbicides like glyphosate (Della-Cioppa et al., Bio/Technology 5(6), 1987, U.S. Pat. Nos. 5,463,175, 5,633,435). Other selection devices can also be implemented and would still fall within the scope of the present invention.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

“Native” refers to a naturally occurring (“wild-type”) nucleic acid sequence.

“Heterologous” sequence refers to a sequence which originates from a foreign source or species or, if from the same source, is modified from its original form.

As used herein, the term “endogenous,” when used in reference to a polypeptide, nucleic acid or gene, refers to a polypeptide, nucleic acid or gene that is expressed by a host or already present within a host plant. For example, using a FoMV vector of the invention for a method of virus-induced gene silencing, a FoMV vector is engineered to express at least a portion of a nucleic acid endogenous to the host plant such as maize.

A “coding sequence” or “coding region” refers to a nucleic acid molecule having sequence information necessary to produce a gene product, when the sequence is expressed.

“Genetic component” refers to any nucleic acid sequence or genetic element which may also be a component or part of an expression vector. Examples of genetic components include, but are not limited to promoter regions, 5′ untranslated leaders or promoters, introns, genes, 3′ untranslated regions or terminators, and other regulatory sequences or sequences which affect transcription or translation of one or more nucleic acid sequences.

“Complementary” refers to the natural association of nucleic acid sequences by base-pairing (A-G-T pairs with the complementary sequence T-C-A). Complementarity between two single-stranded molecules may be partial, if only some of the nucleic acids pair are complementary; or complete, if all bases pair are complementary. The degree of complementarity affects the efficiency and strength of hybridization and amplification reactions.

“Homology” refers to the level of similarity between nucleic acid or amino acid sequences in terms of percent nucleotide or amino acid positional identity, respectively, i.e., sequence similarity or identity. Homology also refers to the concept of similar functional properties among different nucleic acids or proteins. “Reduced gene expression” means that the expression of a plant endogenous sequence is reduced in a genetically modified plant cell or genetically modified plant containing a nucleic acid silencer molecule stably integrated in its genome when compared to a plant cell or plant which does not contain the nucleic acid silencer molecule. “Reduced gene expression” may involve a reduction of expression of a plant endogenous nucleic acid by at least 10%, 20%, 30%, 40%, 50%, 60%, 70%, 75%, 80%, 85%, 90%, 95% or 100%.

“Promoter” refers to a nucleic acid sequence located upstream or 5′ to a translational start codon of an open reading frame (or protein-coding region) of a gene and that is involved in recognition and binding of RNA polymerase II and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, organ, or cell type, a promoter may display “enhanced” expression, i.e., a higher level of expression, in one part (e.g., cell type, tissue, or organ) of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

When fused to heterologous DNA sequences, such promoters typically cause the fused sequence to be transcribed in a manner that is similar to that of the gene sequence with which the promoter is normally associated. Promoter fragments that include regulatory sequences can be added (for example, fused to the 5′ end of, or inserted within, an active promoter having its own partial or complete regulatory sequences (Fluhr et al., Science 232:1106 1112, 1986; Ellis et al., EMBO J. 6:1116, 1987; Strittmatter and Chua, Proc. Nat. Acad. Sci. USA 84:8986 8990, 1987; Poulsen and Chua, Mol. Gen. Genet. 214:16 23, 1988; Comai et al., Plant Mol. Biol. 15:373 381, 1991).

The 3′ non-translated region of the gene constructs of the invention contain a transcriptional terminator, or an element having equivalent function, and, optionally, a polyadenylation signal, which functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (Nos) gene, and (2) plant genes such as the soybean storage protein genes and the small subunit of the ribulose-1,5-bisphosphate carboxylase (ssRUBISCO) gene. An example of another 3′ region is that from the ssRUBISCO E9 gene from pea (European Patent Application 385,962, herein incorporated by reference in its entirety).

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ non-translated regions. RNA polymerase transcribes a coding DNA sequence through a site where polyadenylation occurs.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

As used herein, “transgenic plant” or “genetically modified plant” includes reference to a plant that comprises within its nuclear genome a heterologous polynucleotide. Generally, the heterologous polynucleotide is stably integrated within the nuclear genome such that the polynucleotide is passed on to successive generations. The heterologous polynucleotide may be integrated into the genome alone or as part of a recombinant expression cassette. “Transgenic” or “genetically modified” is used herein to include any cell, cell line, callus, tissue, plant part or plant, the genotype of which has been altered by the presence of heterologous nucleic acid including those initially so altered as well as those created by sexual crosses or asexual propagation from the initial transgenic or genetically modified plant. The term “transgenic” or “genetically modified” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide (e.g., FoMV-cased constructs as described herein). Expression vectors of the present invention permit transcription of a nucleic acid inserted therein.

As used herein, “gene editing,” “gene edited” “genetically edited” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganuclease re-engineered as homing endonucleases. The terms also include the use of transgenic procedures and techniques, including, for example, where the change is relatively small and/or does not introduce DNA from a foreign species. The terms “genetic manipulation” and “genetically manipulated” include gene editing techniques, as well as and/or in addition to other techniques and processes that alter or modify the nucleotide sequence of a gene or gene, or modify or alter the expression of a gene or genes.

As used herein, “VIGS” means virus-induced gene silencing.

As used herein, “viral silencing vector” means a DNA construct comprising (i) a sufficient portion of a viral genome to induce VIGS and (ii) a nucleotide sequence that is similar (i.e., a sequence that has a sufficient percent identity or a sufficient percent complementarity to effect down regulation) to at least a fragment of a target gene, wherein the target gene is down-regulated when the viral silencing vector is introduced into a cell. For example, in order to affect VIGS in a plant, the portion of the viral genome required to affect VIGS may include that portion responsible for viral movement and viral replication in the plant. As is known to those skilled in the art, each virus/host combination should be optimized for producing effective silencing vectors. However, it is to be understood that other optimized vectors can be used and are included within the scope of the applicant's teachings. For example, the silencing vector may include the origin of replication, the genes necessary for replication in a plant cell, and one or more nucleotide sequences with similarity to one or more target genes. The vector may also include those genes necessary for viral movement. The nucleotide sequence that is similar to at least a fragment of a target gene may replace any coding or non-coding region that is nonessential for the present purposes of gene silencing, may be inserted into the vector outside the viral sequences, or may be inserted just downstream of an endogenous viral gene, such that the viral gene and the nucleotide sequence are cotranscribed. The size of the nucleotide sequence that is similar to the target gene may depend on the site of insertion or replacement within the viral genome. Accordingly, there are many ways of producing silencing vectors, as known to those skilled in the art. The vectors of the invention may optionally include other sequences known to those of skill in the art such as marker genes, regulatory elements, terminators, antibiotic resistance genes, and the like.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information World Wide Web at ncbi.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

Production of a genetically modified plant tissue either expressing or inhibiting expression of a gene of interest combines the teachings of the present disclosure with a variety of techniques and expedients known in the art. In most instances, alternate expedients exist for each stage of the overall process. The choice of expedients depends on the variables such as the plasmid vector system chosen for the cloning and introduction of the recombinant DNA molecule, the plant species to be modified, the particular gene of interest, promoter elements and upstream elements used. Persons skilled in the art are able to select and use appropriate alternatives to achieve functionality. Culture conditions for expressing desired genes and cultured cells are known in the art. Also as known in the art, a number of both monocotyledonous and dicotyledonous plant species are transformable and regenerable such that whole plants containing and expressing desired genes under regulatory control of the promoter molecules according to the invention may be obtained. As is known to those of skill in the art, expression in transformed plants may be tissue specific and/or specific to certain developmental stages. Truncated promoter selection and gene selection are other parameters which may be optimized to achieve desired plant expression or inhibition as is known to those of skill in the art and taught herein.

The following is a non-limiting general overview of Molecular biology techniques which may be used in performing the invention

### Regulatory Elements

Exemplary promoters for expression of a nucleic acid sequence in FoMV-based constructs include the CaMV 35S promoter (Odell et al., 1985), CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or R gene complex associated promoters (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. In one embodiment of the invention, the native promoter of an isoflavone biosynthesis sequence is used. In another embodiment, a heterologous sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is envisioned that nucleic acids encoding a polypeptide as provided herein may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

### Terminators

Transformation constructs prepared in accordance with the invention will typically include a 3′ end DNA sequence that acts as a signal to terminate transcription and allow for the poly-adenylation of the mRNA produced by coding sequences operably linked to a promoter. Alternatively, a heterologous 3′ end may enhance the expression of coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

### Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

### Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity.

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154,204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a. β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; or a gene encoding for green fluorescent protein (Sheen et al., 1995; Haseloff et al., 1997; Reichel et al., 1996; Tian et al., 1997; WO 97/41228).

A heterologous nucleotide sequence of the present invention can be provided as its wild-type sequence. Alternatively, a synthetic sequence, such as a “plant-optimized” sequence mentioned above can be employed. A nucleotide sequence having a high degree of homology to these sequences, so that the encoded amino acid sequence remains substantially unchanged, are also contemplated. In particular, sequences at least 80%, more preferably 90%, homologous with an aforementioned nucleotide sequence are contemplated. In one embodiment, the FoMV-based transformation vectors as described herein comprise heterologous nucleic acids encoding genes producing increased agronomic traits (e.g., herbicide resistance, pathogen resistance, drought resistance, increased crop yield, etc.). Genes encoding beneficial agronomic traits are known to those in the art. It should be noted, however, that only that those epitopes of an expressed antigenic protein essential for generating the desiredresponse need be present in the translated molecule. Accordingly, C- and/or N-terminal fragments, including portions of fusion proteins, presenting the essential epitopes are contemplated within the invention. Such fragments can be encoded in a vector construct of the invention or can be generated in vivo or in vitro by post-translation cleavage processes.

### Gene Silencing

The vector is used to silence an endogenous target nucleic acid sequence present in a plant cell. The target sequences for silencing can be designed for the production of short hairpin RNA or silencing RNA against the target nucleic acid, gene, etc. Therefore vectors disclosed herein can include a silencing sequence encoding a gene, cDNA or mRNA of interest, or fragment thereof. The sequence can be 70%, 75%, 80%, 85%, 90%, 95%, 98%, 99%, or 100% identical to the sequence of the endogenous target gene, cDNA or mRNA, or 70%, 75%, 80%, 85%, 90%, 95%, 98%, 99%, or 100% identical to a complement thereof. The sequence is typically introduced to the constructs in reverse orientation.

The target polynucleotide of interest can be a full-length gene, or complement thereof. It can include non-coding regions including, but not limited to 5′ untranslated region, 3′ untranslated region, and one or more introns. The polynucleotide of interest can be a gene's coding region, or complement thereof, for example an mRNA or cDNA.

The polynucleotide can be a fragment of a full-length gene, mRNA, or cDNA. The polynucleotide can include the coding region, one or more introns, 5′ untranslated region, 3′ untranslated region or a combination thereof from a full-length gene. For example, the polynucleotide can at least 10, preferably at least 20, more preferably at least 30, most preferably at least 50 nucleotides of a gene, mRNA, or cDNA of interest. In some embodiments, the polynucleotide includes the first 10, 20, 30, 40, 50, 75, 100, 150, 200, 300, 400, 500, 750, or 1000 nucleotides of a gene or mRNA numbering for the 5′ ATG start site. In some embodiments, the polynucleotide includes 10, 20, 30, 40, 50, 75, 100, 150, 200, 300, 400, 500, 750, or 1000 nucleotides beginning 3′ of the ATG start site. In some embodiments, the polynucleotide includes the last 10, 20, 30, 40, 50, 75, 100, 150, 200, 300, 400, 500, 750, or 1000 nucleotides ending with the 3′ stop codon. In some embodiments the polynucleotide includes the entire transcriptional unit of the gene, mRNA, or cDNA of interest.

In some embodiments the polynucleotide directs formation of tasiRNA against all splice variants of a gene or mRNA of interest by including sequences that are common to all of the splice variants. Likewise, the polynucleotide can direct formation of tasiRNA against related genes or mRNA of interest by including one or more sequences that are similar or related between the two related genes. For example, in some embodiments, the polynucleotide includes a sequence that 80%, 85%, 90%, 95%, 98%, 99%, or 100% identical to a sequence found in at least two different genes or mRNA of interest. The polynucleotide can also be specific for one or more splice variants of a gene when the sequence of the polynucleotide is unique that one or more splice variants.

The possible target genes of the compositions disclosed herein include but are not limited to those discussed below. The genes are generally related to one or more functions or pathways in a cell. It is also possible to target a single gene or mRNA. It is also possible to target more than one gene or mRNA simultaneously. Therefore, in some embodiments, the expression of at least two different target genes is reduced. The target genes can originate from a single group of genes direct to the same or related function or pathway. Alternatively, target genes can originate from genes directed to different or unrelated functions or pathways.

Another important application of plant viral vector systems is in studies on host gene function. With more plant genomic information available, a high throughput tool is required. Virus-induced gene silencing (VIGS) is an exceptional reverse genetics tool that can be employed to generate mutant phenotypes for conveying function to unknown genes. VIGS has many advantages over other methods, for example, it is quick and does not require plant transformation (Burch-Smith et al., 2004). In VIGS systems, viruses are designed to carry partial sequence of known or candidate genes in order to link their function to the mutant phenotype. Replication of the recombinant virus and generation of dsRNA intermediates trigger the RNA-mediated host defense system, resulting in degradation of RNA with sequence identity to the recombinant virus including mRNA of the gene of interest. The targets of VIGS can be a single gene, several members of a gene family, or several distinct genes (Lu et al., EMBO J. 22, 5690-5699 (2003a); Peele, et al., Plant J. 27:357-366 (2001); Tumage, et al., Plant J. 30:107-117 (2002)). Many model host plants including N. benthamiana, tomato, tobacco, Arabidopsis, and cassava have been explored (Burch-Smith, et al., Plant J. 39:734-746 (2004)). With the current abundance of genomic information on maize and other grass species (Stacey, et al., Plant Physiol. 135:59-70 (2004)), it is timely to apply VIGS to maize to enhance knowledge of gene function in such a major grain crop. As disclosed herein, FoMV-based vectors of the invention can be used as a VIGS vector for studies on gene function in plants, for example, in maize.

The invention additionally provides a method for virus-induced gene silencing in a maize plant and vectors useful in a method for virus-induced gene silencing. Such a method can include the step of inoculating a maize plant with Foxtail mosaic virus (FoMV) RNA, wherein the FoMV RNA comprises a nucleic acid sequence encoding at least a portion of a gene endogenous to the maize plant. For virus-induced gene silencing, a partial or entire sequence of an endogenous gene can also be located in the untranslated regions (UTRs) of RNA2, or in RNA1 if the sequence is small enough to be accommodated, as discussed above, since it is the expression of the nucleic acid encoding at least a portion of an endogenous gene that results in gene silencing. For a virus-induced gene silencing vector, the insertion in the UTRs can be facilitated by engineering appropriate restriction sites for insertion of the endogenous gene, so long as the inserted endogenous sequence does not impair viral RNA replication and a sufficient amount of infective FoMV is produced.

The FoMV-based vector is suitable for use as a VIGS vector to study gene function maize. Maize is a major grain crop and an important source of food, feed, and biofuels. It is subject to a wide range of pathogens and VIGS is an ideal reverse genetics tool for maize functional genomics aimed at understanding host-microbe interaction.

It has been reported that gene fragments of 23-80 nt can be sufficient for VIGS induction (Thomas et al., 2001; Burch-Smith et al., 2004; Pflieger et al., 2008). Since the insert size for the FoMV PDS silencing construct in this study is about 300 nt, it is theoretically possible to achieve VIGS of multiple maize genes. This is important because maize has genetic redundancy and genes function in parallel signaling pathways (Blanc and Wolfe 2004; Lawrence and Pikaard 2003; Schnable et al. 2009, Science, 326:1112-1115) making simultaneous testing of different combinations of genes or homologs desirable.

Another amenable feature for multiple gene silencing is that there is no limit on translation requirement for foreign gene insertion with the new FoMV VIGS vector. Further, the interesting finding that the 3′ PDS antisense insertion gave the best silencing phenotype in soybean makes the new FoMV VIGS vector applicable for constructing a cDNA VIGS library because a version of the new FoMV VIGS vector was developed so that directional insertion can be achieved.

It will generally be desirable that vectors provided by the invention be capable of systemic spread in an infected plant. However, such a systemic spread may not be essential for efficient gene silencing. A recombinant vector provided by the invention may or may not therefore include all cis-elements required for vascular movement of the vector or even its cell-to-cell spread. In this manner, modulation of plant gene expression in a collection of plant cells may be more efficiently carried out. Methods for inoculating plants and plant cells with recombinant viral vectors or viral particles are well known to those of skill in the art. Such vectors may, for example, be administered in a solution and may also contain any other desired ingredients including buffers, cis-elements, surfactants, solvents and similar components.

### Plant Transformation Techniques

The transformation of suitable agronomic plant hosts using vectors can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Plants can be transformed by a number of reported procedures (U.S. Pat. No. 5,015,580 to Christou, et al.; U.S. Pat. No. 5,015,944 to Bubash; U.S. Pat. No. 5,024,944 to Collins, et al.; U.S. Pat. No. 5,322,783 to Tomes et al.; U.S. Pat. No. 5,416,011 to Hinchee et al.; U.S. Pat. No. 5,169,770 to Chee et al.). A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. No. 5,231,019 Paszkowski et al.; U.S. Pat. No. 5,472,869 to Krzyzek et al.; U.S. Pat. No. 5,384,253 to Krzyzek et al.), gene gun (U.S. Pat. No. 5,538,877 to Lundquist et al. and U.S. Pat. No. 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. No. 5,004,863 to Umbeck and U.S. Pat. No. 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards et al. Plant Cell Rep. 20: 48-54 (2001); Somleva et al. Crop Science 42: 2080-2087 (2002)). Methods for sugarcane transformation have also been described (Franks & Birch Aust. J. Plant Physiol. 18, 471-480 (1991); WO 2002/037951 to Elliott, Adrian, Ross et al.).

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

A. Agrobacterium-Mediated Transformation

One method for introducing a Foxtail mosaic virus-based expression vector into plants is based on the natural transformation system of Agrobacterium. See, for example, Horsch et al., Science 227: 1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10: 1 (1991). Descriptions of Agrobacterium vector systems and methods for Agrobacterium-mediated gene transfer are provided by Gruber et al., supra. Miki et al., supra, and Moloney et al., Plant Cell Reports 8: 238 (1989). See also, U.S. Pat. No. 5,563,055, (Townsend and Thomas), issued Oct. 8, 1996.

B. Direct Gene Transfer

Several methods of plant transformation, collectively referred to as direct gene transfer, have been developed as an alternative to Agrobacterium-mediated transformation. A generally applicable method of plant transformation is microprojectile-mediated transformation wherein DNA is carried on the surface of microprojectiles measuring 1 to 4 □m. The expression vector of the present invention is introduced into plant tissues with a biolistic device that accelerates the microprojectiles to speeds of 300 to 600 m/s which is sufficient to penetrate plant cell walls and membranes. Sanford et al., Part. Sci. Technol. 5: 27 (1987), Sanford, J. C., Trends Biotech. 6: 299 (1988), Klein et al., Bio/Technology 6: 559-563 (1988), Sanford, J. C., Physiol Plant 79: 206 (1990), Klein et al., Biotechnology 10: 268 (1992). See also U.S. Pat. No. 5,015,580 (Christou, et al), issued May 14, 1991; U.S. Pat. No. 5,322,783 (Tomes, et al.), issued Jun. 21, 1994.

Another method for physical delivery of DNA to plants is sonication of target cells. Zhang et al., Bio/Technology 9: 996 (1991). Alternatively, liposome or spheroplast fusion have been used to introduce expression vectors of the present invention into plants. Deshayes et al., EMBO J., 4: 2731 (1985), Christou et al., Proc Natl. Acad. Sci. U.S.A. 84: 3962 (1987). Direct uptake of DNA into protoplasts using CaCl₂ precipitation, polyvinyl alcohol or poly-L-omithine have also been reported. Hain et al., Mol. Gen. Genet. 199: 161 (1985) and Draper et al., Plant Cell Physiol. 21: 451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4: 1495-1505 (1992) and Spencer et al., Plant Mol. Biol. 24: 51-61 (1994).

Following transformation of target tissues, expression of the above-described selectable marker genes allows for preferential selection of transformed cells, tissues and/or plants, using regeneration and selection methods now well known in the art.

It is often desirable to have the DNA sequence in homozygous state which may require more than one transformation event to create a parental line, requiring transformation with a first and second recombinant DNA molecule both of which encode the same gene product. It is further contemplated in some of the embodiments of the process of the invention that a plant cell be transformed with a recombinant DNA molecule containing at least two DNA sequences or be transformed with more than one recombinant DNA molecule. The DNA sequences or recombinant DNA molecules in such embodiments may be physically linked, by being in the same FoMV-based vector, or physically separate on different vectors. A cell may be simultaneously transformed with more than one vector of the invention provided that each vector has a unique selection marker gene. Alternatively, a cell may be transformed with more than one vector sequentially allowing an intermediate regeneration step after transformation with the first vector. Further, it may be possible to perform a sexual cross between individual plants or plant lines containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques and/or Southern blot techniques known to those of skill in the art.

The transformed cells may then be regenerated into a transgenic plant. The regenerated plants are transferred to standard soil conditions and cultivated in a conventional manner.

After the FoMV-based expression or inhibition cassette is stably incorporated into regenerated transgenic plants, it can be transferred to other plants by sexual crossing. Any of a number of standard breeding techniques can be used, depending upon the species to be crossed.

It may be useful to generate a number of individual transformed plants with any recombinant construct in order to recover plants free from any position effects. It may also be preferable to select plants that contain more than one copy of the introduced recombinant DNA molecule such that high levels of expression of the recombinant molecule are obtained.

As indicated above, it may be desirable to produce plant lines which are homozygous for a particular gene. In some species this is accomplished rather easily by the use of another culture or isolated microspore culture. By using these techniques, it is possible to produce a haploid line that carries the inserted gene and then to double the chromosome number either spontaneously or by the use of colchicine. This gives rise to a plant that is homozygous for the inserted gene, which can be easily assayed for if the inserted gene carries with it a suitable selection marker gene for detection of plants carrying that gene. Alternatively, plants may be self-fertilized, leading to the production of a mixture of seed that consists of, in the simplest case, three types, homozygous (25%), heterozygous (50%) and null (25%) for the inserted gene. Although it is relatively easy to score null plants from those that contain the gene, it is possible in practice to score the homozygous from heterozygous plants by southern blot analysis in which careful attention is paid to the loading of exactly equivalent amounts of DNA from the mixed population, and scoring heterozygotes by the intensity of the signal from a probe specific for the inserted gene. It is advisable to verify the results of the southern blot analysis by allowing each independent transformant to self-fertilize, since additional evidence for homozygosity can be obtained by the simple fact that if the plant was homozygous for the inserted gene, all of the subsequent plants from the selfed seed will contain the gene, while if the plant was heterozygous for the gene, the generation grown from the selfed seed will contain null plants. Therefore, with simple selfing one can easily select homozygous plant lines that can also be confirmed by southern blot analysis.

Creation of homozygous parental lines makes possible the production of hybrid plants and seeds which will contain a modified protein component. Transgenic homozygous parental lines are maintained with each parent containing either the first or second recombinant DNA sequence operably linked to a promoter. Also incorporated in this scheme are the advantages of growing a hybrid crop, including the combining of more valuable traits and hybrid vigor.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

### Integration of a Heterologous Nucleic Acid Insert

Integration of an exogenous nucleic acid insert provided by the FoMV-based expression cassette as described herein can be accomplished by random integration into the plants genome or through site-specific integration. Site-specific integration of an exogenous nucleic acid at a native locus may be accomplished by any technique known to those of skill in the art. In some embodiments, integration of a heterologous nucleic acid insert at a native plant locus comprises contacting a cell (e.g., an isolated cell or a cell in a tissue) with a nucleic acid molecule of the present invention comprising the heterologous nucleic acid insert. In examples, such a nucleic acid molecule may comprise nucleotide sequences flanking the exogenous nucleic acid that facilitate homologous recombination between the nucleic acid molecule and at least one native locus. In particular examples, the nucleotide sequences flanking the exogenous nucleic acid that facilitate homologous recombination may be complementary to endogenous nucleotides of the native locus. In some embodiments, the heterologous nucleic acid insert provides for improved agronomic traits. In some embodiments, a plurality of exogenous nucleic acids may be integrated, such as in gene stacking.

Integration of a nucleic acid may be facilitated (e.g., catalyzed) in some embodiments by endogenous cellular machinery of a host cell, such as, for example and without limitation, endogenous DNA and endogenous recombinase enzymes. In some embodiments, integration of a nucleic acid may be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell via the FoMV-based expression vectors of the present invention. Accordingly, in some examples, a nucleic acid comprising a nucleotide sequence encoding at least one nuclease, recombinase, and/or ligase polypeptide may be introduced into the host cell, either concurrently or sequentially with a nucleic acid to be integrated site-specifically, wherein the at least one nuclease, recombinase, and/or ligase polypeptide is expressed from the nucleotide sequence in the host cell.

### Targeted Endonuclease Systems

Genome editing tools such as transcription activator-like effector nucleases (TALENs) and zinc finger nucleases (ZFNs) have impacted the fields of biotechnology, functional genomic studies in many organisms, and are contemplated to be used with the FoMV-based expression systems describe herein. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

In a preferred embodiment, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Wastson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the foreign nucleic acid. Thus, in the bacterial cell, several Cas proteins are involved with the natural function of the CRISPR/Cas system and serve roles in functions such as insertion of the foreign DNA etc.

Compositions and methods for making and using CRISPR-Cas systems are described in U.S. Pat. No. 8,697,359, entitled “CRISPR-CAS SYSTEMS AND METHODS FOR ALTERING EXPRESSION OF GENE PRODUCTS,” which is incorporated herein in its entirety.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

As used herein, the term “guide polynucleotide”, relates to a polynucleotide sequence that can form a complex with a Cas endonuclease and enables the Cas endonuclease to recognize and optionally cleave a DNA target site (see also U.S. patent application Ser. No. 14/462,691, filed on Aug. 20, 2014, incorporated by reference herein). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

In particular embodiments, the FoMV-based vector comprises a DNA-binding polypeptide or guide RNA that specifically recognizes and binds to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Vector Construction

Construction of vectors for use with the invention will be well known to those of skill in light of the current disclosure. Recombinant constructs preferably comprise restriction endonuclease sites to facilitate vector construction. Particularly useful are unique restriction endonuclease recognition sites. Examples of such restriction sites include sites for the restriction endonucleases HindIII, Tth 1111, BsmI, KpnI and XhoI. Endonucleases preferentially break the internal phosphodiester bonds of polynucleotide chains. They may be relatively unspecific, cutting polynucleotide bonds regardless of the surrounding nucleotide sequence. However, the endonucleases which cleave only a specific nucleotide sequence are called restriction enzymes. Restriction endonucleases generally internally cleave nucleic acid molecules at specific recognition sites, making breaks within “recognition” sequences that in many, but not all, cases exhibit two-fold symmetry around a given point. Such enzymes typically create double-stranded breaks.

Many of these enzymes make a staggered cleavage, yielding DNA fragments with protruding single-stranded 5′ or 3′ termini. Such ends are said to be “sticky” or “cohesive” because they will hydrogen bond to complementary 3′ or 5′ ends. As a result, the end of any DNA fragment produced by an enzyme, such as EcoRI, can anneal with any other fragment produced by that enzyme. This properly allows splicing of foreign genes into plasmids, for example. Some restriction endonucleases that may be particularly useful with the current invention include Bsu36I, HpaI, PspOMI, XbaI and XhoI.

Some endonucleases create fragments that have blunt ends, that is, that lack any protruding single strands. An alternative way to create blunt ends is to use a restriction enzyme that leaves overhangs, but to fill in the overhangs with a polymerase, such as Klenow, thereby resulting in blunt ends. When DNA has been cleaved with restriction enzymes that cut across both strands at the same position, blunt end ligation can be used to join the fragments directly together. The advantage of this technique is that any pair of ends may be joined together, irrespective of sequence.

Those nucleases that preferentially break off terminal nucleotides are referred to as exonucleases. For example, small deletions can be produced in any DNA molecule by treatment with an exonuclease which starts from each 3′ end of the DNA and chews away single strands in a 3′ to 5′ direction, creating a population of DNA molecules with single-stranded fragments at each end, some containing terminal nucleotides. Similarly, exonucleases that digest DNA from the 5′ end or enzymes that remove nucleotides from both strands have often been used. Some exonucleases which may be particularly useful in the present invention include Bal31, S 1, and ExoIII. These nucleolytic reactions can be controlled by varying the time of incubation, the temperature, and the enzyme concentration needed to make deletions. Phosphatases and kinases also may be used to control which fragments have ends which can be joined. Examples of useful phosphatases include shrimp alkaline phosphatase and calf intestinal alkaline phosphatase. An example of a useful kinase is T4 polynucleotide kinase.

Once the source DNA sequences and vector sequences have been cleaved and modified to generate appropriate ends, they are incubated together with enzymes capable of mediating the ligation of the two DNA molecules. Particularly useful enzymes for this purpose include T4 ligase, E. coli ligase, or other similar enzymes. The action of these enzymes results in the sealing of the linear DNA to produce a larger DNA molecule containing the desired fragment (see, for example, U.S. Pat. Nos. 4,237,224; 4,264,731; 4,273,875; 4,322,499 and 4,336,336, which are specifically incorporated herein by reference).

It is to be understood that the termini of the linearized plasmid and the termini of the DNA fragment being inserted must be complementary or blunt in order for the ligation reaction to be successful. Suitable complementary ends can be achieved by choosing appropriate restriction endonucleases (i.e., if the fragment is produced by the same restriction endonuclease or one that generates the same overhang as that used to linearize the plasmid, then the termini of both molecules will be complementary). As discussed previously, in one embodiment of the invention, at least two classes of the vectors used in the present invention are adapted to receive the foreign oligonucleotide fragments in only one orientation. After joining the DNA segment to the vector, the resulting hybrid DNA can then be selected from among the large population of clones or libraries.

Once a DNA vector has been prepared, it will be readily understood to those of skill in the art that infective RNA transcripts may be made therefrom. For example, commercial kits are available for production of RNA transcripts. On example of such a kit that was used by the inventors is the mMeSSAGE mMACHINE transcription kit from Ambion (Austin, Tex.).

In certain embodiments of the invention, techniques may thus be used to assay gene expression and generally, the efficacy of a given gene silencing construct. While this may be carried out by visual observation of a change in plant phenotype, molecular tools may also be used. For example, expression may be evaluated by specifically identifying the nucleic acid or protein products of genes. Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Very frequently, the expression of a gene product is determined by evaluating the phenotypic results of its expression. These assays also may take many forms including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may be observed, such as plant stature or growth.

### Characterization of Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on polypeptides encoded by the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use a bar-bialaphos or the EPSPS-glyphosate selective system, for example, transformed tissue can be cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate may be preferred, it is proposed that ranges of 0.1-50 mg/l bialaphos or 0.1-50 mM glyphosate will find utility.

### Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

It is understood that modifications which do not substantially affect the activity the various embodiments of this invention are also provided within the definition of the invention provided herein. Accordingly, the following examples are intended to illustrate but not limit the present invention.

### EXAMPLES

### Example 1: Construction of FoMV Based Silencing Vectors

The FoMV isolate 139 was first isolated from foxtail growing in a Kansas cornfield (Paulsen and Niblett, 1977). The isolate was kindly provided by Dr. Dallas Seifers (Kansas State University). The virus was maintained in sweet corn (Zea mays L. ‘Golden×Bantam’). Virus infected leaf sap was prepared by grinding infected leaves in 50 mM potassium phosphate buffer, pH 7.0. Sweet corn plants at the two-leaf stage were mechanically inoculated by rubbing leaf sap on new leaves dusted with 600-mesh Carborundum. To initiate infections from FoMV infectious clones, leaves of one week old plants were inoculated by particle bombardment using a Biolistic PDS-1000/He system (Bio-Rad Laboratories, Hercules, Calif., USA), 1.0 μm gold particles coated with 1 μg of FoMV plasmid DNA, and 1,100-psi rupture disks at a distance of 6 cm. Plants were placed in the dark for 12 h before and after bombardment and then maintained in a greenhouse room with a thermostat set to 20-22° C. with a 16 h photoperiod.

Unless otherwise stated, all plasmids were propagated in ElectroMax DH5a-E cells (Invitrogen, Carlsbad, Calif., USA) and purified using the QiaPrep Spin MiniPrep kit (Qiagen, Valencia, Calif., USA). All PCR was performed using Takara PrimeSTAR™ HS DNA Polymerase (TaKaRa Bio Inc., Otsu, Shiga, Japan). Nucleotide sequencing was done using the Big Dye Terminator DNA Sequencing Kit (Applied Biosystemns, Foster City, Calif., USA) and the ABI Prism 310 genetic analyzer at the Iowa State University DNA Facility. Sequence analysis was performed using the Vector NTI program (Invitrogen).

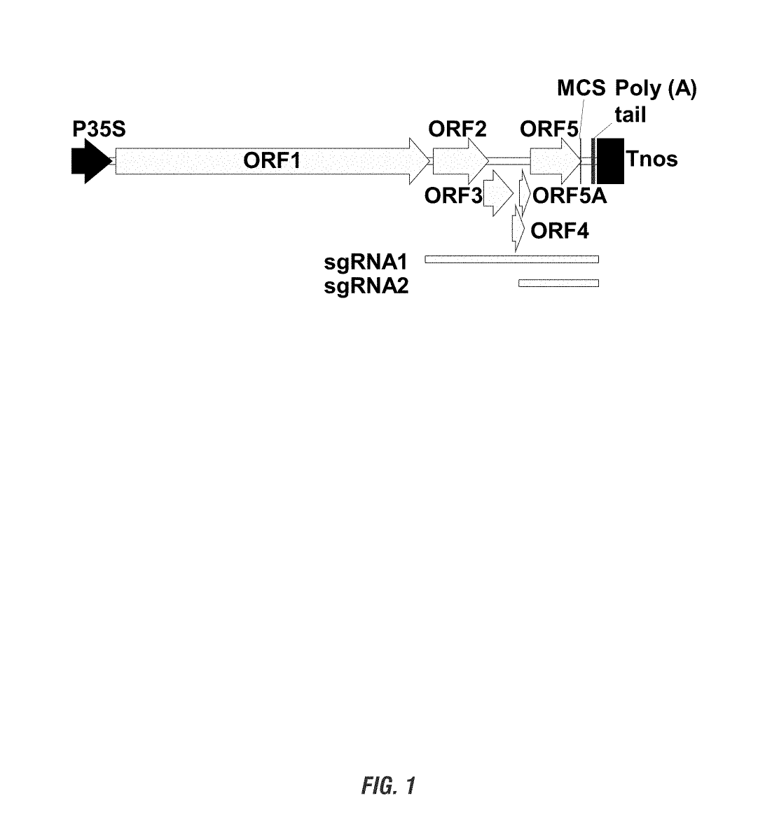

The full-length genomic cDNA of FoMV was obtained by two-step overlapping PCR and inserted into the SMV-NVEC plasmid at the StuI site. Specifically, total RNA extracted from FoMV-infected sweet corn leaves was used as a template for first-strand cDNA synthesis using 0.5 μg of mRNA, 0.5 μg oligo(dT)₂₀ primer, 1 μl 10 mM dNTP, and Superscript III reverse transcriptase (Invitrogen) to a final volume of 20 μl. The first-strand cDNA product (2 μl) was used as template in two 100 μl PCR reactions for amplification of the 5′ and 3′ends of the FoMV genomic cDNA using the primer pairs FM-5end & FM-2388R and FM-3end & FM-2388F, respectively. The PCR conditions were as follows: 1) 1 minute at 98° C. followed by three cycles of 98° C. for 10 seconds, 40° C. for 12 seconds, and 68° C. for six and one-half minutes; 2) 30 cycles 98° C. for 10 seconds, 52° C. for 12 seconds, and 68° C. for seven minutes; 3) 10 minutes at 68° C. The PCR products were gel extracted and used together as template in an overlap PCR reaction with primer pairs FM-5end & FM-3end for the generation of the FoMV full-length genomic cDNA. This PCR product was gel extracted, treated with T4 DNA kinase, and ligated into pSMV-NVEC that had been digested with StuI and dephosphorylated (Wang et al., 2006). This placed transcription of the FoMV genome under control of the Cauliflower mosaic virus 35S promoter and the nopaline synthase terminator (FIG. 1).

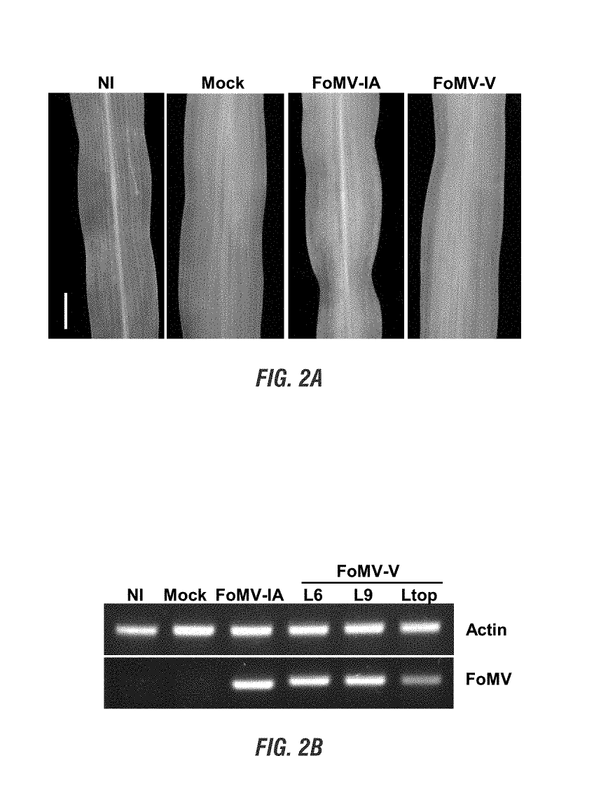

Clones in the correct orientation were screened by PCR with primer pair FM-5501F and NosRev. Genome orientation and fidelity of pFoMV-IA was further confirmed by sequencing with primer 35-Seq and with primers spanning the FoMV genome. Clones that had the correct insert size and orientation were biolistically inoculated onto sweet corn to test their infectivity. One clone, designated pFoMV-IA, reproducibly infected sweet corn, and so we obtained the complete genomic sequence. Analysis of the sequence of FoMV-IA cDNA showed that it has the expected genome organization based on previously published sequences (FIG. 1; Bruun-Rasmussen et al., 2008). FoMV-IA infected leaves display a mosaic pattern of light and dark green tissue that is indistinguishable from the symptoms produced by wild type FoMV infection on sweet corn (FIG. 2A). This is in contrast to the green leaves of healthy non-inoculated and mock-inoculated plants. To further confirm FoMV infection, RT-PCR analysis was performed using primers designed to amplify a 295 bp fragment from the FoMV genomic RNA. The PCR product was detected in symptomatic sweet corn plants that were biolistically inoculated with pFoMV-IA, but not in non-inoculated and mock-inoculated control plants (FIG. 2B).

To enable insertion of foreign sequences into the FoMV genome, the XbaI and XhoI restriction enzyme sites were inserted immediately after the stop codon of the capsid protein (CP, FIG. 1). In PCR reaction A, primer pair FM-5074F and FM-XbaRev was used to amplify a product from the wild-type FoMV-IA infectious clone, and the product was gel extracted. In PCR reaction B, primer pair FM-XbaFor and NosRev was used with wild-type infectious clone pFoMV-IA as a template, and the product was gel extracted. In overlap PCR reaction C, primer pair FM-5074F and NosRev was used with PCR products A and B as templates. PCR product C was digested with restriction enzymes SacII and ClaI, gel extracted, and ligated into pFoMV-IA that also had been digested with SacII and ClaI to produce the empty vector pFoMV-V. Inoculation of sweet corn with pFoMV-V showed that it was infectious and induced mosaic symptoms similar to inoculation with pFoMV-IA (FIG. 2A). The symptoms were first visible within about one week after biolistic inoculation, and continued to develop through 2 weeks after inoculation. Under our growth conditions, sweet corn plants appear to recover from infection, because symptoms are typically observed on only the 3^{rd} to 6^{th} leaves. However, viral fragments were detected by PCR throughout the infected plants indicating that FoMV continues to replicate and move systemically even though symptoms decrease (FIG. 2B).

### Example 2: Silencing of the Maize Phytoene Desaturase (Pds) Gene Using the FoMV Vector

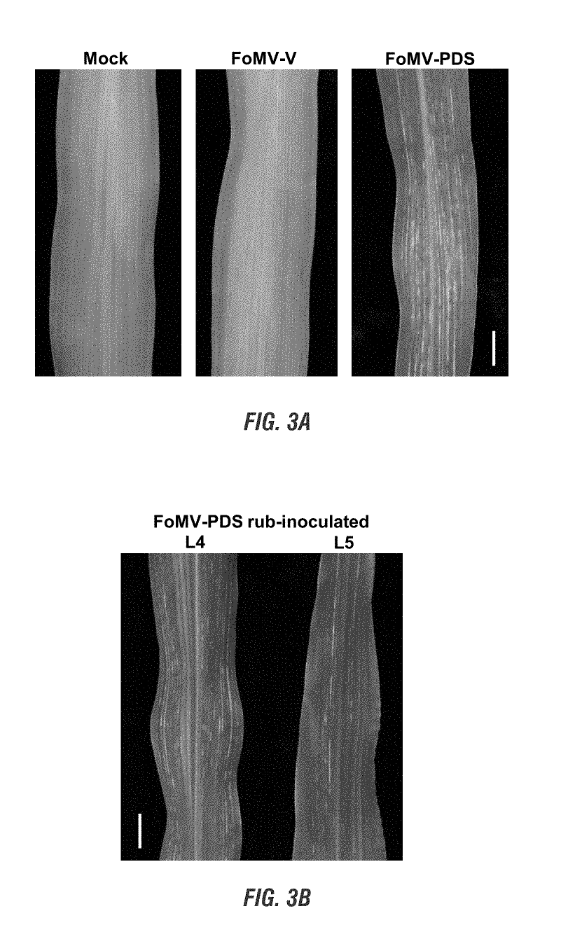

To test the ability of FoMV to induce silencing of an endogenous maize gene, we first tested pds, because it provides a striking visual marker when its expression is reduced sufficiently to cause a photo-bleaching phenotype (Holzberg et al., 2002; Ding et al., 2006). A 313-bp fragment corresponding to the 3′ end of the pds ORF was inserted in the anti-sense orientation at the XbaI and XhoI cloning site of pFoMV-V. RT-PCR analysis was performed to detect the presence of insert using primer pair FM-5840F and FM-6138R. Sweet corn seedlings at the two-leaf stage were inoculated with pFoMV-PDS construct by biolistic bombardment. Photo-bleaching was first observed at about ten days after inoculation with pFoMV-PDS, and this phenotype became more obvious at two weeks after inoculation (FIG. 3). A mosaic pattern of light and dark green tissue was observed on plants inoculated with the pFoMV-V empty vector control, and these symptoms were distinct from the photo-bleaching phenotype caused by FoMV-PDS (FIG. 3A). The phenotypes caused by FoMV-PDS were observed from the 3^{rd} to the 10^{th} leaf, with the majority of plants displaying photo-bleaching from the 4^{th} to the 9^{th} leaf.

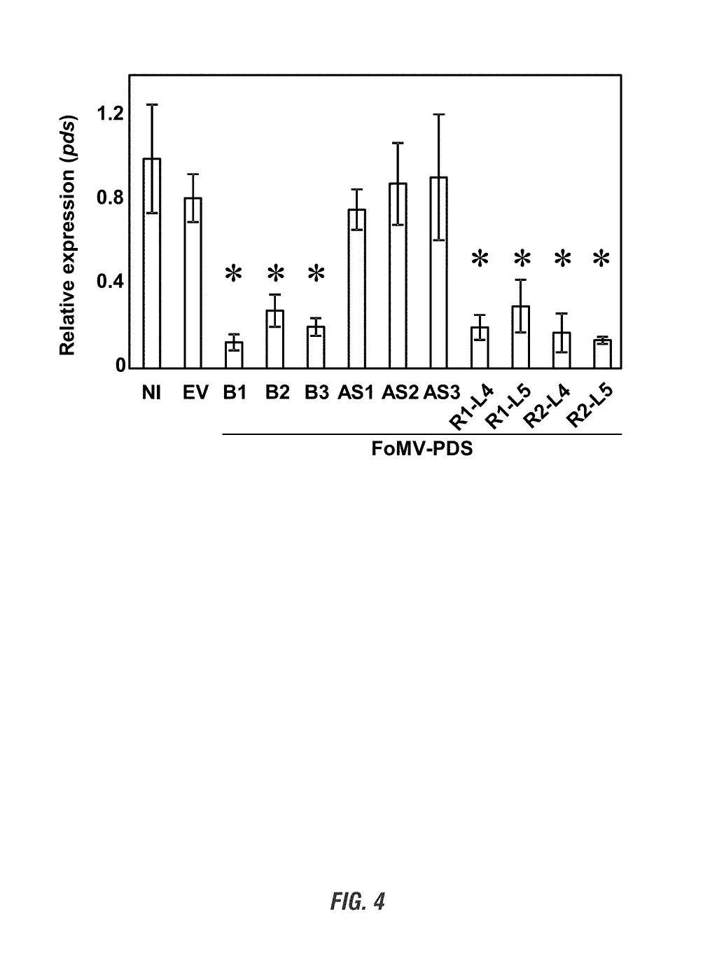

To evaluate the effectiveness of VIGS of the target gene, pds mRNA transcript levels were compared among control plants (mock treated or infected with the empty FoMV-V) and FoMV-PDS-infected sweet corn leaves with photo-bleaching phenotypes. Total RNA was extracted from the 4^{th} leaves of these plants, and the accumulation of pds mRNA transcripts was quantified using qRT-PCR. Non-infected wild type leaves, pFoMV-V or pFoMV-PDS infected leaves were harvested for total RNA extraction using RNeasy Plant Mini Kit (Qiagen, Valencia, Calif.). Individual leaves as indicated in figures were harvested from each plant. After first-strand cDNA synthesis, primer pair PDSrt3 and PDSrt4 was used to detect the expression of Pds mRNA by qRT-PCR using iQ™ SYBR® Green Supermix on an iCycler real-time PCR detection system (Bio-Rad, Hercules, Calif.). Actin was used as an internal control with primer pair ZmActrtS and ZmActrtR. The expression level of pds in non-infected leaves was set as 1.0 and the delta-delta Ct method was used for comparing the expression levels of pds in pFoMV-V and pFoMV-PDS infected samples. The expression level of pds was similar between FoMV-V infected and non-inoculated sweet corn leaves demonstrating that FoMV infection alone did not affect pds mRNA expression (FIG. 4). In contrast, FoMV-PDS infection resulted in a significant reduction of pds expression in photo-bleached leaves, with levels ranging from 13.5%-27.6% of that of the non-silenced controls. This result showed that RNA silencing induced by FoMV-PDS can reduce expression of the target gene by 3.6 to 7.4 fold compared with the non-silenced empty vector control. In addition, pds transcript levels were quantified using qRT-PCR in leaves of plants that had been inoculated with pFoMV-PDS but did not display the photo-bleaching phenotype. We observed that pds mRNA levels were not significantly reduced (FIG. 4). These results show that asymptomatic leaves from plants infected with the silencing construct contain similar expression levels to that of the mock and empty vector control, and they further confirm that reduced levels of pds mRNA transcripts are correlated with the photo-bleaching phenotype.

To test whether the silencing effect of FoMV-PDS could be passaged, the sap from leaves of the biolistically-inoculated plants displaying obvious photo-bleaching was used to rub-inoculate naïve sweet corn seedlings. Photo-bleaching occurred after eight to nine days on the rub-inoculated plants demonstrating that the ability to induce the pds silencing phenotype could be passaged (FIG. 3B). Similar to procedures described above, pds mRNA expression levels were evaluated for leaves four and five (L4 and L5) on rub-inoculated plants displaying photo-bleaching on two independent rub-inoculated plants. We observed that the expression of pds was significantly reduced in the photo-bleached leaves of the passaged plants (FIG. 4). Taken together, these data showed that pds was silenced to a similar extent when leaf sap from pFoMV-PDS-inoculated plants was rub-inoculated onto new sweet corn plants.

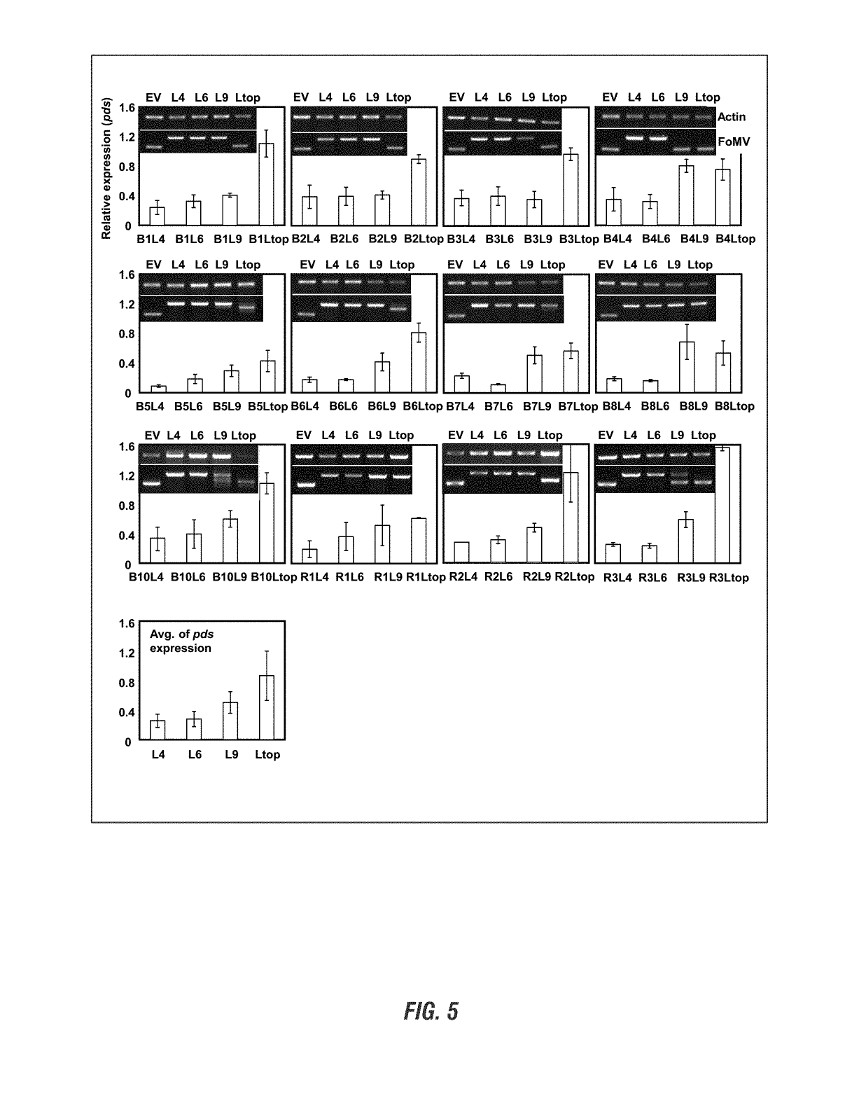

We observed that most of the FoMV-PDS-infected plants had photo-bleaching on the 4^{th} to 9^{th} leaves. However, the phenotype was not uniform across all leaves and could be described as a gradient from bottom to top. The most clear and obvious photo-bleaching was observed on the 5^{th} and 6^{th} leaves, and then the phenotype became less severe in the upper leaves. We reasoned that this lack of uniform phenotype could be due to instability of the 313 bp pds fragment. To investigate the stability of the pds fragment in FoMV-PDS and its relationship to pds silencing throughout maize plants, we conducted RT-PCR analyses on RNA extracted from the following leaves: 4, 6, 9 and the top most leaf (usually 13, but occasionally 12 or 14). These leaves are designated as L4, L6, L9 and L top. The RT-PCR assays used FoMV primers that flanked the cloning site to detect intact FoMV-PDS or deletion derivatives, and qRT-PCR was used to quantify pds mRNA accumulation in the same samples (FIG. 5, graphs show relative pds mRNA expression and the insets show corresponding FoMV-PDS PCR products). A total of 12 FoMV-PDS-infected plants were examined, including nine inoculated by bombardment (passage 0) and three that were rub-inoculated (passage 1). Deletion of the pds insert was not detected in any L4 or L6 sample. However, four of 12 L9 samples contained deletions of the pds fragment to different extents (one minor, two partial, and one complete), and 11 of the L top samples had deletions of the pds fragment (two minor, four partial, and five complete). The deletions were more extensive in the L top samples, with six of them showing PCR products consistent with complete deletion. These results corresponded well with the level of pds mRNA transcripts detected by qRT-PCR, with less silencing observed as the frequency and extent of deletions increased. FoMV-PDS caused pds mRNA transcripts to be reduced to 25.4% and 27.8% of their levels in L4 and L6 empty vector control samples (FIG. 5). In L9, FoMV-PDS silenced pds expression by half, while no significant pds silencing was detected in L top samples. Furthermore, when comparing rub-inoculated and biolistically-inoculated plants, no obvious difference was observed (FIG. 5).

We have demonstrated that when the FoMV vector was used to silence pds obvious leaf photo-bleaching was observed that was consistent with pds silencing in other plant species. The mRNA transcript levels of pds were reduced by approximately 70%-80% in L4 and L6 samples in both biolistically inoculated plants and subsequent rub-inoculated plants, indicating a high efficiency of this FoMV VIGS system in certain leaves. Stable and sustainable gene silencing was also obtained using mechanical inoculation passage through leaf sap prepared from L5 of FoMV-PDS biolistically inoculated plants. In upper leaves (L9 and L top), the silencing effect was reduced, which was consistent with the deletion of the pds fragment.

### Example 3: Silencing of the Maize Lesion Mimic 22 (les22) Using the FoMV Vector

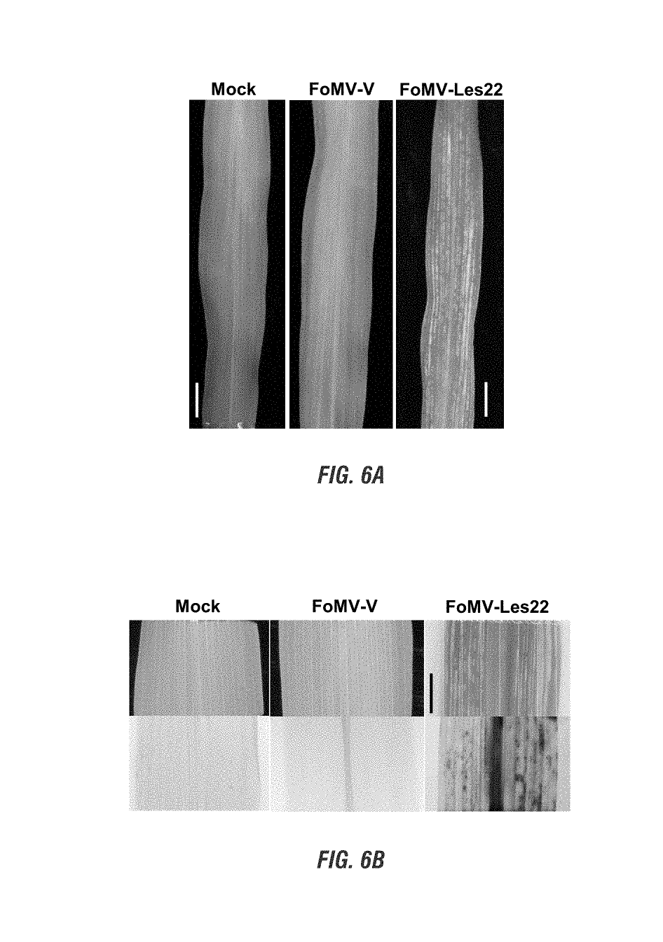

To further test the ability of FoMV to silence maize genes, we targeted the lesion mimic gene, les22. A 329 bp fragment corresponding to the 3′ end of the les22 ORF was inserted in a reverse orientation at the XbaI and XhoI cloning sites in pFoMV-V. Sweet corn plants were inoculated with pFoMV-Les22 using biolistic bombardment. Null mutations in les22 result in the appearance of necrotic spots on leaves that resemble the cell death triggered during a hypersensitive response to plant pathogens (Hu et al., 1998). Characteristic necrotic spots began to appear at 8-10 days after inoculation with FoMV-Les22. This lesion mimic phenotype became more obvious by two weeks after inoculation and spread to all the leaves later on (FIG. 6A). When symptomatic leaf sections were stained with trypan blue, a histochemical assay for irreversible membrane damage indicative of cell death, blue spots were observed in brownish areas on the leaf confirming the occurrence of cell death (FIG. 6B). These results indicate that FoMV-Les22 infection is able to induce a les22 silencing phenotype similar to that of les22 null mutants. Trypan blue staining was performed as follows: Leaf sections were submerged trypan blue solution (25% trypan blue stock solution (0.4%), 25% lactic acid, 25% water-saturated phenol, 25% glycerol) at 70° C. and infiltrated for 1 min, then placed in boiling water for 2 min, and stained overnight at room temperature. Samples were de-stained in a chloral hydrate solution (25 g in 10 ml of H₂O) followed by equilibration in 70% glycerol for observation.

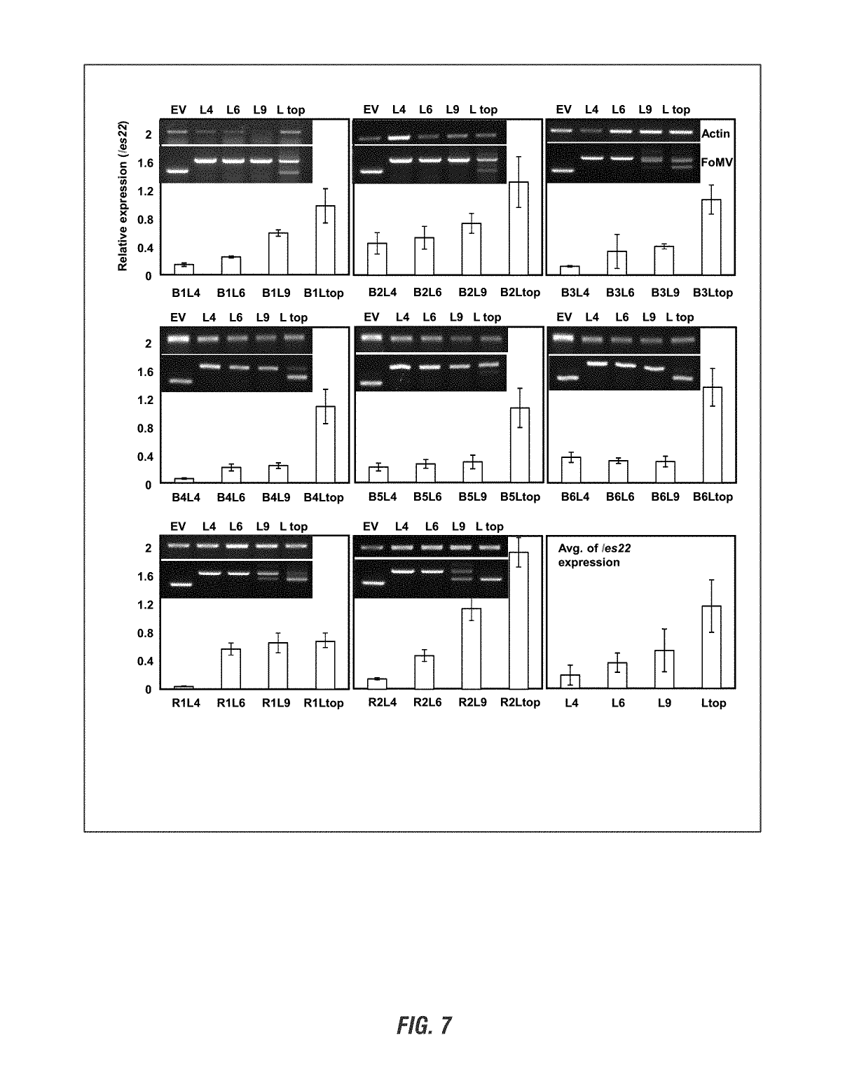

The lesion mimic phenotype was first observed on the 3^{rd} leaf of FoMV-Les22 infected plants. As plants grew, the phenotype spread over all the leaves, although only tiny necrotic spots were observed on the top leaves. In most plants, severe cell death was observed on leaves 4 to 6. The lesion mimic phenotype became less severe as the virus spread to upper leaves, forming a phenotypic gradient from bottom to top similar to FoMV-PDS. We tested the stability of the 329 bp Les22 insert in L4, L6, L9, and L top samples from eight FoMV-Les22 plants. No deletion was detected in any L4 and L6 samples, partial deletions were detected in three L9 samples, and partial or total deletions were detected in all L top samples. These results indicate that the insert was gradually lost as FoMV-Les22 moved into the upper leaves. There was no obvious difference in insert stability or les22 silencing effect between bombardment-inoculated (passage 0) and rub-inoculated (passage 1) plants. Quantification of les22 mRNA levels with primer pair Les22rtS and Les22rtA showed that its expression was suppressed by 81.9% in L4, 63.1% in L6, and 45.4% in L9 and no suppression was detected in L top samples (FIG. 7).

When les22 was targeted for silencing, necrotic spots were observed on leaves, which is a phenotype that is consistent with les22 null mutations. The silencing effect of les22 that we observed was similar to pds, indicating that the silencing effects caused by FoMV-PDS and FoMV-Les22 are typical for this version of the FoMV system.

### Example 4: Silencing iojap and Brown midrib3 Using the FoMV Vector

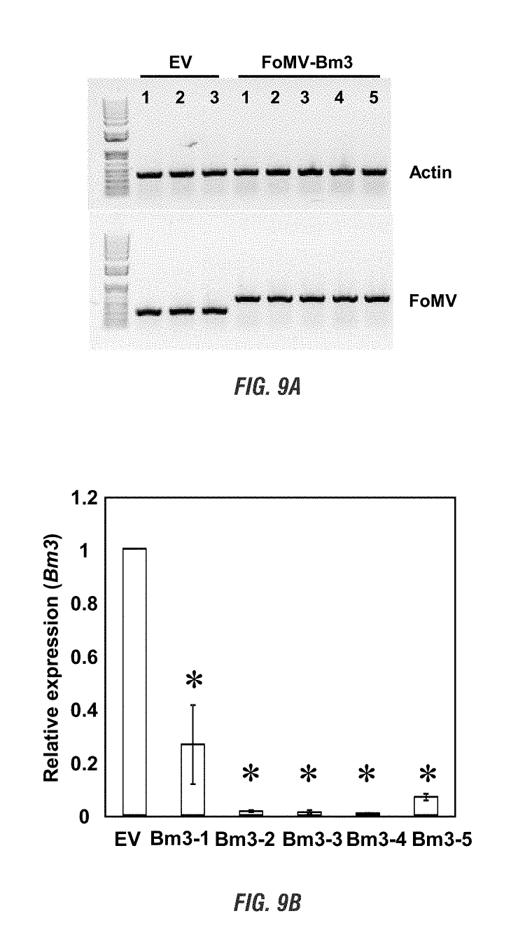

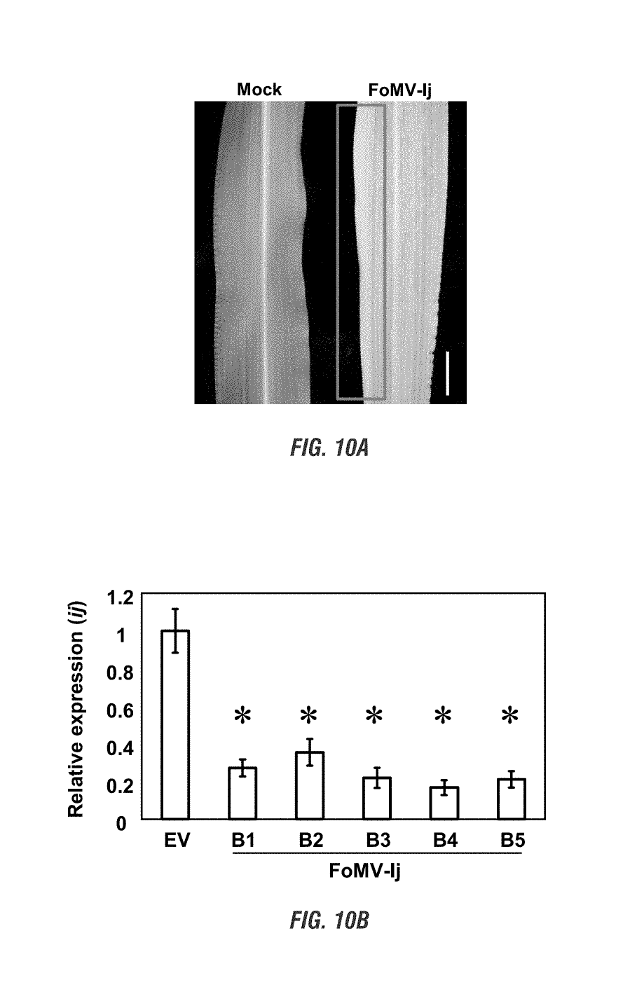

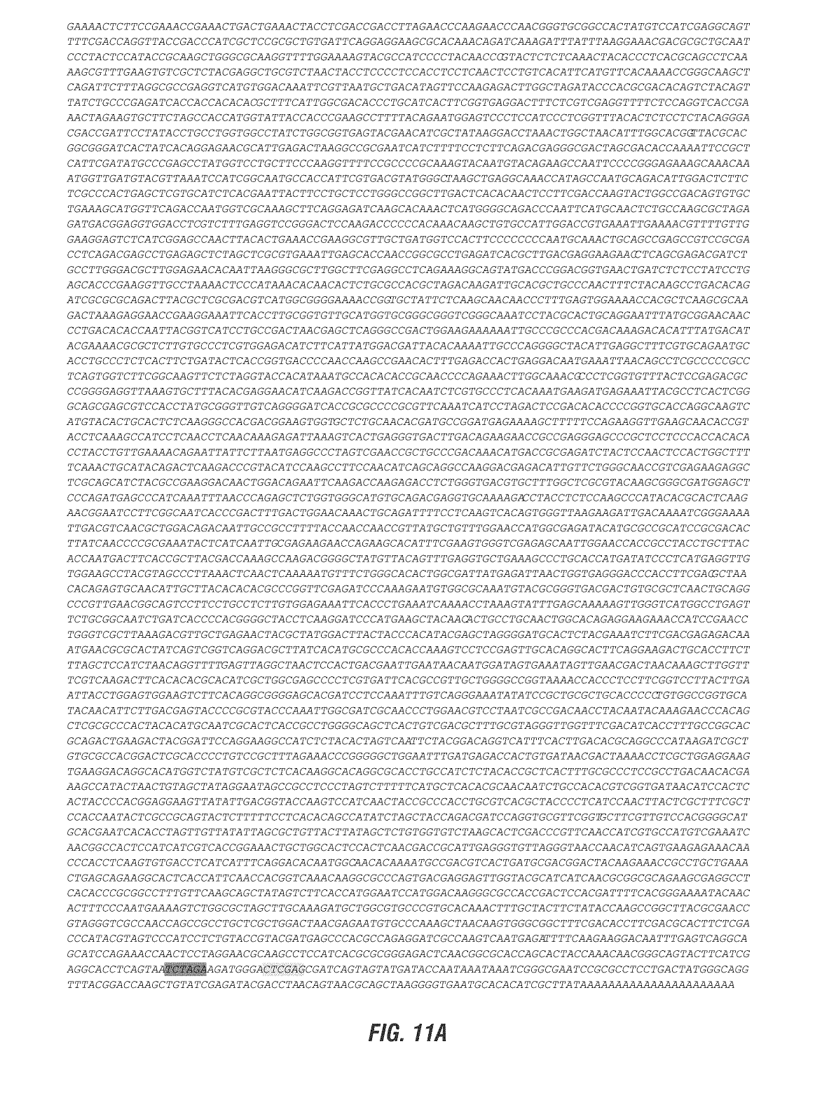

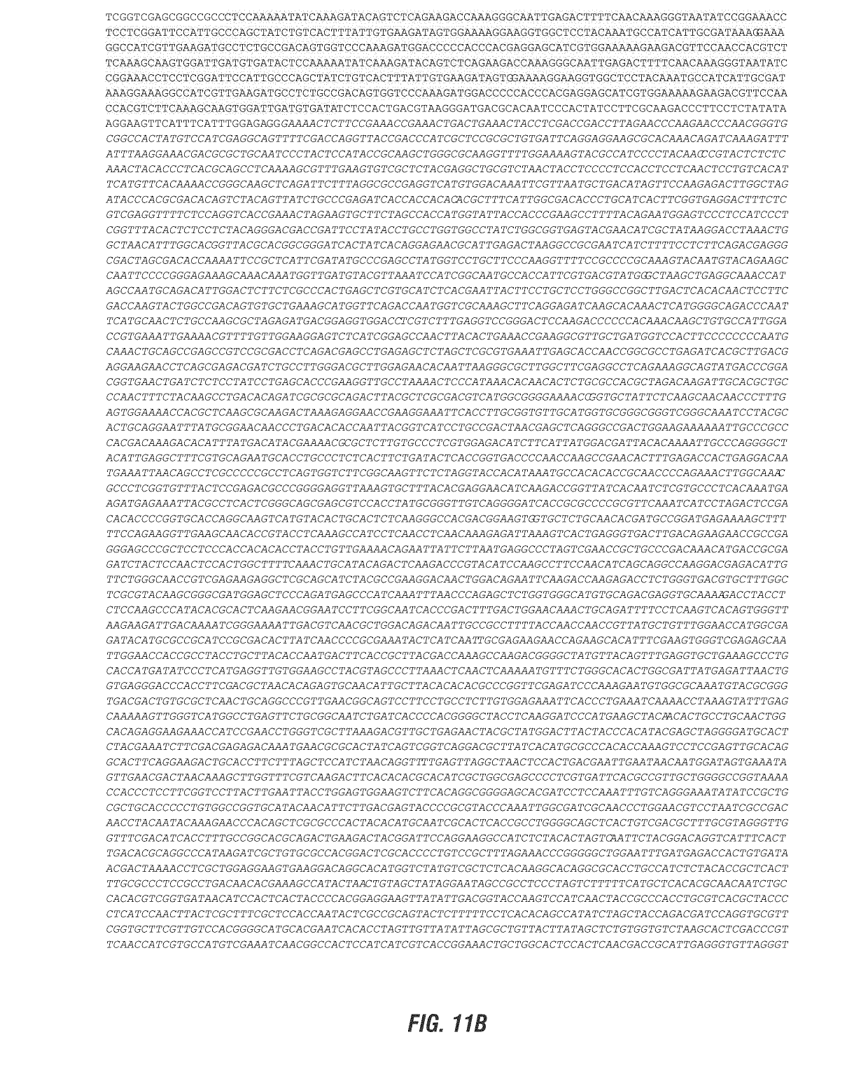

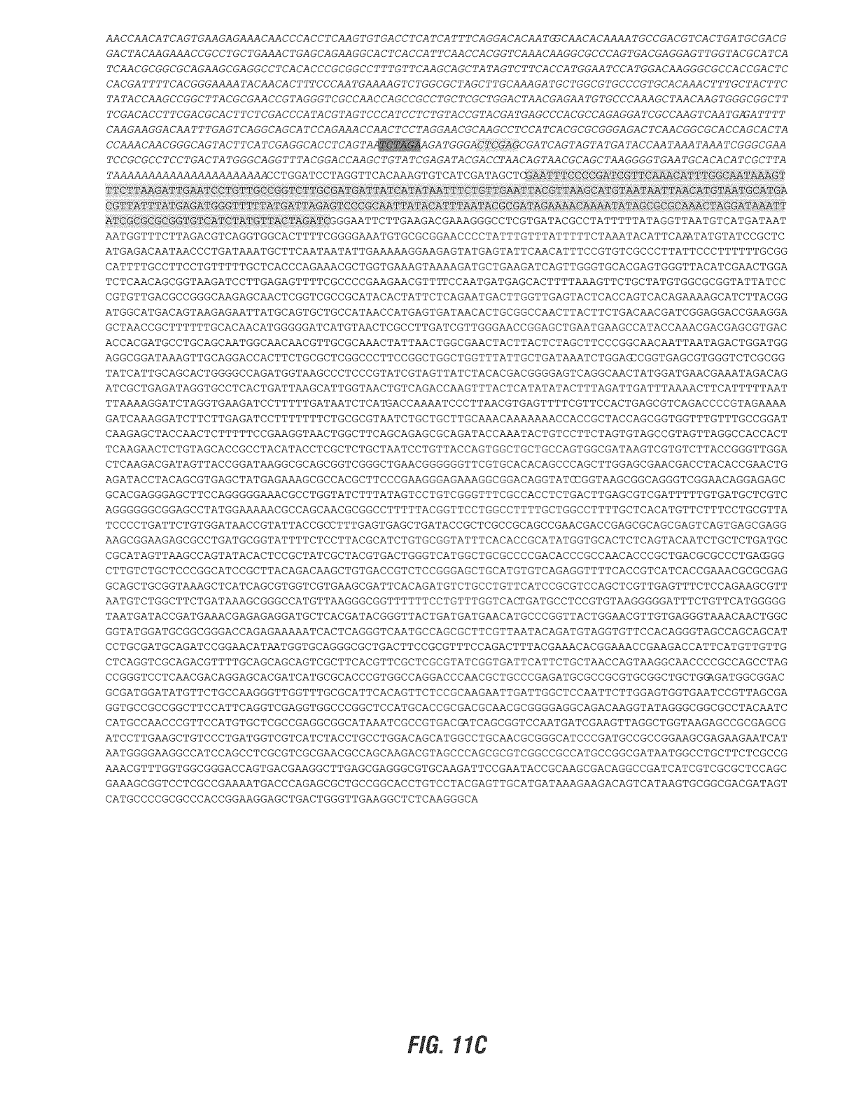

In addition to pds and les22, we targeted two other genes using the FoMV vector that were chosen because they were expected to produce different types of loss-of-function phenotypes in leaves. These genes were iojap (ij, Han et al., 1992) and brown midrib 3(bm3, Vignols et al., 1995). We inserted cDNA fragments corresponding to the last approximately 300 bp of the open reading frames of these genes between the XbaI and XhoI sites in pFoMV-V in a reverse orientation. The resulting constructs were named pFoMV-Ij and pFoMV-Bm3. These constructs were biolistically inoculated onto sweet corn. For FoMV-Bm3 (FIG. 9), viral symptoms were observed that were similar to pFoMV-V infected control plants. However, no reddish brown pigmentation was observed in the leaf midribs, which is expected for bm3 loss-of-function mutants (Barrière and Argillier, 1993). RT-PCR analysis confirmed that the Bm3 fragment was present in the viral genome of systemically infected plants, and qRT-PCR analysis with primer pair Bm3rtS and Bm3rtA demonstrated that expression of the Bm3 mRNA was significantly decreased by 73.5%-99.3%. These data show that Bm3 was silenced even though the brown midrib phenotype was not observed. For FoMV-Ij (FIG. 10), white stripes were observed on leaves of infected plants, forming typical white margin patterns. Real-time RT-PCR analysis was performed to test the expression level of if on the 6^{th} leaf of infected plants on which severe phenotype was observed. The results showed that in five independent plants, ij expression level is only 16.7%-35.5% of that in empty vector-infected controls, indicating significant suppression effect.

### Example 5: FoMV Infection of Maize Inbreds, Sorghum, and Green Foxtail

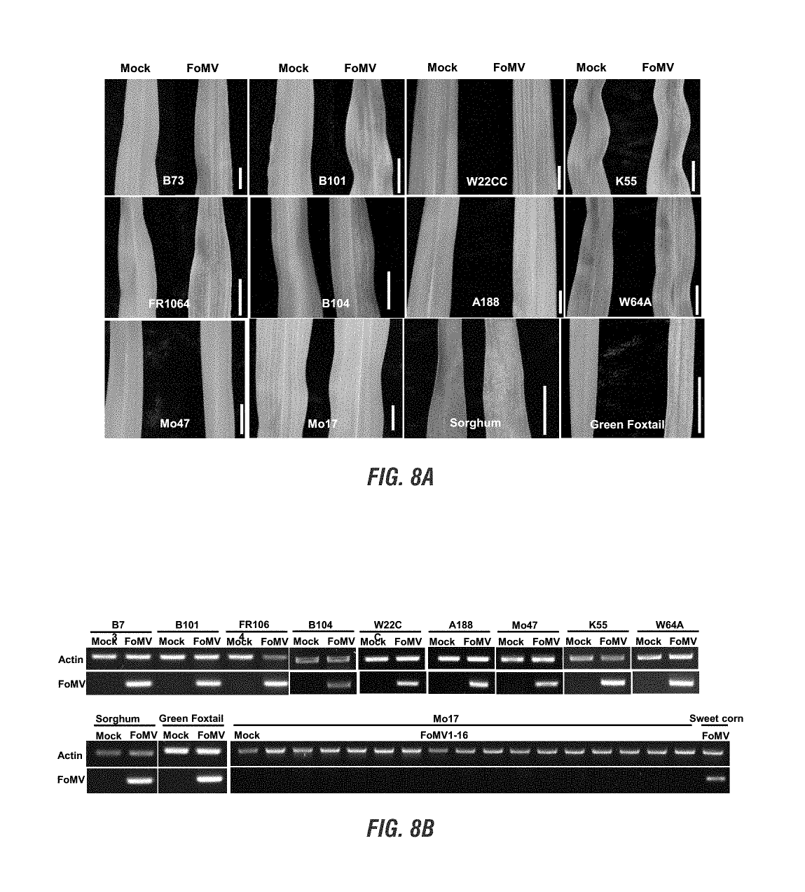

We were interested to know if our FoMV infectious clone might have utility in other maize genotypes and other grass species. To test this, seedlings of sorghum, green foxtail, and different maize inbred lines were rub-inoculated with the FoMV-V virus (FIG. 8). Mosaic symptoms were observed on leaves of maize inbred lines B73, B101, W22CC, K55, FR1064, B104, A188, and W64A. No viral symptoms were observed in inbred lines Mo17, Mo47. RT-PCR further confirmed FoMV infection in symptomatic leaves and also in non-symptomatic leaves from Mo47 that are inoculated with FoMV-V. No infection was detected in 16 individual Mo17 plants that were inoculated with FoMV-V, indicating a resistance of Mo17 on FoMV, which is consistent with a previous publication (Ji et al., 2010). FoMV-V was infectious in sorghum (Sorghum bicolor) and green foxtail (Setaria viridis) plants as evidenced by the mosaic symptoms that developed on leaves and the presence of a band in RT-PCR These data indicate that it will be feasible to use the FoMV vector to silence genes in other maize genotypes and plant species of economic and scientific interest.

### Example 6: Construction of FoMV Based Expression Vectors

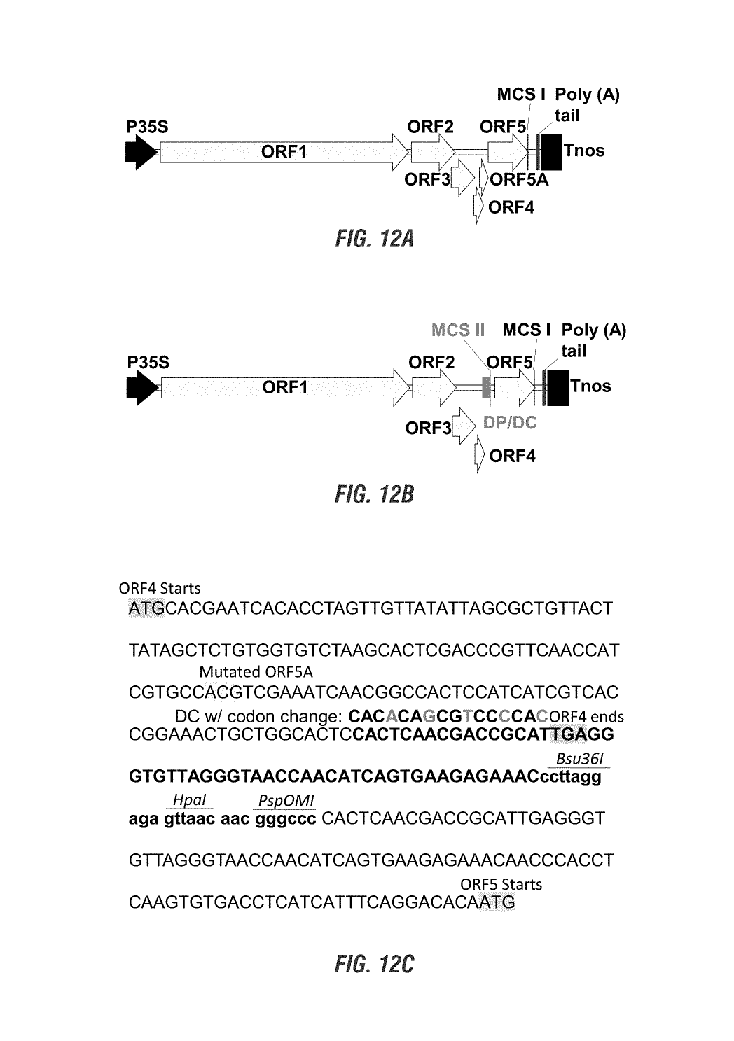

The pFoMV-V vector for VIGS cannot be used to express proteins, because the foreign inserts are placed after the stop codon of ORF5 (FIG. 12A). To engineer the FoMV vector for gene expression, the promoter duplication strategy similar to what has been used in PVX vectors was employed (Sablowski et al., 1995; Lacomme and Chapman, 2008; Dickmeis et al., 2014; Wang et al., 2014).

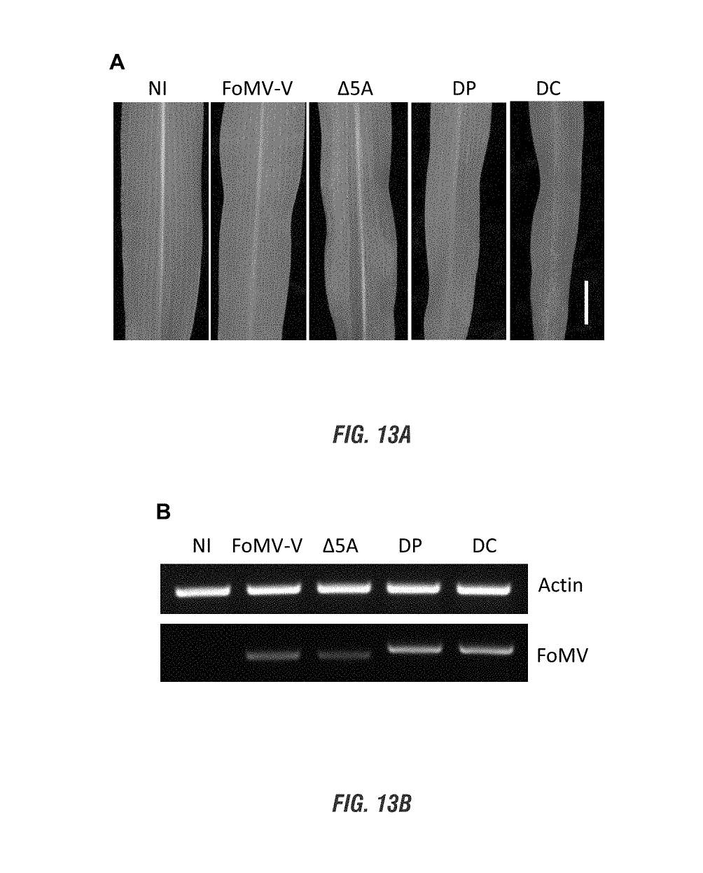

In order to make a modified pFoMV-V that lacks ORF5A, a mutation in pFoMV-V which changes the start codon of ORF5A from ATG to ACG without changing the amino acid derived from ORF4 was introduced (FIG. 12C). In PCR reaction A, primers 5AmuS1 and 5AmuA1 were used to amplify a product from pFoMV-V and the product was gel extracted. In PCR reaction B, primers 5AmuS2 and 5AmuA2 were used with pFoMV-V as template and the product was gel extracted. In overlap PCR reaction C, primers 5AmuS1 and 5AmuA2 were used with PCR products A and B as templates. PCR product C was digested with restriction enzymes SacII and SalI, gel extracted, and ligated into pFoMV-V that also had been digested with SacII and SalI to produce the pFoMV-V-Δ5A vector. The infectivity of pFoMV-V-Δ5A was tested by both biolistic inoculation and rub inoculation, and no significant difference was observed compared to pFoMV-V. Plants infected by pFoMV-V-Δ5A show a mosaic pattern of light and dark green tissue similar to those infected by pFoMV-V (FIG. 13A). The stability of the mutation was tested in 14 biolistically inoculated plants and 13 rub inoculated plants, and the mutation was detected in all the infected samples. These results indicate that deletion of 5A does not impair the infectivity of the mutated FoMV and the mutation is stable. Therefore, pFoMV-V-Δ5A is used for further modification.

To add a multiple cloning site in pFoMV-V-Δ5A, primers 5AmuS1 and 201DPA1 were used to amplify a product from pFoMV-V-Δ5A in PCR reaction D and the product was gel extracted. In PCR reaction E, primers 201DPS1 and 5AmuA2 were used with pFoMV-V-Δ5A as template and the product was gel extracted. In overlap PCR reaction F, primers 5AmuS1 and 5AmuA2 were used with PCR products D and E as templates. PCR product F was digested with restriction enzymes SacII and SalI, gel extracted, and ligated into pFoMV-V that also had been digested with SacII and SalI to produce the pFoMV-V-Δ5A-MSC, which contains PmlI, Bsu36I, HpaI and PspOMI. To insert the putative subgenomic promoter for CP into pFoMV-V-Δ5A-MCS, oligonucleotides DPS and DPA were synthesized and annealed to form double stand DNA fragment that contains the putative subgenomic promoter for CP (5280-5333) and Bsu36I recognition site at 3′ end. The annealed product was digested with Bsu36I and ligated into pFoMV-V-Δ5A-MCS that had been digested with PmiI and Bsu36I to produce the pFoMV-DP.

The resultant vector pFoMV-DP contains a duplication of the putative FoMV subgenomic promoter for CP (54-bp, 5280-5333) followed by the MCSII which contains Bsu36I, HpaI and PspOMI (FIG. 12B,C). In order to minimize potential homologous recombination, which would cause instability, a modified version, pFoMV-DC was made by changing codons in the area that overlaps with ORF4 in a way that the derived amino acid is not altered (FIG. 12C). The pFoMV-DC was constructed similarly using synthesized oligonucleotides DCS and DCA.

Inoculation of sweet corn with pFoMV-DP or pFoMV-DC showed that both constructs were infectious and induced mild mosaic symptoms that were indistinguishable from those of plants inoculated with the parental pFoMV-V or pFoMV-V-Δ5A clones (FIG. 13A). To further confirm FoMV infection, RT-PCR analysis was performed using primers designed to amplify a 396 bp fragment from the FoMV genomic RNA. The PCR product was detected in symptomatic sweet corn plants that were biolistically inoculated with pFoMV-V, pFoMV-V-Δ5A, pFoMV-DP, or pFoMV-DC, but not in non-inoculated control plants. A slightly larger band was detected in pFoMV-DP or pFoMV-DC infected tissues, indicating the presence of the duplicated promoter (FIG. 13B).

### Example 7: Expression of Bialaphos Herbicide Resistance (BAR) Gene Using the FoMV Vector

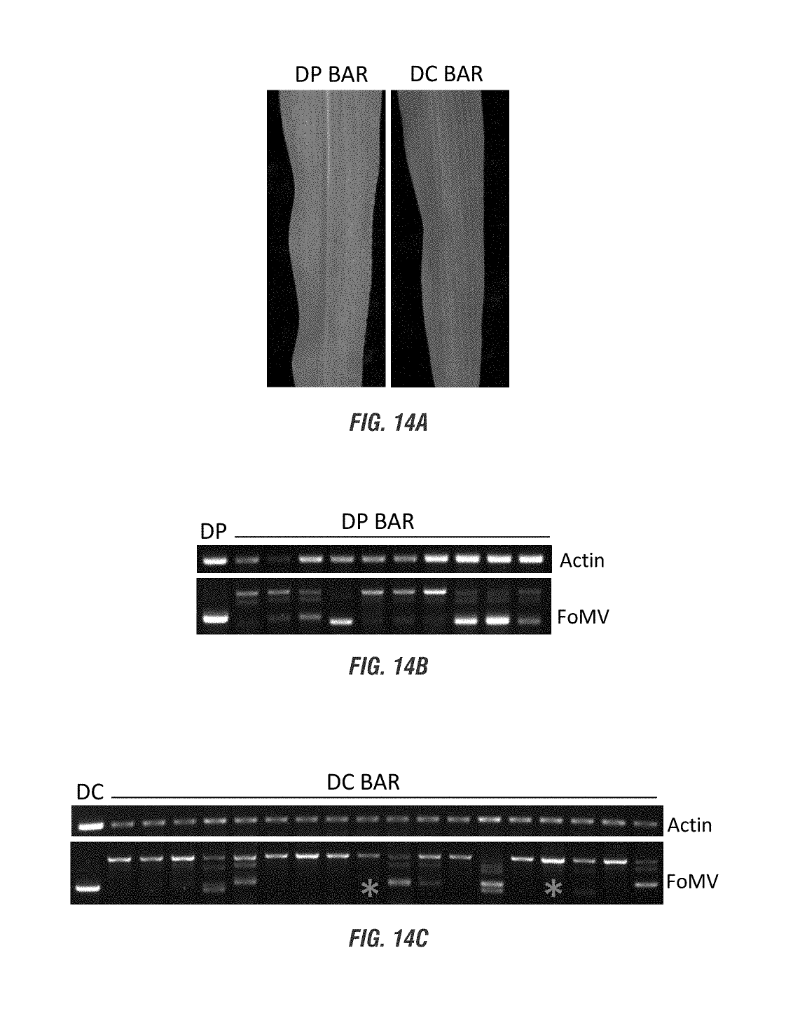

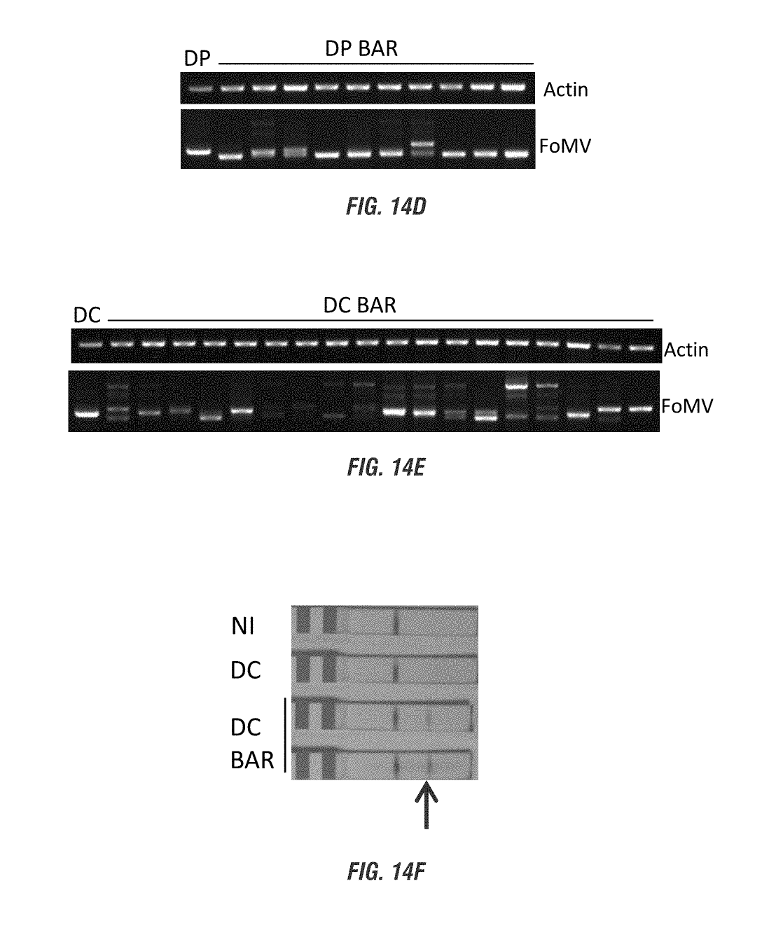

To test the potential for foreign gene expression, we first tested bialaphos herbicide resistance (BAR) gene which encodes a product that can protect plants from Finale® (Agrevo) herbicide. The BAR coding region was amplified by PCR using plasmid pBPMV-GFP-BAR (Zhang et al., 2010) as template and primer pair BAR Bsu36I and BAR HpaI. The product was cloned into pGEM-T easy vector and sequenced for verification. The BAR encoding fragment was released by Bsu36I and PspOMI double digestion and ligated into similarly digested pFoMV-DP and pFoMV-DC to produce pFoMV-DP-BAR and pFoMV-DC-BAR, respectively. About ten-days after biolistic inoculation, 50-90% of inoculated plants became systemically infected. Typical mosaic symptoms were observed in both pFoMV-DP-BAR and pFoMV-DC-BAR infected plants (FIG. 14A) with most of them starting to show viral symptom from the 3^{rd} leaf. To further confirm infection and also to test stability of the insertion, leaf samples from the 4^{th} and 6^{th} leaves of 10 pFoMV-DP-BAR infected plants and 18 pFoMV-DC-BAR infected plants were collected for RT-RCR analysis using FoMV primers that flanked the cloning site to detect intact FoMV-DP/DC-BAR or deletion derivatives. As shown in FIG. 14B,C, while a 475-bp PCR product was detected in the tissue infected by the empty vector, in the pFoMV-DC/DP-BAR infected tissue, a 1027-bp PCR product was detected instead, indicating the presence of intact BAR insertion. When tested in the 4^{th} leaf samples, this PCR product was detected in 17 out of 18 pFoMV-DC-BAR infected plants, and 6 of them also showed significant deletion derivatives. In the 4^{th} leaf samples from pFoMV-DP-BAR infected plants, the 1027-bp PCR product was detected in 9 out of 10 plants, and all of them also showed significant deletion derivatives. When the same analysis was performed in the 6^{th} leaf samples, the deletion of BAR insertion became more extensive, where all the pFoMV-DP/DC-BAR infected plants showed partial to total deletions (FIG. 14D,E). Overall, BAR insertion is less stable in pFoMV-DP vector than in pFoMV-DC vector, which is expected since the pFoMV-DC vector is designed to minimize potential recombination. In addition to the detection on the RNA level, we also detected the expression of BAR protein using the EnviroLogix QuickStix Kit for LibertyLink® (bar). The presence of BAR protein was detected only in pFoMV-DC-BAR infected plants but not in non-infected plant or plant infected by pFoMV-DC control. (FIG. 14F).

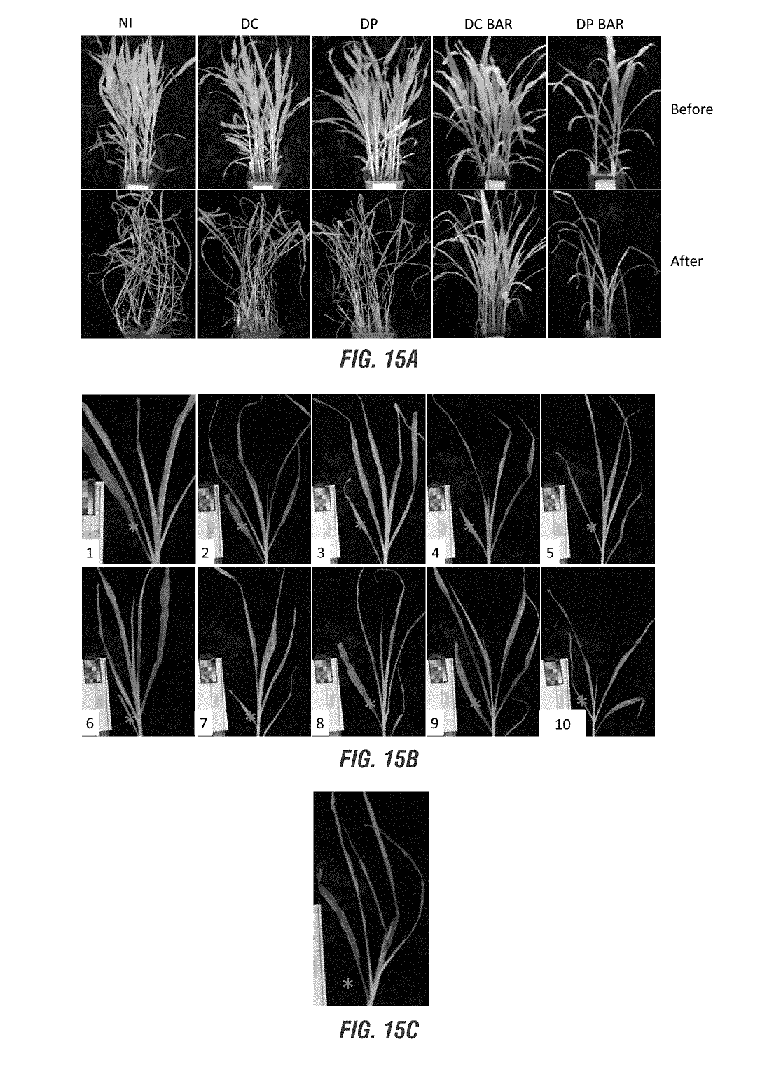

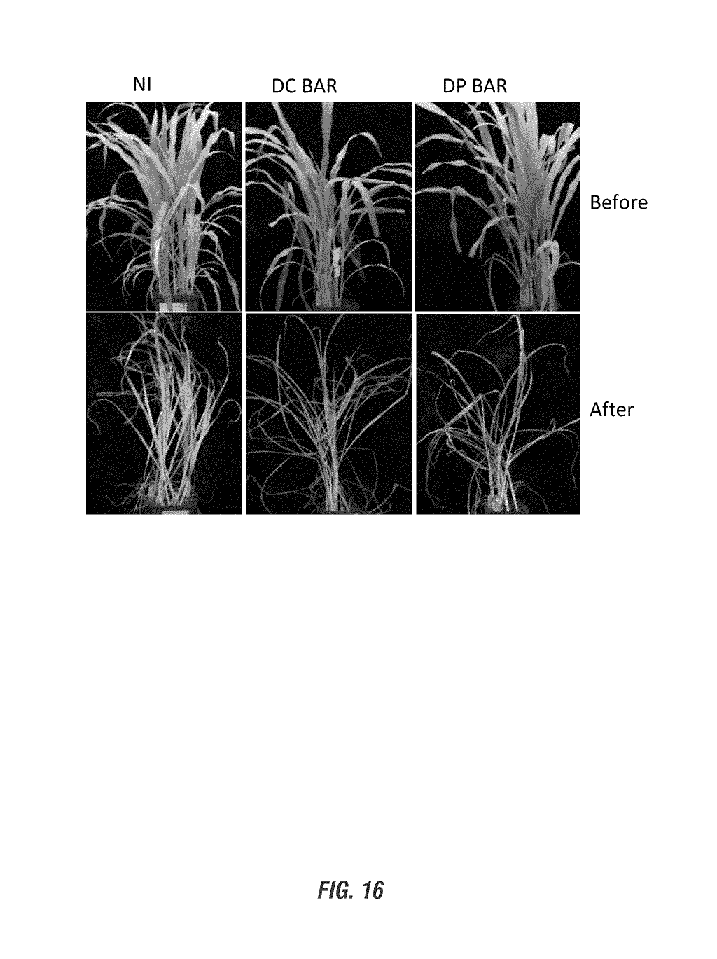

To further test the function of FoMV-mediated BAR expression, plants were challenged by Finale® (Agrevo) herbicide which contains glufosinate-ammonium as the active ingredient (Aventis Crop-Science). Plants were sprayed with 0.05% Finale solution (w/v) in deionized water twice at a 3-day interval and pictures were taken ten days after treatment. As shown in FIG. 15, at 18 DPI before herbicide treatment, wild-type non-infected plants, pFoMV-DC/DP infected plants and pFoMV-DC/DP-BAR infected plants were all green and healthy (besides leaf mosaic in FoMV infected plants). After herbicide treatment, wild-type non-infected plants, pFoMV-DC/DP infected plants were killed while plants infected by pFoMV-DC/DP-BAR were partially or totally protected. The protective effect varies among plants, with most having at least the 4^{th} leaf remaining green. However, when herbicide was treated at a later stage, even the pFoMV-DC/DP-BAR infected plants were killed (FIG. 16), which is possibly because of more extensive deletion of the BAR gene.

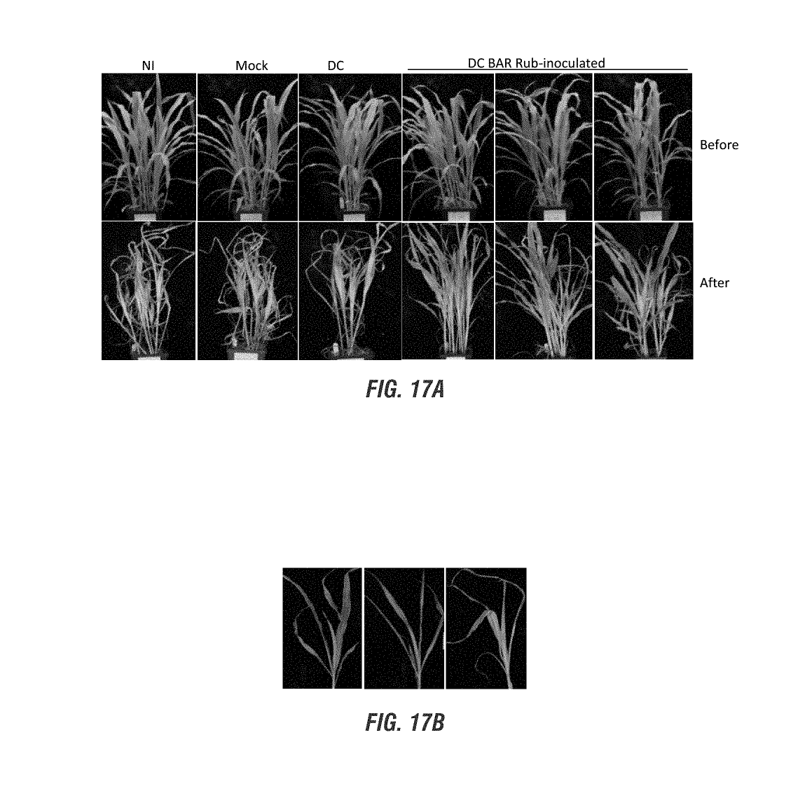

To test whether the expression of BAR could be passaged, the sap from leaves of the biolistically-inoculated plants with confirmed BAR expression was used to rub-inoculate the first two leaves of naïve sweet corn seedlings. Mosaic leaf symptom was observed about one week after rub-inoculation in 25 out of 30 plants. Similar to procedures described above, these plants were challenged by Finale® (Agrevo) herbicide at 13 DPI (together with wild-type and mock-treated non-infected controls and also with plants infected by the empty vector). We observed that 16 FoMV-DC-BAR infected plants were protected from herbicide treatment with at least one leaf remaining green and 8 of them showed all green leaves 10 days after treatment (FIG. 17). These data showed that functional BAR protein is expressed when leaf sap from pFoMV-DC-BAR-inoculated plants was rub-inoculated onto new sweet corn plants, and the protective effect is similar in biolistic and rub-inoculated plants.

### Example 8: Expression of Green Fluorescent Protein (GFP) Gene Using the FoMV Vector

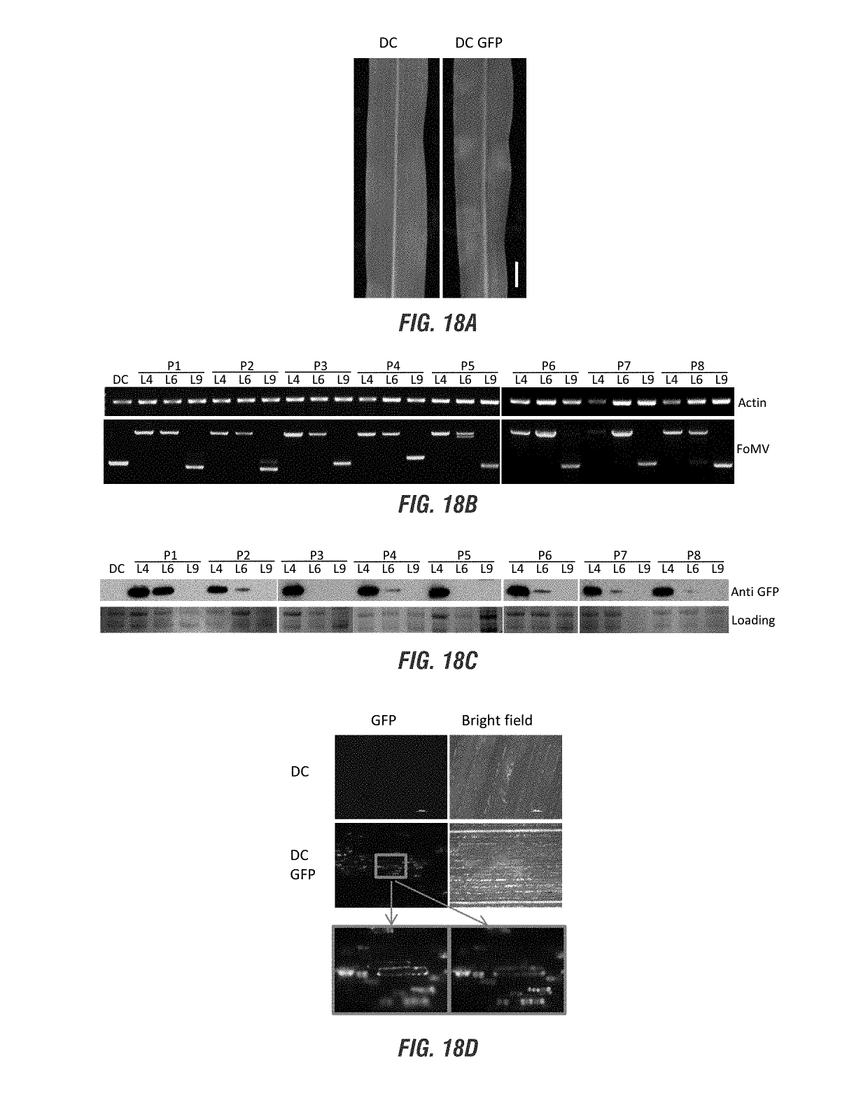

To further test the ability of FoMV to express foreign genes, we inserted the full length of GFP encoding gene into pFoMV-DC. The GFP gene was obtained by PCR using plasmid pSITE 2CA (Chakrabarty et al., 2007) as template and primer pairs GFP Bsu36I and GFP PspOMI. pGFP was generated by cloning the product into the pGEM T-easy vector. After sequencing verification, pGFP was digested by Bsu36I and PspOMI and ligated into similarly digested pFoMV-DC to generate the construct pFoMV-DC-GFP. GFP expression is only investigated using the pFoMV-DC vector since we see better stability in pFoMV-DC than pFoMV-DP in the BAR expression study. Sweet corn plants were inoculated with pFoMV-DC-GFP using biolistic bombardment. Infection by pFoMV-DC-GFP caused leaf mosaic symptom indistinguishable from pFoMV-DC infected plants (FIG. 18A).

The presence of GFP insertion and its stability was first tested by RT-PCR analysis using FoMV primers that flanked the cloning site. As shown in FIG. 18B, while a 475-bp PCR product was detected in the tissue infected by the empty vector, a 1192-bp PCR product was detected in the pFoMV-DC-GFP infected tissue, indicating the presence of intact GFP insertion. Of the 8 plants examined, no deletion was detected in the 4^{th} leaf samples, and 2 of the 6^{th} leaf samples showed minor deletion. However, complete deletion was detected in all the 9^{th} leaf samples.

Expression of GFP at the protein level was then analyzed by western blot using anti-GFP antibody. Consistent with the RT-PCR results, we discovered that GFP was detected in all the 4^{th} leaf samples and most of the 6^{th} leaf samples but not in any of the 9^{th} leaf samples where GFP insertion was totally lost. Also the signals from 4^{th} leaf samples were strongest in most of the plants, indicating that GFP expression is highest in 4^{th} leaves and decreasing in upper leaves.

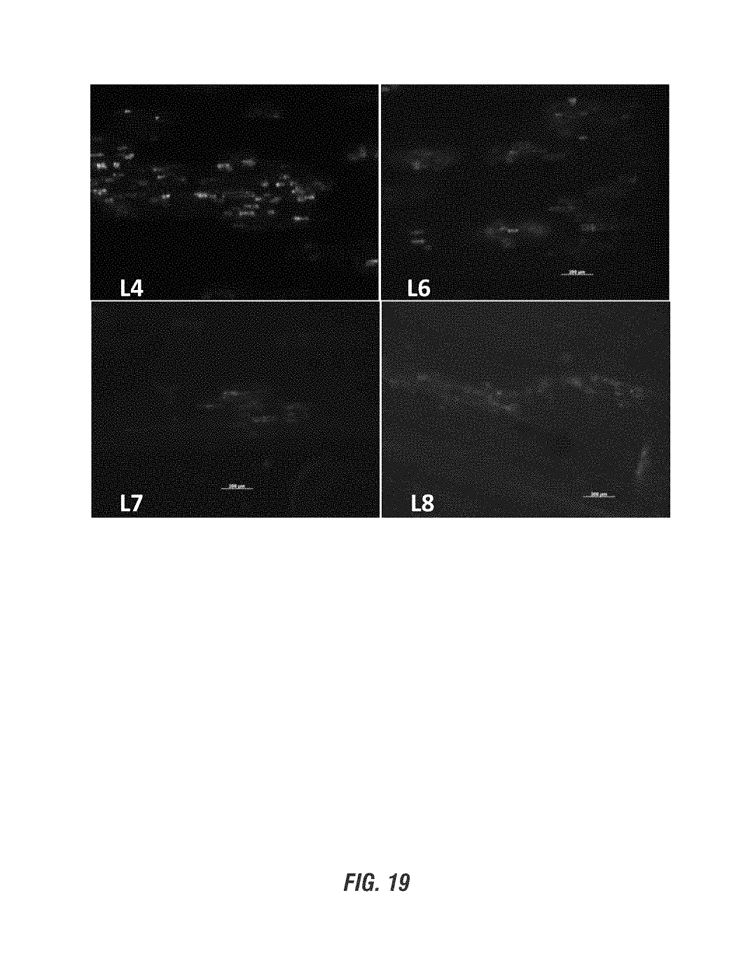

Further, when leaf areas with mosaic symptoms were examined under the microscope with UV light, green fluorescence was observed in pFoMV-DC-GFP-infected leaf tissues but not in leaf tissues infected by the empty vector. The green fluorescence observed was patchy, reflecting the pattern of FoMV movement (FIG. 18D). The signals were relatively strong in 4^{th} leaves and became weaker in upper leaves and finally undetectable in 9^{th} leaf (FIG. 19). In addition to epidermal cells where green fluorescence was detected in cytoplasm and nuclei, fluorescence was also detected in the underlying mesophyll cells.

### Example 9: Using FoMV Vector for Guide RNA Delivery

The results above demonstrated that the modified FoMV vectors pFoMV-DP and pFOMV-DC can be used for foreign protein expression. In addition, we were interested in whether these expression vectors can be used for guide RNA (gRNA) delivery, which would potentially facilitate targeted genome editing in plant species susceptible to FoMV. We first tested two gRNAs targeting the second exon of green foxtail Sish1 (Sevir.9G153200) and the first exon of green foxtail carbonic anhydrase 2 (CA2, Sevir.5G247900).

To generate constructs for gRNA delivery, oligonucleotides CA2OE1 & CA2OE2 were synthesized and annealed, ligated into pFoMV-DP or pFoMV-DC digested with Bsu36I and PspOMI to generate the construct pFoMV-DP-CA2 or pFoMV-DC-CA2. The construct pFoMV-DC-Sish1 was generated similarly using oligonucleotides Sish1OE1 & Sish1OE2. The gRNA delivery was also tried in the pFoMV-V vector where oligonucleotides CA2VIGS1 & CA2VIGS2, Sish1VIGS1 & Sish1VIGS2 were synthesized, annealed and ligated into pFoMV-V digested with XbaI and XhoI.

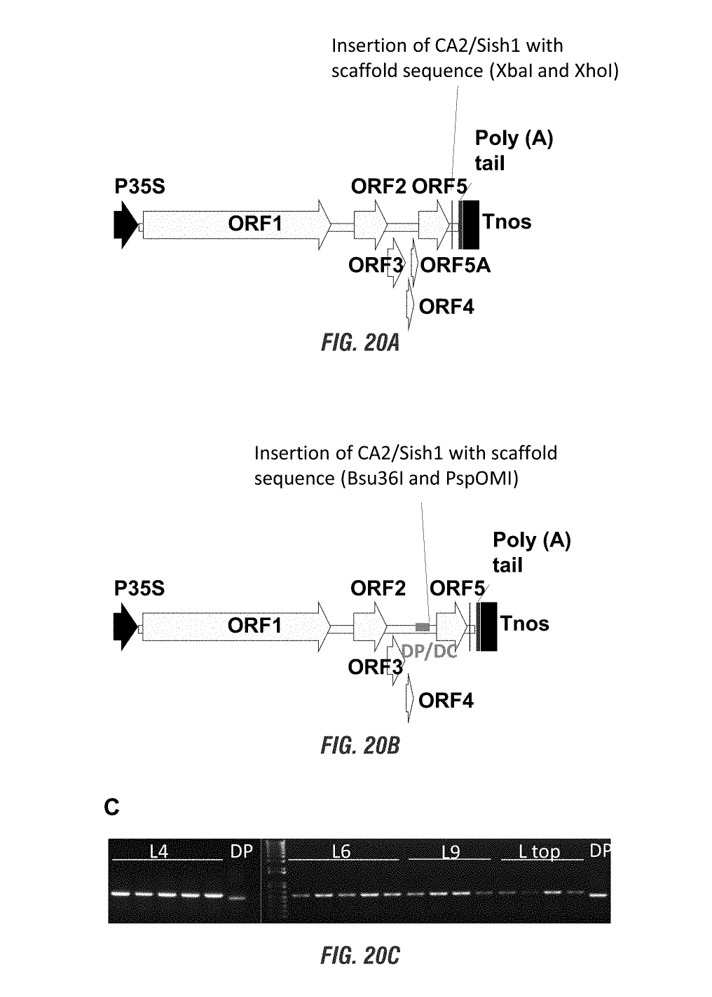

While Sish1 controls seed shattering, ectopic expression of carbonic anhydrase 2 causes male sterility caused by failure of anther dehiscence (Olsen, 2012; Villarreal et al., 2009). DNA fragments containing a 20-bp target sequence specific to Sish1 or CA2 followed by the 84-bp scaffold sequence for Cas9 binding was inserted in both FoMV-derived VIGS vector pFoMV-V (MCS I) and FoMV-derived expression vectors pFoMV-DP and pFoMV-DC (MCS II). To test the stability of the inserted fragment, RT-PCR analysis was performed on leaf tissues from sweet corn plants infected by pFoMV-DP-CA2. As shown in FIG. 20C, the insertion was detected in all infected plants and no deletion was detected in 4^{th}, 6^{th}, 9^{th}, or top leaf samples, indicating that the fragment is stable in maize leaves.

### Example 10: Development of an Agroinoculation Compatible FoMV Vector

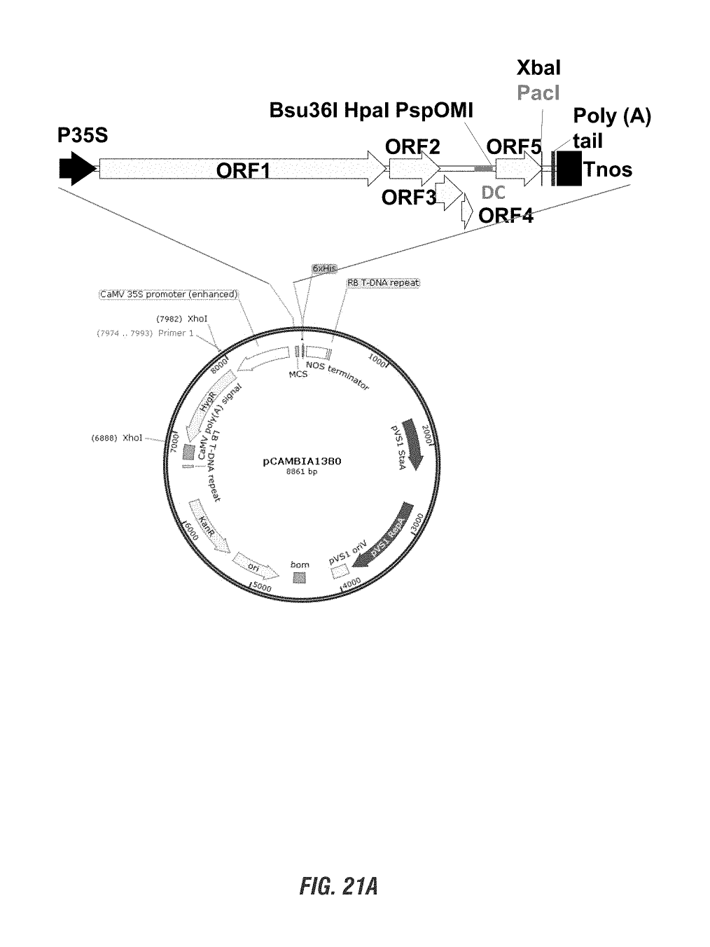

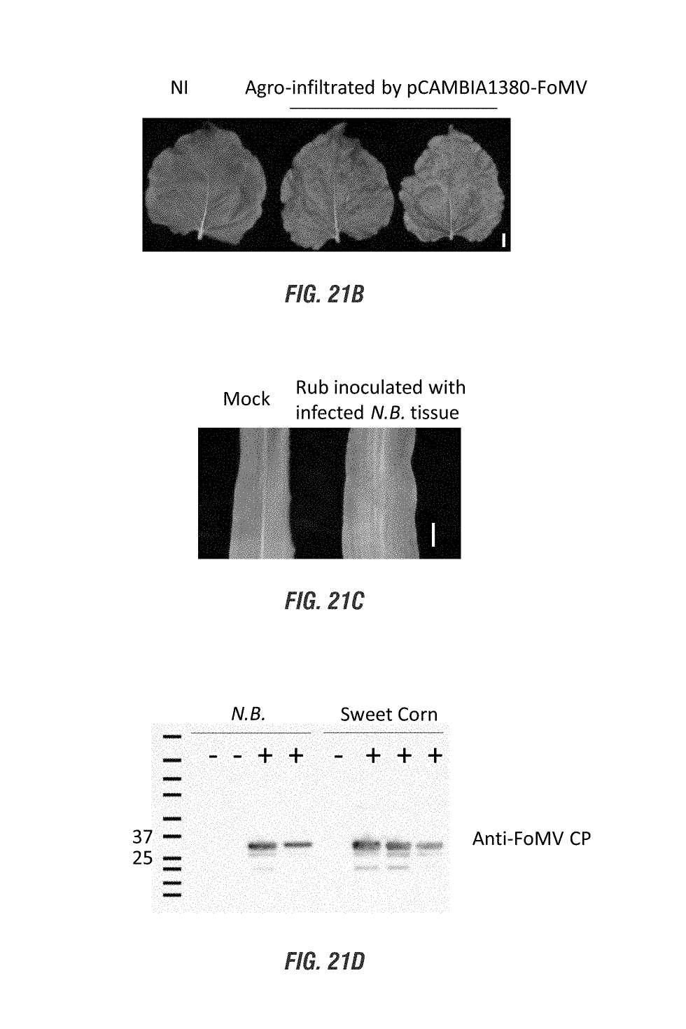

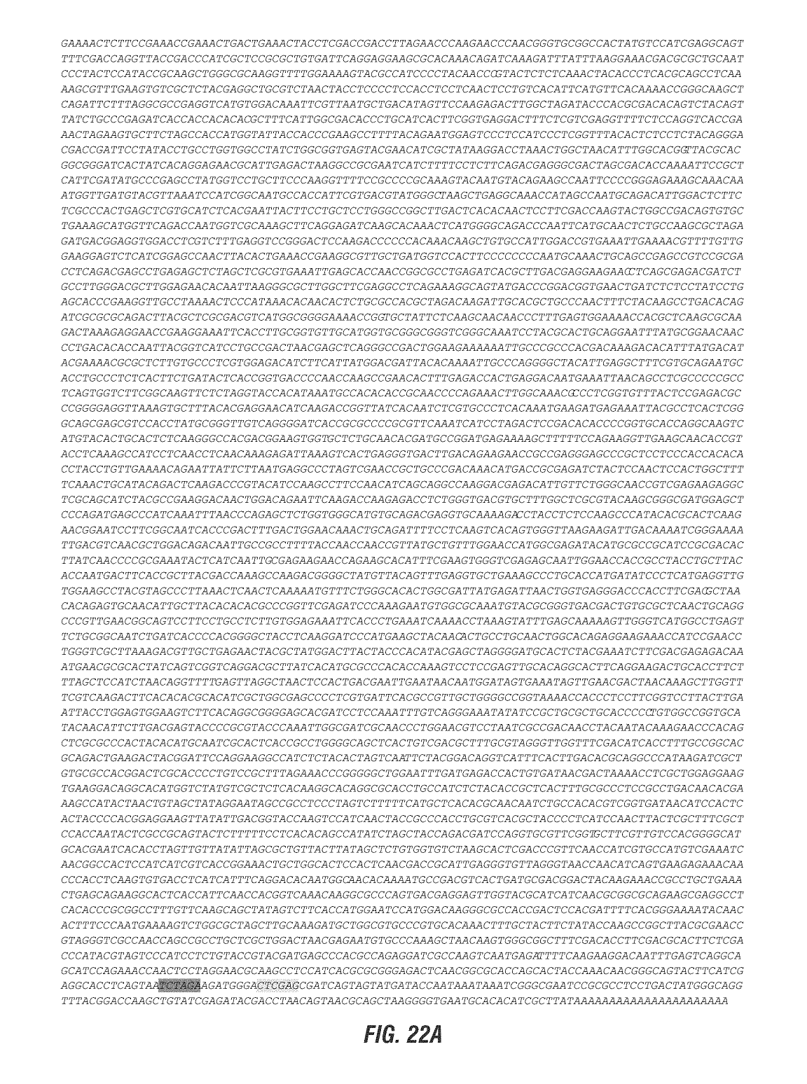

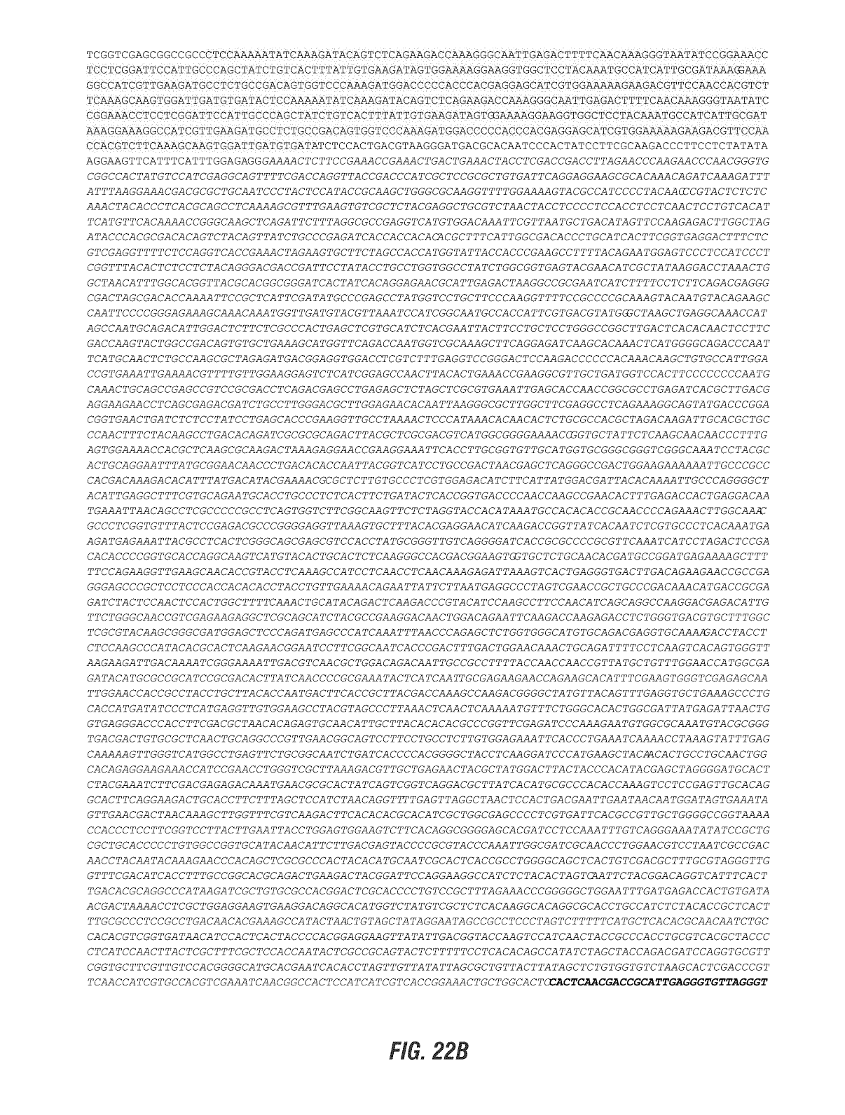

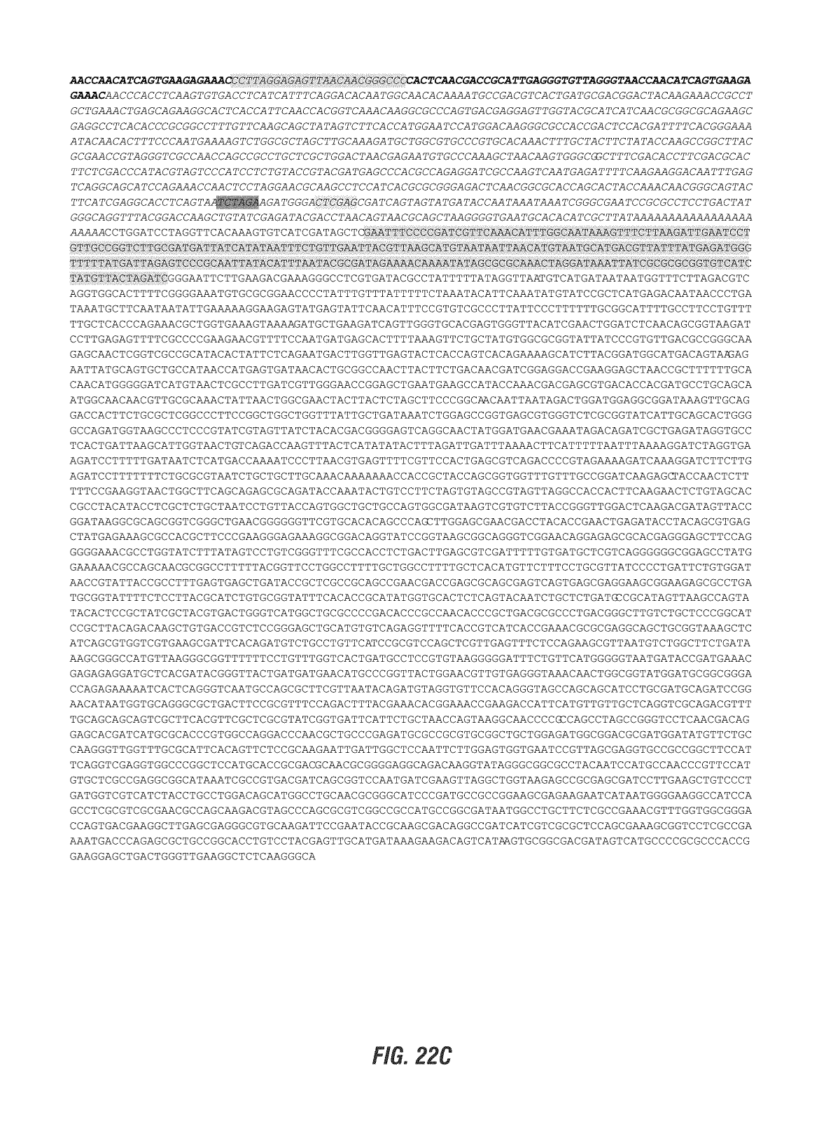

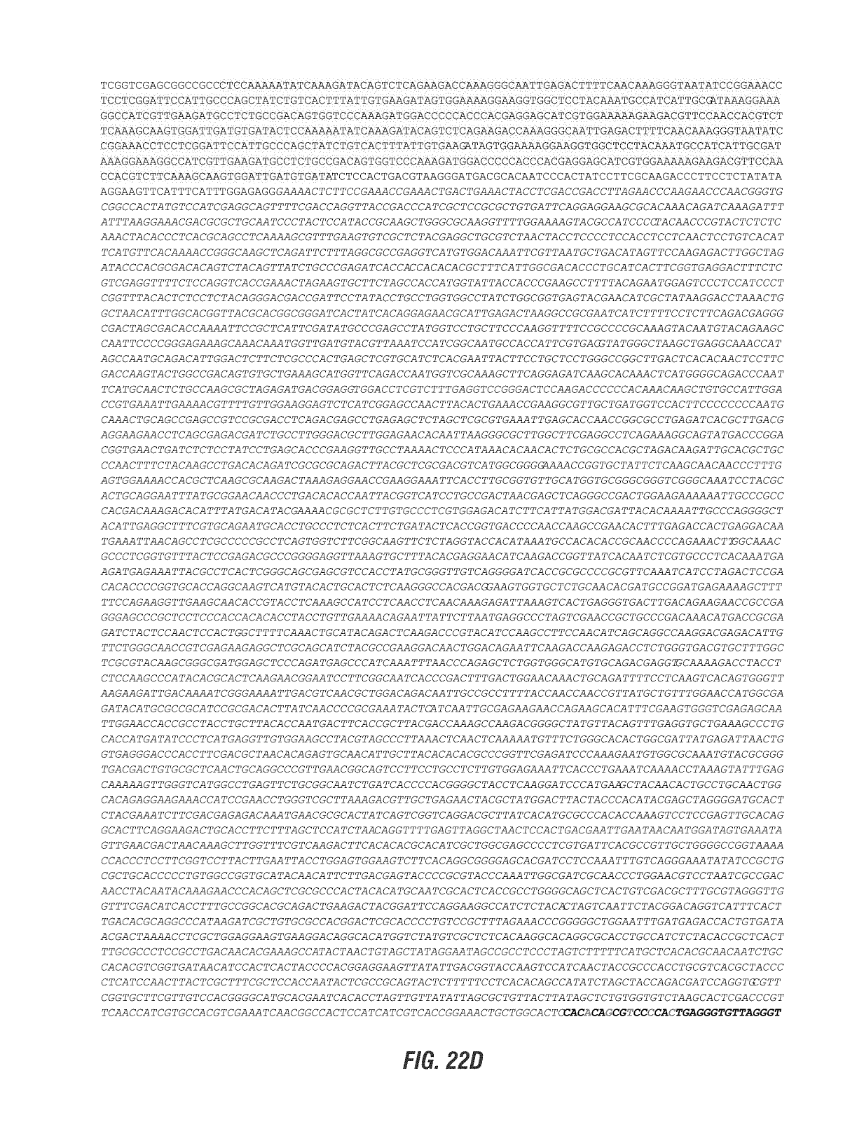

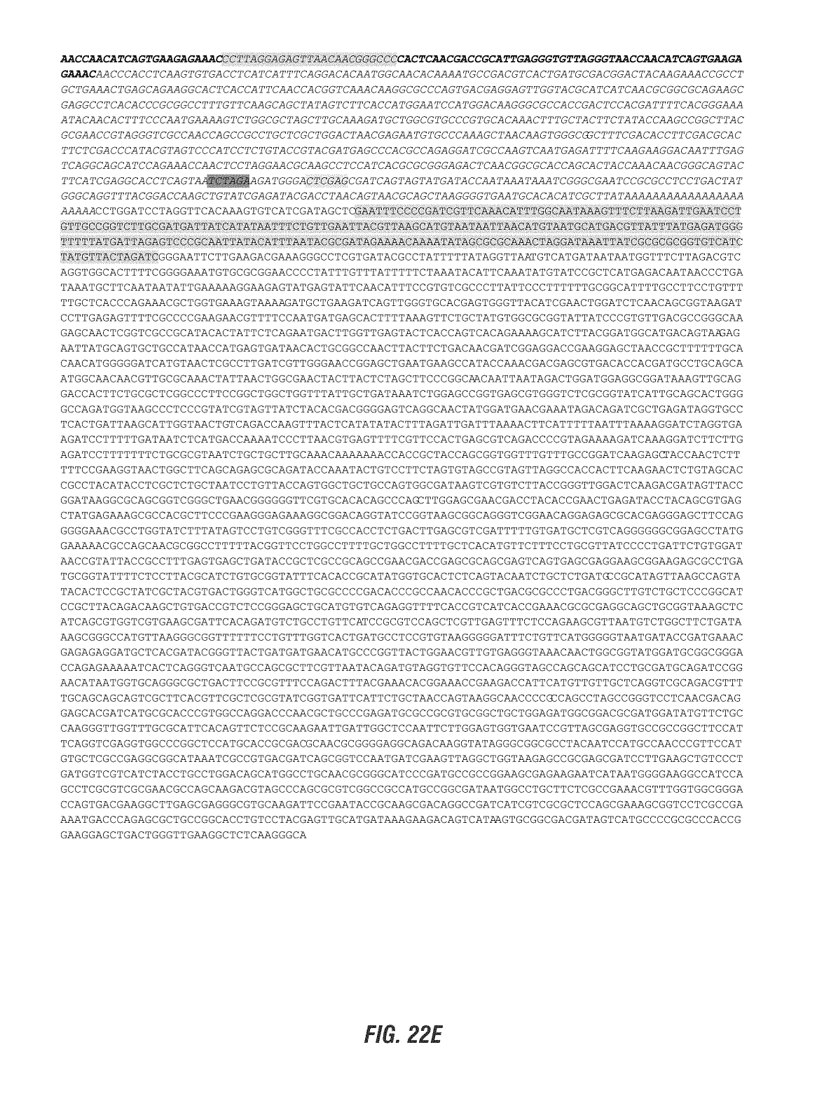

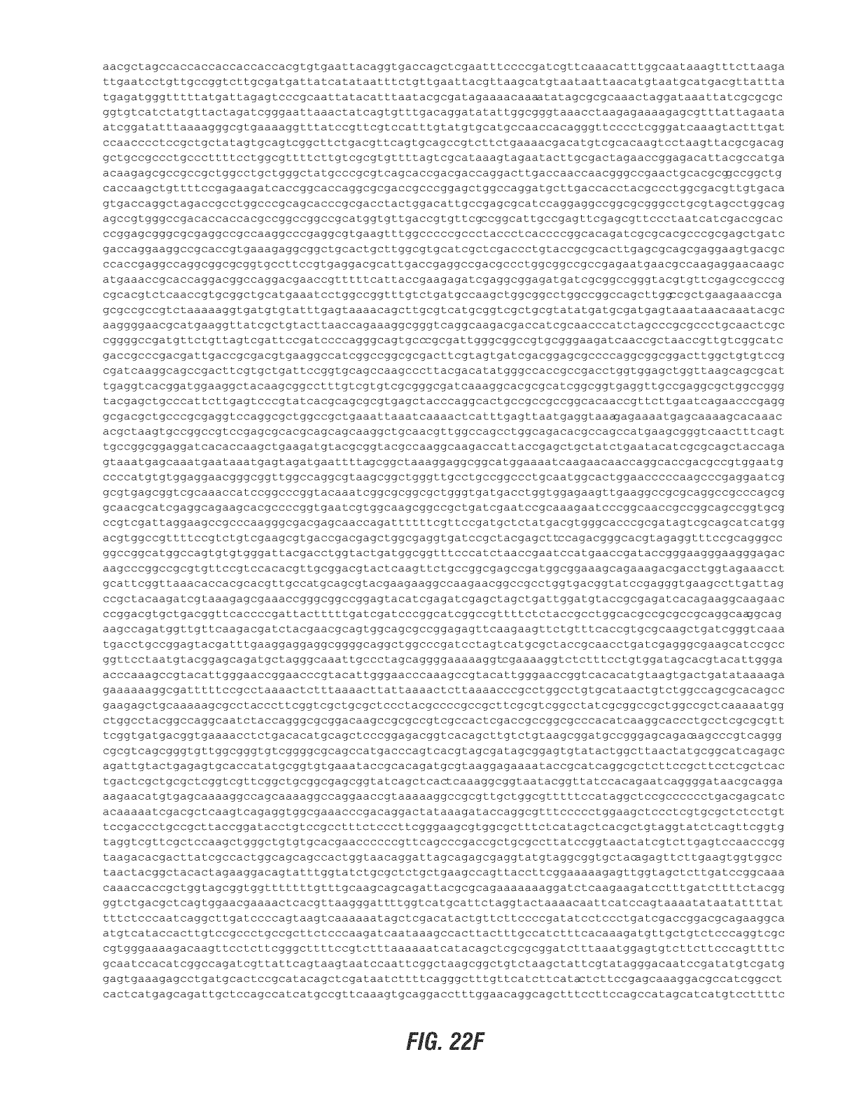

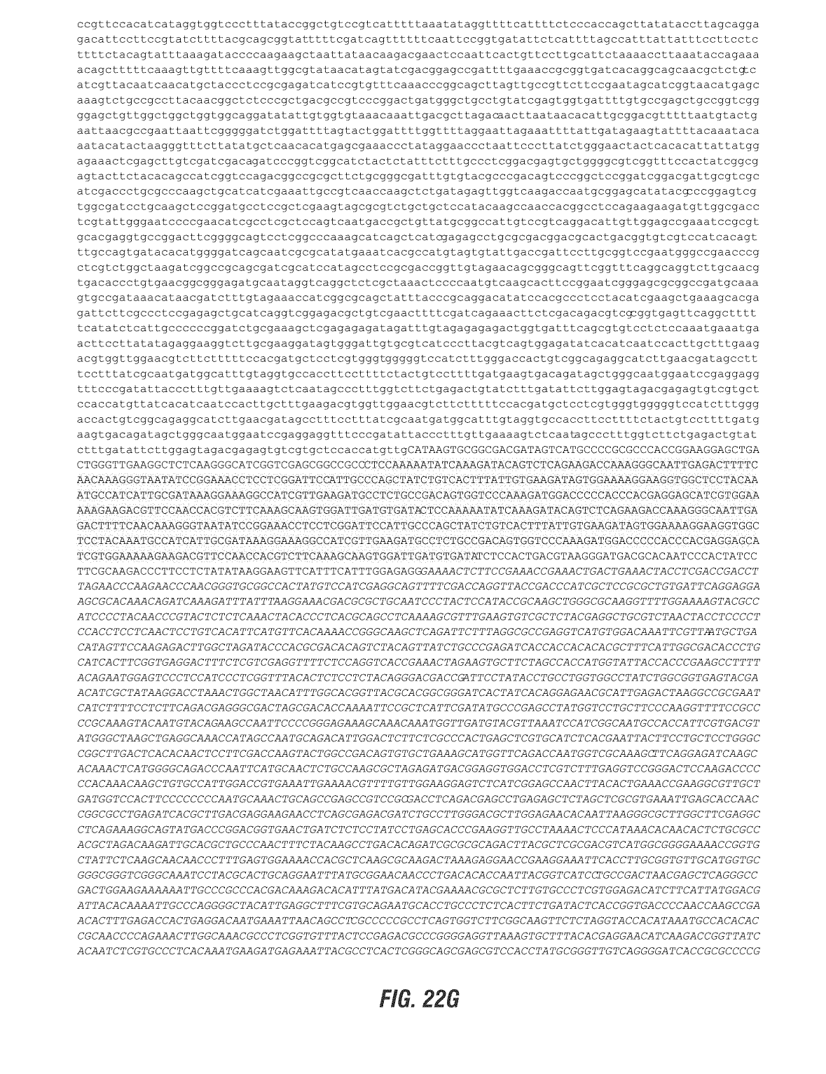

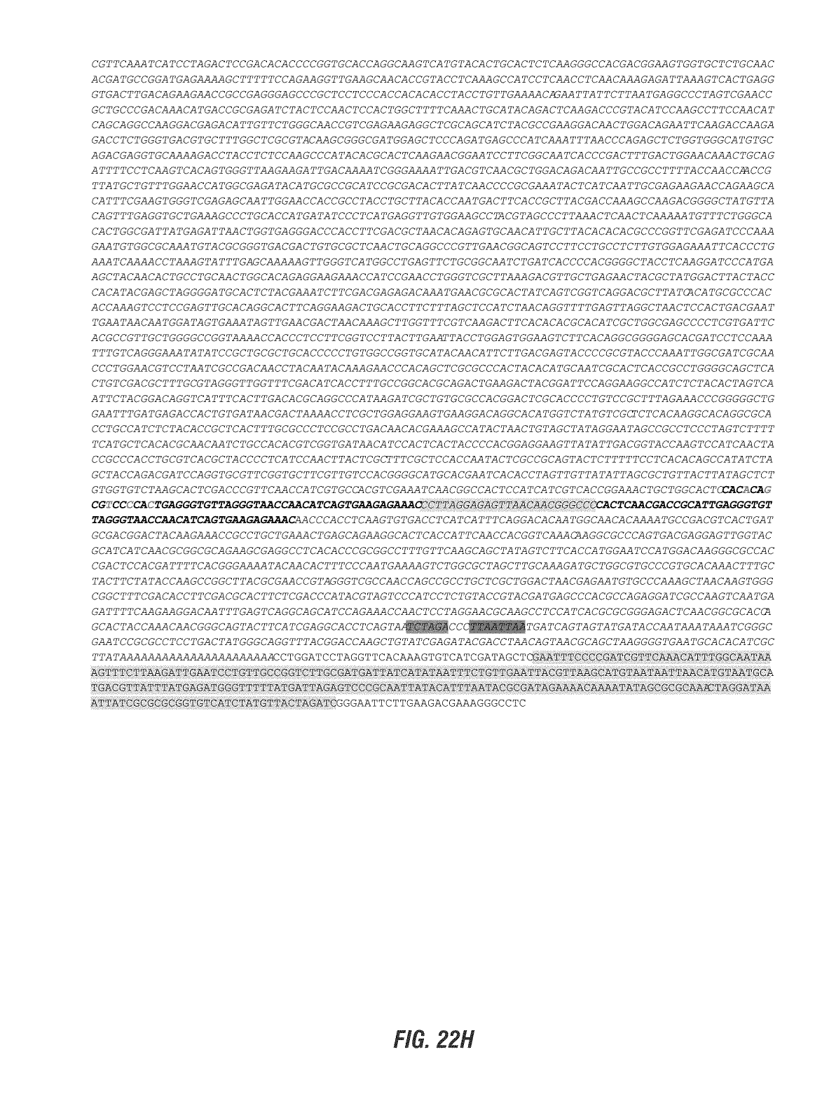

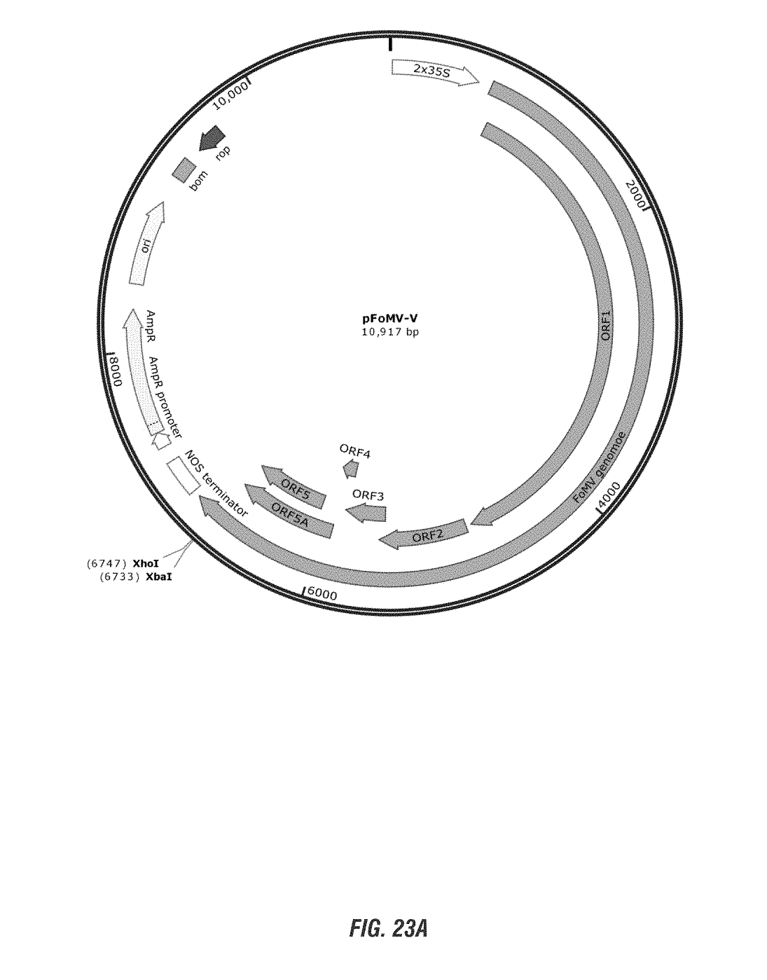

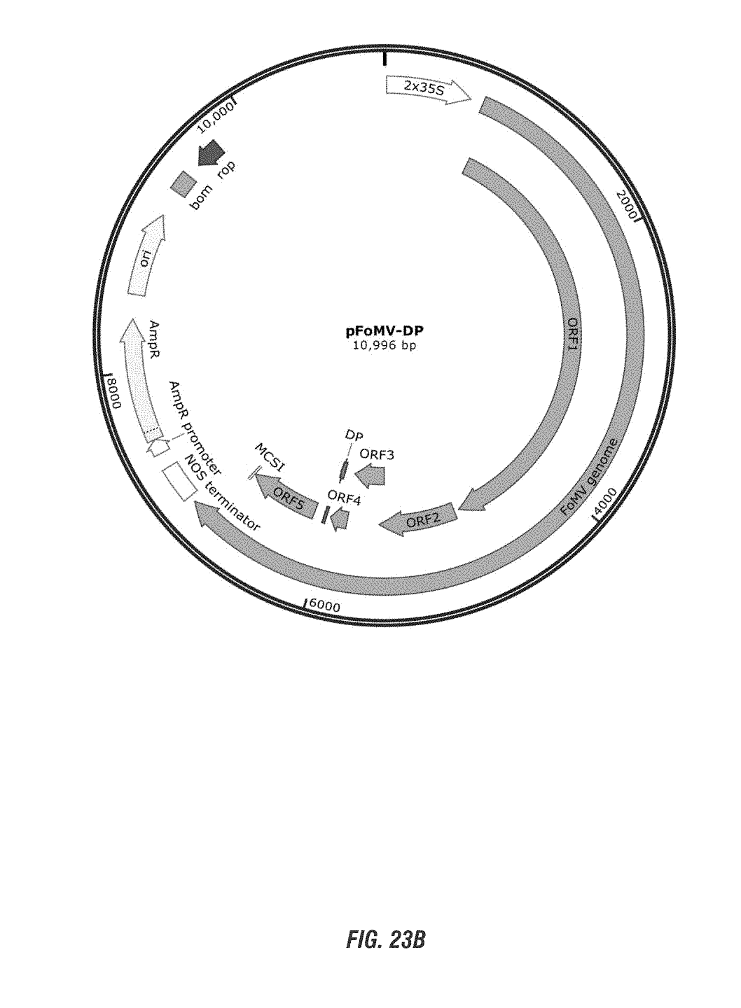

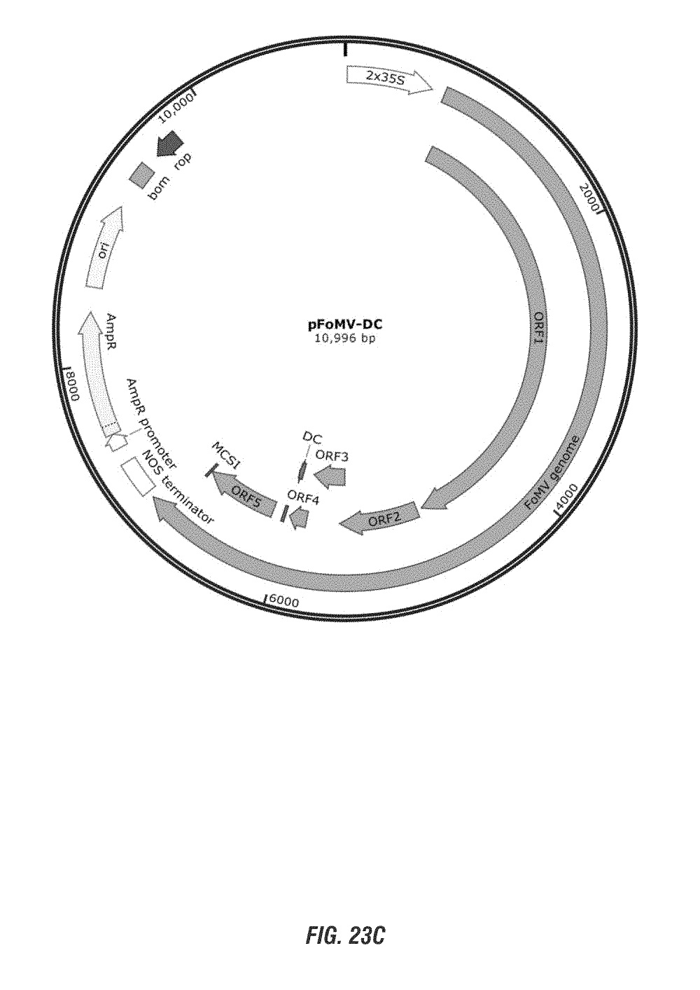

Both our FoMV-mediated VIGS and expression vectors are DNA-based, which are designed to be delivered directly into plant cells by biolistic inoculation. In order to make the vectors compatible with agroinoculation, which could simplify the inoculation procedure, the full length sequence of the modified FoMV genome together with the 35S promoter and Nos terminator was amplified and inserted into the binary vector pCAMBIA1380. Because the XhoI restriction endonuclease site is present in the backbone of pCAMBIA1380, it was substituted with PacI in the pFoMV-DC vector before the final assembly into the binary vector. The infectivity of the resultant pCAMBIA1380FoMV was then tested by infiltrating N. benthamiana plants with Agrobacterium-carrying pCAMBIA1380-FoMV. Mild leaf mosaic symptoms were observed on systemic N. benthamiana leaves at approximately 3 weeks after inoculation (FIG. 21B). When new sweet corn plants were rub-inoculated with sap of FoMV-infected N. benthamiana systemic leaves, leaf mosaic symptoms were observed in less than a week. FoMV infection in both the agroinfiltrated N. benthamiana plants and rub-inoculated new sweet corn plants were further confirmed by western blot using anti-FoMV-CP antibody (FIG. 21C). These results demonstrate that the pCAMBIA1380-FoMV is infectious through agroinoculation and the infected N. benthamiana leaves can be used as inoculum for passage onto corn plants.

TABLE I
Listing of primers and oligonucleotides

| Name | Sequence (5′-3′) | |
|---|---|---|
| FM-5end | GAAAACTCTTCCGAAACCGAAA | SEQ ID NO: 22 |
| | CTGACTGA | |
| FM-240F | GCCATCCCCTACAACCCGT | SEQ ID NO: 23 |
| FM-396R | GAATCTGAGCTTGCCCGGT | SEQ ID NO: 24 |
| FM-812F | AAGGCCGCGAATCATCTTT | SEQ ID NO: 25 |
| FM-1332F | GGACCGTGAAATTGAAAACGT | SEQ ID NO: 26 |
| FM-1888F | GCGGGCGGGTCGGGCA | SEQ ID NO: 27 |
| FM-2388F | CCCTCACAAATGAAGATGAGA | SEQ ID NO: 28 |
| FM-2388R | TTTCTCATCTTCATTTGTGAC | SEQ ID NO: 29 |
| FM-2923F | CGCCGAAGGACAACTGGACA | SEQ ID NO: 30 |
| FM-3450F | GAGGTACTGAAAGCCCTGCA | SEQ ID NO: 31 |
| FM-3998F | CGGTCAGGACGCTTATCACA | SEQ ID NO: 32 |
| FM-4539F | CGACATCACCTTTGCCGGCA | SEQ ID NO: 33 |
| FM-5074F | CCTCACACAGCCATATCTAGCT | SEQ ID NO: 34 |
| FM-5501F | ATCATCAACGCGGCGCAGA | SEQ ID NO: 35 |
| FM-6023F | CAGTGATCAGTAGTATGATACC | SEQ ID NO: 36 |
| | A | |
| FM-3end | TTTTTTTTTTTTTTTTTTTTTT | SEQ ID NO: 37 |
| | ATAAGCGATGTGTGCATTCA | |
| NosRev | AGACCGGCAACAGGATTCA | SEQ ID NO: 38 |
| FM-XbaRev | CTCGAGTCCCATCTTCTAGATT | SEQ ID NO: 39 |
| | ACTGAGGTGCCTCGATGAAGT | |
| FM-XhoFor | TAGAAGATGGGACTCGAGTGAT | SEQ ID NO: 40 |
| | CAGTAGTATGATACCAATAA | |
| FM-5840F | TCTGTACCGTACGATGAGCCC | SEQ ID NO: 41 |
| FM-6138R | GCTGCGTTACTGTTAGGTCG | SEQ ID NO: 42 |
| PDSVXb | TGCTCTAGAAGATGGGACGGGA | SEQ ID NO: 43 |
| | ACTTCTCCTGAT | |
| PDSVXh | CCGCTCGAGGCTAGCCAAGTTA | SEQ ID NO: 44 |
| | TTTCCTGATGA | |
| Les22VXb | GCTCTAGATCTGATCCCTTTTG | SEQ ID NO: 45 |
| | CGACCT | |
| Les22VXh | CCGCTCGAGACACATCCTGACT | SEQ ID NO: 46 |
| | TGCCTCT | |
| IjVXb | GCTCTAGACCAGGTTGTAGAAT | SEQ ID NO: 47 |
| | GCTCGC | |
| IjVXh | CCGCTCGAGGTGTACTTTGTGT | SEQ ID NO: 48 |
| | GAGGCGG | |
| Bm3VXb | GCTCTAGACTTGATGAACTCGA | SEQ ID NO: 49 |
| | TGGCCC | |
| Bm3VXh | CCGCTCGAGCACGCTGCTCAAG | SEQ ID NO: 50 |
| | AACTGTT | |
| ZmActrtS | GGTTTCGCTGGTGATGATGC | SEQ ID NO: 51 |
| ZmActrtA | CAATGCCATGCTCAATCGGG | SEQ ID NO: 52 |
| ZmActS | CCTGAAGATCACCCTGTGCT | SEQ ID NO: 53 |
| ZmActR | GCAGTCTCCAGCTCCTGTTC | SEQ ID NO: 54 |
| PDSrt3 | CAGCATTGAACGGTTTGGGTCA | SEQ ID NO: 55 |
| PDSrt4 | TGGAGAAGTTGGTGGGAGTTCC | SEQ ID NO: 56 |
| Les22rtS | GCTCCGTTTACCTTGGCATCT | SEQ ID NO: 57 |
| Les22rtA | CCCCATTGTCCGCTTGGTATT | SEQ ID NO: 58 |
| IjrtS | AACGAGACGGCAAAGGACAA | SEQ ID NO: 59 |
| IjrtA | GCTCTCAGTCTGCTTCCTCG | SEQ ID NO: 60 |
| Bm3rtS | GAACCAGGACAAGGTCCTCA | SEQ ID NO: 61 |
| Bm3rtA | AGTCCAGCAGCTTCTTGGTG | SEQ ID NO: 62 |
| 5AmuS1 | CGCCGACAACCTACAATACA | SEQ ID NO: 63 |
| 5AmuA1 | GATTTCGACGTGGCACGATG | SEQ ID NO: 64 |
| 5AmuS2 | CATCGTGCCACGTCGAAATC | SEQ ID NO: 65 |
| 5AmuA2 | CGTGAAAATCGTGGAGTCG | SEQ ID NO: 66 |
| 201DPA1 | GTTGTTAACTCTCCTAAGGACA | SEQ ID NO: 67 |
| | CGTGGAGTGCCAGCAGTTTC | |
| 201DPS1 | AGGAGAGTTAACAACGGGCCCC | SEQ ID NO: 68 |
| | ACTCAACGACCGCATTG | |
| DPS | TCAACGACCGCATTGAGGGTGT | SEQ ID NO: 69 |
| | TAGGGTAACCAACATCAGTGAA | |
| | GAGAAACCCTTAGGCACTTT | |
| DPA | AAAGTGCCTAAGGGTTTCTCTT | SEQ ID NO: 70 |
| | CACTGATGTTGGTTACCCTAAC | |
| | ACCCTCAATGCGGTCGTTGA | |
| DCS | ACAGCGTCCCCACTGAGGGTGT | SEQ ID NO: 71 |
| | TAGGGTAACCAACATCAGTGAA | |
| | GAGAAACCCTTAGGCACTTT | |
| DCA | AAAGTGCCTAAGGGTTTCTCTT | SEQ ID NO: 72 |
| | CACTGATGTTGGTTACCCTAAC | |
| | ACCCTCAGTGGGGACGCTGT | |
| GFP Bsu36I | CCTTAGGATGGTGAGCAAGGGA | SEQ ID NO: 73 |
| | GAGGA | |
| GFP PspOMI | GGGCCCTCACTTGTACAGCTCG | SEQ ID NO: 74 |
| | TCCATGC | |
| BAR Bsu36I | CCTTAGGATGAGCCCAGAACGA | SEQ ID NO: 75 |
| | CG | |
| BAR HpaI | GTTAACTCAGATCTCGGTGACG | SEQ ID NO: 76 |
| | GGCA | |
| Sish1OE1 | TTAGGATGTTCAGCATGCTATT | SEQ ID NO: 77 |
| | ACTGTTTTAGAGCTAGAAATAG | |
| | CAAGTTAAAATAAGGCTAGTCC | |
| | GTTATCAACTTGAAAAAGTGGC | |
| | ACCGAGTCGGTGCG | |
| Sish1OE2 | GGCCCGCACCGACTCGGTGCCA | SEQ ID NO: 78 |
| | CTTTTTCAAGTTGATAACGGAC | |
| | TAGCCTTATTTTAACTTGCTAT | |
| | TTCTAGCTCTAAAACAGTAATA | |
| | GCATGCTGAACATCC | |
| Sish1VIGS1 | CTAGAATGTTCAGCATGCTATT | SEQ ID NO: 79 |
| | ACTGTTTTAGAGCTAGAAATAG | |
| | CAAGTTAAAATAAGGCTAGTCC | |
| | GTTATCAACTTGAAAAAGTGGC | |
| | ACCGAGTCGGTGCC | |
| Sish1VIGS2 | TCGAGGCACCGACTCGGTGCCA | SEQ ID NO: 80 |
| | CTTTTTCAAGTTGATAACGGAC | |
| | TAGCCTTATTTTAACTTGCTAT | |
| | TTCTAGCTCTAAAACAGTAATA | |
| | GCATGCTGAACATT | |
| CA2OE1 | TTAGGTCGACGGTCGTGCAGCT | SEQ ID NO: 81 |
| | GGAGTTTTAGAGCTAGAAATAG | |
| | CAAGTTAAAATAAGGCTAGTCC | |
| | GTTATCAACTTGAAAAAGTGGC | |
| | ACCGAGTCGGTGCG | |
| CA2OE2 | GGCCCGCACCGACTCGGTGCCA | SEQ ID NO: 82 |
| | CTTTTTCAAGTTGATAACGGAC | |
| | TAGCCTTATTTTAACTTGCTAT | |
| | TTCTAGCTCTAAAACTCCAGCT | |
| | GCACGACCGTCGACC | |
| CA2VIGS1 | CTAGATCGACGGTCGTGCAGCT | SEQ ID NO: 83 |
| | GGAGTTTTAGAGCTAGAAATAG | |
| | CAAGTTAAAATAAGGCTAGTCC | |
| | GTTATCAACTTGAAAAAGTGGC | |
| | ACCGAGTCGGTGCC | |
| CA2VIGS2 | TCGAGGCACCGACTCGGTGCCA | SEQ ID NO: 84 |
| | CTTTTTCAAGTTGATAACGGAC | |
| | TAGCCTTATTTTAACTTGCTAT | |
| | TTCTAGCTCTAAAACTCCAGCT | |
| | GCACGACCGTCGAT | |
| FM-PacIFor | GCTCTAGACCCTTAATTAATGA | SEQ ID NO: 85 |
| | TCAGTAGTATGATACCAATAA | |
| 1380F | AACGCTAGCCACCACCAC | SEQ ID NO: 86 |
| 1380R | CAACATGGTGGAGCACGA | SEQ ID NO: 87 |
| DCPacI1380F | GAGAGTGTCGTGCTCCACCATG | SEQ ID NO: 88 |
| | TTGCATAAGTGCGGCGACGATA | |
| | G | |
| DCPacI1380R | CGTGGTGGTGGTGGTGGTGGCT | SEQ ID NO: 89 |
| | AGCGTTGAGGCCCTTTCGTCTT | |
| | CAAG | |

What is claimed is:

1. A nucleic acid construct for virus-induced gene silencing in plants comprising: a nucleic acid sequence encoding an infectious Foxtail mosaic virus (FoMV) with a functional movement encoding sequence operably linked to one or more regulatory elements functional in a plant cell.

2. The nucleic acid construct of claim 1, wherein said FoMV encoding nucleic acid sequence is a full-length FoMV sequence.

3. The nucleic acid construct of claim 1 wherein said FoMV sequence includes a multiple cloning site for insertion of a silencing sequence.

4. The nucleic acid construct of claim 1 further comprising a silencing sequence.

5. The nucleic acid construct of claim 1, wherein said plant is a monocot.

6. The nucleic acid construct of claim 1, wherein the regulatory element is a promoter.

7. The nucleic acid construct of claim 1 wherein said regulatory element is a terminator sequence.

8. The nucleic acid construct of claim 1, wherein the nucleic acid sequence encoding Foxtail mosaic virus (FoMV) is a full-length genomic sequence for Foxtail mosaic virus (FoMV).

9. The nucleic acid construct of claim 1 wherein said FoMV sequence is
(a) SEQ ID NO: 1, or 2 or a fragment thereof than encodes an infectious FoMV virus;
(b) the full length complement of any sequence in (a);
(c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.
(d) a polynucleotide that is at least 70% identical to the polynucleotide of (a) or (b) and which encodes an infectious FoMV;
(e) a polynucleotide that is at least 80% identical to the polynucleotide of (a) or (b) and which encodes an infectious FoMV;
(f) a polynucleotide that is at least 90% identical to the polynucleotide of (a) or (b) and which encodes an infectious FoMV; and
(g) a polynucleotide that is at least 95% identical to the polynucleotide of (a) or (b) and which encodes an infectious FoMV.

10. The nucleic acid construct of claim 2 wherein the silencing sequence is inserted in a multiple cloning site.

11. A vector comprising the nucleic acid construct of claim 1.

12. The vector of claim 11 wherein said vector is a plasmid vector.

13. A plant cell, tissue or organ comprising the nucleic acid construct of claim 1.

14. A nucleic acid construct for expression of a foreign nucleotide sequence in plants comprising:
a nucleic acid sequence encoding an infectious Foxtail mosaic virus (FoMV) with a functional movement encoding sequence operably linked to one or more regulatory elements functional in a plant cell.

15. The nucleic acid construct of claim 14, wherein said FoMV encoding nucleic acid sequence is a full-length FoMV sequence.

16. The nucleic acid construct of claim 14 wherein said FoMV sequence includes a multiple cloning site for insertion of a foreign nucleotide sequence.

17. The nucleic acid construct of claim 14 further comprising a foreign nucleotide sequence.

18. The nucleic acid construct of claim 17 wherein said foreign nucleotide sequence is a guide polynucleotide.

19. The nucleic acid construct of claim 18 wherein said guide polynucleotide is a guide RNA.

20. The nucleic acid construct of claim 14, wherein said plant is a monocot.

21. The nucleic acid construct of claim 14, wherein the regulatory element is a promoter.

22. The nucleic acid construct of claim 14 wherein said regulatory element is a terminator sequence.

23. The nucleic acid construct of claim 14, wherein the nucleic acid sequence encoding Foxtail mosaic virus (FoMV) is a full-length genomic sequence for Foxtail mosaic virus (FoMV).

24. The nucleic acid construct of claim 14 wherein said FoMV sequence is
(a) SEQ ID NO: 13, or 16 or a fragment thereof than encodes an infectious FoMV virus;
(b) the full length complement of any sequence in (a);
(c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.
(d) a polynucleotide that is at least 70% identical to the polynucleotide of (a) or (b) and which encodes an infectious FoMV;
(e) a polynucleotide that is at least 80% identical to the polynucleotide of (a) or (b) and which encodes an infectious FoMV;
(f) a polynucleotide that is at least 90% identical to the polynucleotide of (a) or (b) and which encodes an infectious FoMV; and
(g) a polynucleotide that is at least 95% identical to the polynucleotide of (a) or (b) and which encodes an infectious FoMV.

25. The nucleic acid construct of claim 15 wherein the foreign nucleotide sequence is inserted in a multiple cloning site.

26. A vector comprising the nucleic acid construct of claim 14.

27. The vector of claim 26 wherein said vector is a plasmid vector.

28. A plant cell, tissue or organ comprising the nucleic acid construct of claim 13.

29. A method of silencing a gene of interest in a plant cell comprising:
introducing to said plant cell a nucleic acid construct comprising a nucleic acid sequence encoding an infectious Foxtail mosaic virus (FoMV) with a functional movement encoding sequence operably linked to one or more regulatory elements functional in a plant cell, said Foxtail mosaic virus sequence having a silencing sequence inserted therein.

30. The method of claim 29, wherein the plant is a monocot.

31. The method of claim 29, wherein the gene to be silenced is inserted into the vector at a multiple cloning site following the nucleotide sequence for FoMV.

32. The method of claim 29, wherein the nucleotide sequence for FoMV is a full-length cDNA sequence for FoMV.

33. The method of claim 29, wherein the gene to be silenced is an endogenous gene of a plant.

34. The method of claim 29, wherein the introducing is by biolistics.

35. The method of claim 29, wherein the introducing is by agroinoculation.

36. A method of constructing a nucleic acid construct for gene silencing in a plant comprising:
inserting a multiple cloning site into a an infectious Foxtail mosaic virus (FoMV) sequence, wherein the cloning site is configured to receive a silencing nucleic acid sequence; and
operably linking a regulatory sequence functional in a plant cell to said FoMV sequence.

37. The method of claim 36, wherein the regulatory element is a promoter.

38. The method of claim 36 wherein said regulatory element is a terminator sequence.

39. The method of claim 36, wherein the nucleic acid sequence encoding Foxtail mosaic virus is a full-length genomic sequence for Foxtail mosaic virus.

40. The method of claim 36, wherein the nucleotide capable of silencing an endogenous gene is a gene to be silenced in reverse orientation.

41. A method of expressing a foreign gene of interest in a plant cell comprising:
introducing to said plant cell a nucleic acid construct comprising a nucleic acid sequence encoding an infectious Foxtail mosaic virus (FoMV) with a functional movement encoding sequence operably linked to one or more regulatory elements functional in a plant cell, said Foxtail mosaic virus sequence having foreign nucleotide sequence inserted therein.

42. The method of claim 41, wherein the plant is a monocot.

43. The method of claim 41, wherein the gene to be expressed is inserted into the vector at a multiple cloning site following the nucleotide sequence for FoMV.

44. The method of claim 41, wherein the nucleotide sequence for FoMV is a full-length cDNA sequence for FoMV.

45. The method of claim 41, wherein the introducing is by biolistics.

46. The method of claim 41, wherein the introducing is by agroinoculation.

47. A method of delivering a guide polynucleotide into a plant cell comprising:
introducing to said plant cell a nucleic acid construct comprising a nucleic acid sequence encoding an infectious Foxtail mosaic virus (FoMV) with a functional movement encoding sequence operably linked to one or more regulatory elements functional in a plant cell, said Foxtail mosaic virus sequence having a guide polynucleotide sequence inserted therein.

48. The method of claim 47, wherein the guide polynucleotide in a guide RNA.

49. The method of claim 47, wherein the plant is a monocot.

50. The method of claim 47, wherein the guide polynucleotide is inserted into the vector at a multiple cloning site following the nucleotide sequence for FoMV.

51. The method of claim 47, wherein the nucleotide sequence for FoMV is a full-length cDNA sequence for FoMV.

52. The method of claim 47, wherein the introducing is by biolistics.

53. The method of claim 47, wherein the introducing is by agroinoculation.


SEQUENCE LISTING (89 sequences)

SEQ ID NO: 1 - DNA, 6206 bp, Artificial Sequence
  Synthetic construct
gaaaactcttccgaaaccgaaactgactgaaactacctcgaccgaccttagaacccaagaacccaacgggtgcggccactatgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtaatctagaagatgggactcgagcgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaa

SEQ ID NO: 2 - DNA, 10916 bp, Artificial Sequence
  Synthetic construct vector
tcggtcgagcggccgccctccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagggaaaactcttccgaaaccgaaactgactgaaactacctcgaccgaccttagaacccaagaacccaacgggtgcggccactatgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtaatctagaagatgggactcgagcgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaacctggatcctaggttcacaaagtgtcatcgatagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggca

SEQ ID NO: 3 - DNA, 598 bp, Artificial Sequence
  Synthetic construct
ctccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgata

SEQ ID NO: 4 - DNA, 253 bp, Artificial Sequence
  Synthetic construct
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 5 - DNA, 10 bp, Artificial Sequence
  Restriction site
tctagannnn

SEQ ID NO: 6 - DNA, 10 bp, Artificial Sequence
  Restriction site
ctcgagnnnn

SEQ ID NO: 7 - DNA, 4008 bp, Foxtail mosaic virus
atgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaa

SEQ ID NO: 8 - DNA, 711 bp, Foxtail mosaic virus
atggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctga

SEQ ID NO: 9 - DNA, 384 bp, Foxtail mosaic virus
atgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctag

SEQ ID NO: 10 - DNA, 159 bp, Foxtail mosaic virus
atgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattga

SEQ ID NO: 11 - DNA, 801 bp, Foxtail mosaic virus
atgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtga

SEQ ID NO: 12 - DNA, 657 bp, Foxtail mosaic virus
atggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtga

SEQ ID NO: 13 - DNA, 10996 bp, Artificial Sequence
  Synthetic construct vector
tcggtcgagcggccgccctccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagggaaaactcttccgaaaccgaaactgactgaaactacctcgaccgaccttagaacccaagaacccaacgggtgcggccactatgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccacgtcgaaatcaacggccactccatcatcgtcaccggaaactgctggcactccactcaacgaccgcattgagggtgttagggtaaccaacatcagtgaagagaaacccttaggagagttaacaacgggccccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtaatctagaagatgggactcgagcgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaacctggatcctaggttcacaaagtgtcatcgatagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggca

SEQ ID NO: 14 - DNA, 25 bp, Artificial Sequence
  Synthetic construct
ccttaggagagttaacaacgggccc

SEQ ID NO: 15 - DNA, 54 bp, Foxtail mosaic virus
cactcaacgaccgcattgagggtgttagggtaaccaacatcagtgaagagaaac

SEQ ID NO: 16 - DNA, 10996 bp, Artificial Sequence
  Synthetic construct vector
tcggtcgagcggccgccctccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagggaaaactcttccgaaaccgaaactgactgaaactacctcgaccgaccttagaacccaagaacccaacgggtgcggccactatgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccacgtcgaaatcaacggccactccatcatcgtcaccggaaactgctggcactccacacagcgtccccactgagggtgttagggtaaccaacatcagtgaagagaaacccttaggagagttaacaacgggccccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtaatctagaagatgggactcgagcgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaacctggatcctaggttcacaaagtgtcatcgatagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggca

SEQ ID NO: 17 - DNA, 54 bp, Artificial Sequence
  Synthetic construct
cacacagcgtccccactgagggtgttagggtaaccaacatcagtgaagagaaac

SEQ ID NO: 18 - DNA, 16164 bp, Artificial Sequence
  Synthetic construct vector
aacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgcataagtgcggcgacgatagtcatgccccgcgcccaccggaaggagctgactgggttgaaggctctcaagggcatcggtcgagcggccgccctccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagggaaaactcttccgaaaccgaaactgactgaaactacctcgaccgaccttagaacccaagaacccaacgggtgcggccactatgtccatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggctgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtactggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctcagaaaggcagtatgacccggacggtgaactgatctctcctatcctgagcacccgaaggttgcctaaaactcccataaacacaacactctgcgccacgctagacaagattgcacgctgcccaactttctacaagcctgacacagatcgcgcgcagacttacgctcgcgacgtcatggcggggaaaaccggtgctattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctacctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcaccgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccacgtcgaaatcaacggccactccatcatcgtcaccggaaactgctggcactccacacagcgtccccactgagggtgttagggtaaccaacatcagtgaagagaaacccttaggagagttaacaacgggccccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtaatctagacccttaattaatgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaacctggatcctaggttcacaaagtgtcatcgatagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcttgaagacgaaagggcctc

SEQ ID NO: 19 - DNA, 8777 bp, Artificial Sequence
  Synthetic construct vector
aacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttg

SEQ ID NO: 20 - DNA, 314 bp, Artificial Sequence
  Synthetic construct
atgcacgaatcacacctagttgttatattagcgctgttacttatagctctgtggtgtctaagcactcgacccgttcaaccatcgtgccacgtcgaaatcaacggccactccatcatcgtcaccggaaactgctggcactccactcaacgaccgcattgagggtgttagggtaaccaacatcagtgaagagaaacccttaggagagttaacaacgggccccactcaacgaccgcattgagggtgttagggtaaccaacatcagtgaagagaaacaacccacctcaagtgtgacctcatcatttcaggacacaatg

SEQ ID NO: 21 - DNA, 314 bp, Artificial Sequence
  Synthetic construct
atgcacgaatcacacctagttgttatattagcgctgttacttatagctctgtggtgtctaagcactcgacccgttcaaccatcgtgccacgtcgaaatcaacggccactccatcatcgtcaccggaaactgctggcactccacacagcgtccccactgagggtgttagggtaaccaacatcagtgaagagaaacccttaggagagttaacaacgggccccactcaacgaccgcattgagggtgttagggtaaccaacatcagtgaagagaaacaacccacctcaagtgtgacctcatcatttcaggacacaatg

SEQ ID NO: 22 - DNA, 30 bp, Foxtail mosaic virus
gaaaactcttccgaaaccgaaactgactga

SEQ ID NO: 23 - DNA, 19 bp, Foxtail mosaic virus
gccatcccctacaacccgt

SEQ ID NO: 24 - DNA, 19 bp, Foxtail mosaic virus
gaatctgagcttgcccggt

SEQ ID NO: 25 - DNA, 19 bp, Foxtail mosaic virus
aaggccgcgaatcatcttt

SEQ ID NO: 26 - DNA, 21 bp, Foxtail mosaic virus
ggaccgtgaaattgaaaacgt

SEQ ID NO: 27 - DNA, 16 bp, Foxtail mosaic virus
gcgggcgggtcgggca

SEQ ID NO: 28 - DNA, 21 bp, Foxtail mosaic virus
ccctcacaaatgaagatgaga

SEQ ID NO: 29 - DNA, 21 bp, Foxtail mosaic virus
tttctcatcttcatttgtgac

SEQ ID NO: 30 - DNA, 20 bp, Foxtail mosaic virus
cgccgaaggacaactggaca

SEQ ID NO: 31 - DNA, 20 bp, Foxtail mosaic virus
gaggtactgaaagccctgca

SEQ ID NO: 32 - DNA, 20 bp, Foxtail mosaic virus
cggtcaggacgcttatcaca

SEQ ID NO: 33 - DNA, 20 bp, Foxtail mosaic virus
cgacatcacctttgccggca

SEQ ID NO: 34 - DNA, 22 bp, Foxtail mosaic virus
cctcacacagccatatctagct

SEQ ID NO: 35 - DNA, 19 bp, Foxtail mosaic virus
atcatcaacgcggcgcaga

SEQ ID NO: 36 - DNA, 23 bp, Foxtail mosaic virus
cagtgatcagtagtatgatacca

SEQ ID NO: 37 - DNA, 42 bp, Foxtail mosaic virus
ttttttttttttttttttttttataagcgatgtgtgcattca

SEQ ID NO: 38 - DNA, 19 bp, Artificial Sequence
  Synthetic construct primer
agaccggcaacaggattca

SEQ ID NO: 39 - DNA, 43 bp, Artificial Sequence
  Synthetic construct primer
ctcgagtcccatcttctagattactgaggtgcctcgatgaagt

SEQ ID NO: 40 - DNA, 42 bp, Artificial Sequence
  Synthetic construct primer
tagaagatgggactcgagtgatcagtagtatgataccaataa

SEQ ID NO: 41 - DNA, 21 bp, Foxtail mosaic virus
tctgtaccgtacgatgagccc

SEQ ID NO: 42 - DNA, 20 bp, Foxtail mosaic virus
gctgcgttactgttaggtcg

SEQ ID NO: 43 - DNA, 34 bp, Artificial Sequence
  Synthetic construct primer
tgctctagaagatgggacgggaacttctcctgat

SEQ ID NO: 44 - DNA, 33 bp, Artificial Sequence
  Synthetic construct primer
ccgctcgaggctagccaagttatttcctgatga

SEQ ID NO: 45 - DNA, 28 bp, Artificial Sequence
  Synthetic construct primer
gctctagatctgatcccttttgcgacct

SEQ ID NO: 46 - DNA, 29 bp, Artificial Sequence
  Synthetic construct primer
ccgctcgagacacatcctgacttgcctct

SEQ ID NO: 47 - DNA, 28 bp, Artificial Sequence
  Synthetic construct primer
gctctagaccaggttgtagaatgctcgc

SEQ ID NO: 48 - DNA, 29 bp, Artificial Sequence
  Synthetic construct primer
ccgctcgaggtgtactttgtgtgaggcgg

SEQ ID NO: 49 - DNA, 28 bp, Artificial Sequence
  Synthetic construct primer
gctctagacttgatgaactcgatggccc

SEQ ID NO: 50 - DNA, 29 bp, Artificial Sequence
  Synthetic construct primer
ccgctcgagcacgctgctcaagaactgtt

SEQ ID NO: 51 - DNA, 20 bp, Zea mays
ggtttcgctggtgatgatgc

SEQ ID NO: 52 - DNA, 20 bp, Zea mays
caatgccatgctcaatcggg

SEQ ID NO: 53 - DNA, 20 bp, Zea mays
cctgaagatcaccctgtgct

SEQ ID NO: 54 - DNA, 20 bp, Zea mays
gcagtctccagctcctgttc

SEQ ID NO: 55 - DNA, 22 bp, Zea mays
cagcattgaacggtttgggtca

SEQ ID NO: 56 - DNA, 22 bp, Zea mays
tggagaagttggtgggagttcc

SEQ ID NO: 57 - DNA, 21 bp, Zea mays
gctccgtttaccttggcatct

SEQ ID NO: 58 - DNA, 21 bp, Zea mays
ccccattgtccgcttggtatt

SEQ ID NO: 59 - DNA, 20 bp, Zea mays
aacgagacggcaaaggacaa

SEQ ID NO: 60 - DNA, 20 bp, Zea mays
gctctcagtctgcttcctcg

SEQ ID NO: 61 - DNA, 20 bp, Zea mays
gaaccaggacaaggtcctca

SEQ ID NO: 62 - DNA, 20 bp, Zea mays
agtccagcagcttcttggtg

SEQ ID NO: 63 - DNA, 20 bp, Artificial Sequence
  Synthetic construct primer
cgccgacaacctacaataca

SEQ ID NO: 64 - DNA, 20 bp, Artificial Sequence
  Synthetic construct primer
gatttcgacgtggcacgatg

SEQ ID NO: 65 - DNA, 20 bp, Artificial Sequence
  Synthetic construct primer
catcgtgccacgtcgaaatc

SEQ ID NO: 66 - DNA, 19 bp, Artificial Sequence
  Synthetic construct primer
cgtgaaaatcgtggagtcg

SEQ ID NO: 67 - DNA, 42 bp, Artificial Sequence
  Synthetic construct primer
gttgttaactctcctaaggacacgtggagtgccagcagtttc

SEQ ID NO: 68 - DNA, 39 bp, Artificial Sequence
  Synthetic construct primer
aggagagttaacaacgggccccactcaacgaccgcattg

SEQ ID NO: 69 - DNA, 64 bp, Artificial Sequence
  Synthetic construct
tcaacgaccgcattgagggtgttagggtaaccaacatcagtgaagagaaacccttaggcacttt

SEQ ID NO: 70 - DNA, 64 bp, Artificial Sequence
  Synthetic construct
aaagtgcctaagggtttctcttcactgatgttggttaccctaacaccctcaatgcggtcgttga

SEQ ID NO: 71 - DNA, 64 bp, Artificial Sequence
  Synthetic construct
acagcgtccccactgagggtgttagggtaaccaacatcagtgaagagaaacccttaggcacttt

SEQ ID NO: 72 - DNA, 64 bp, Artificial Sequence
  Synthetic construct
aaagtgcctaagggtttctcttcactgatgttggttaccctaacaccctcagtggggacgctgt

SEQ ID NO: 73 - DNA, 27 bp, Artificial Sequence
  Synthetic construct primer
ccttaggatggtgagcaagggagagga

SEQ ID NO: 74 - DNA, 29 bp, Artificial Sequence
  Synthetic construct primer
gggccctcacttgtacagctcgtccatgc

SEQ ID NO: 75 - DNA, 24 bp, Artificial Sequence
  Synthetic construct primer
ccttaggatgagcccagaacgacg

SEQ ID NO: 76 - DNA, 26 bp, Artificial Sequence
  Synthetic construct primer
gttaactcagatctcggtgacgggca

SEQ ID NO: 77 - DNA, 102 bp, Artificial Sequence
  Synthetic construct
ttaggatgttcagcatgctattactgttttagagctagaaatagcaagttaaaataaggctagtccgttatcaacttgaaaaagtggcaccgagtcggtgcg

SEQ ID NO: 78 - DNA, 103 bp, Artificial Sequence
  Synthetic construct
ggcccgcaccgactcggtgccactttttcaagttgataacggactagccttattttaacttgctatttctagctctaaaacagtaatagcatgctgaacatcc

SEQ ID NO: 79 - DNA, 102 bp, Artificial Sequence
  Synthetic construct
ctagaatgttcagcatgctattactgttttagagctagaaatagcaagttaaaataaggctagtccgttatcaacttgaaaaagtggcaccgagtcggtgcc

SEQ ID NO: 80 - DNA, 102 bp, Artificial Sequence
  Synthetic construct
tcgaggcaccgactcggtgccactttttcaagttgataacggactagccttattttaacttgctatttctagctctaaaacagtaatagcatgctgaacatt

SEQ ID NO: 81 - DNA, 102 bp, Artificial Sequence
  Synthetic construct
ttaggtcgacggtcgtgcagctggagttttagagctagaaatagcaagttaaaataaggctagtccgttatcaacttgaaaaagtggcaccgagtcggtgcg

SEQ ID NO: 82 - DNA, 103 bp, Artificial Sequence
  Synthetic construct
ggcccgcaccgactcggtgccactttttcaagttgataacggactagccttattttaacttgctatttctagctctaaaactccagctgcacgaccgtcgacc

SEQ ID NO: 83 - DNA, 102 bp, Artificial Sequence
  Synthetic construct
ctagatcgacggtcgtgcagctggagttttagagctagaaatagcaagttaaaataaggctagtccgttatcaacttgaaaaagtggcaccgagtcggtgcc

SEQ ID NO: 84 - DNA, 102 bp, Artificial Sequence
  Synthetic construct
tcgaggcaccgactcggtgccactttttcaagttgataacggactagccttattttaacttgctatttctagctctaaaactccagctgcacgaccgtcgat

SEQ ID NO: 85 - DNA, 43 bp, Artificial Sequence
  Synthetic construct primer
gctctagacccttaattaatgatcagtagtatgataccaataa

SEQ ID NO: 86 - DNA, 18 bp, Artificial Sequence
  Synthetic construct primer
aacgctagccaccaccac

SEQ ID NO: 87 - DNA, 18 bp, Artificial Sequence
  Synthetic construct primer
caacatggtggagcacga

SEQ ID NO: 88 - DNA, 45 bp, Artificial Sequence
  Synthetic construct primer
gagagtgtcgtgctccaccatgttgcataagtgcggcgacgatag

SEQ ID NO: 89 - DNA, 48 bp, Artificial Sequence
  Synthetic construct primer
cgtggtggtggtggtggtggctagcgttgaggccctttcgtcttcaag